(19) United States
(12) Patent Application Publication
(10) Pub. No.: US 2026/0184804 A1
(43) Pub. Date: Jul. 2, 2026

(54) VHH ANTIBODIES AND USES THEREOF

(71) Applicant: KEY2BRAIN AB, Solna (SE)

(72) Inventors: Elisabet OHLIN SJÖSTRÖM, Stockholm (SE); Magnus BERGLUND, Vendelsö (SE); Erik NORDLING, Danderyd (SE); Mikael ÅSTRAND, Sollentuna (SE)

(21) Appl. No.: 19/131,736

(22) PCT Filed: Nov. 22, 2023
(86) PCT No.: PCT/SE2023/051179

§ 371 Date: May 21, 2025

(30) Foreign Application Priority Data
Nov. 23, 2022 (SE) 2251368-2

(51) Int. Cl. C07K 16/28; A61K 39/00; A61P 25/00

(52) U.S. Cl. CPC C07K 16/2881; A61P 25/00; A61K 2039/505; C07K 2317/22; C07K 2317/24; C07K 2317/34; C07K 2317/35; C07K 2317/52; C07K 2317/569; C07K 2317/622; C07K 2317/77; C07K 2317/92; C07K 2319/00

(57) ABSTRACT

The present invention relates to variable domain of heavy chain-only (VHH) antibody binding specifically to the transferrin receptor 1 (TfR1) and uses thereof to transport therapeutic agents or imaging agents into cells and over the blood brain barrier.

### TECHNICAL FIELD

The invention relates to variable domain of heavy chain-only (VHH) antibodies that are able to bind the transferrin receptor, and to the use of such VHH antibodies to transport molecules across the blood-brain barrier to relevant targets in the brain.

### BACKGROUND

Brain exposure of drugs to target diseases of the central nervous system (CNS) is inherently difficult since the blood-brain barrier (BBB) protects the brain from unwanted substances present in the peripheral circulation, including antibodies and other proteins that may have a therapeutic effect within the brain. There are also small molecules that due to their properties, such as lack of lipophilicity or substrates to efflux pumps, are excluded from the brain compartment. Normally, a compound with a size under 600 Daltons (Da) can pass the BBB if not excluded by other forces. Larger molecules, such as proteins, do not readily pass into the brain in any considerable amounts if not helped by specific active transport. For therapeutic antibodies and protein-based drugs less than 0.1% of systemically injected therapeutic antibodies is estimated to reach the brain compartment. Several strategies to overcome this tight barrier have been tested and evaluated.

The BBB is composed of brain endothelial cells (BECs) as a first obstacle to entering the brain. Other cells of the so-called neurovascular unit (NVU) are also of importance for the transport and interplay to target cells in the brain parenchyma. In the human brain, the vessels of the brain span a total of 20 m², representing a large surface area, which can present a circulating therapeutic an opportunity for brain exposure.

Therapeutic antibodies or other protein-based drugs have a great potential to treat pathologies of the CNS. However, the low availability of such therapeutic molecules in the brain compartment is a major problem. Recently, therapeutic monoclonal antibodies with targets within the brain, such as amyloid beta protofibrils, have reported clinical effect. Nonetheless, the exposure in the human brain compartment of these therapeutic molecules after each administration is not regarded to be at their optimum.

There is, thus, a need to increase exposure of therapeutic molecules to the brain in order to improve the safety, dosage and total costs of CNS therapeutics.

Receptor-mediated transcytosis, a natural mechanism using endogenous receptors expressed at the luminal surface of the BBB, has been reported to be successful in increasing brain exposure of therapeutic molecules as well as being clinically efficacious and safe.

WO 2020/144233 discloses variable domain of camelid heavy chain-only (VHH) molecules, which bind the transferrin receptor (TfR) and uses thereof to transport molecules of pharmaceutical or diagnostic interest into cells and in organs, in pathological conditions including cancer.

WO 2016/077840, WO 2019/089395, WO 2020/056327 and WO 2022/103769 disclose TfR-specific binding moieties that can be used to carry biomolecules across membranes, including the BBB and the gastrointestinal tract. These TfR-specific binding moieties include single domain nurse shark variable domain of new antigen receptor (VNAR) antibodies that bind to TfR.

WO 2016/081643 relates to anti-transferrin receptor antibodies and methods of using the same.

There is still a need for efficacious transporters of therapeutic molecules into the brain. These transporters should be able to conjugate or fuse to a therapeutic or diagnostic molecule in a manner that does not impact the target engagement of the transporter or the therapeutic efficacy of the therapeutic molecule.

### SUMMARY

It is a general objective to provide VHH molecules specific to the transferrin receptor 1 and that can effectively transport cargo through receptor-mediated transcytosis into a desired compartment.

These and other objectives are met by embodiments of the invention.

The present invention is defined in the independent claims. Further embodiments of the invention are defined in the dependent claims.

An aspect of the invention relates to a variable domain of heavy chain-only (VHH) antibody binding specifically to a transferrin receptor 1 (TfR1). The VHH antibody comprises a complementarity determining region 1 (CDR1) having an amino acid sequence selected from the group consisting of GSIFGSKR as defined in SEQ ID NO: 1 and GSIFGFNA as defined in SEQ ID NO: 2. The VHH antibody also comprises a CDR2 having an amino acid sequence selected from the group consisting of ITYRGTT as defined in SEQ ID NO: 3 and IAVAGST as defined in SEQ ID NO: 4. The VHH antibody further comprises a CDR3 having an amino acid sequence selected from the group consisting of WMFTTDNY as defined in SEQ ID NO: 5 and WMYATANY as defined in SEQ ID NO: 6. If the CDR1 has the amino acid sequence as defined in SEQ ID NO: 2, then the CDR3 has the amino acid sequence as defined in SEQ ID NO: 6. The VHH antibody does not comprise a CDR1 having the amino acid sequence as defined in SEQ ID NO: 2, a CDR2 having the amino acid sequence as defined in SEQ ID NO: 4, and a CDR3 having the amino acid sequence as defined in SEQ ID NO: 6.

Another aspect of the invention relates to a VHH antibody binding specifically to a TfR1. The VHH antibody comprises a CDR1 consisting of the amino acid sequence X₁X₂IX₃GSKR as defined in SEQ ID NO: 7, wherein X₁ is G or E, X₂ is S, D or I, and X₃ is F or N. The VHH antibody also comprises a CDR2 consisting of the amino acid sequence ITX₄X₅GTT as defined in SEQ ID NO: 8, wherein X₄ is Y or V, and X₅ is R, H or G. The VHH antibody further comprises a CDR3 consisting of the amino acid sequence WMFTTX₆NY as defined in SEQ ID NO: 9, wherein X₆ is D, T or N.

A further aspect of the invention relates to a fusion molecule comprising a VHH antibody according above, linked to at least one molecule.

Related aspects of the invention define a fusion molecule according to above for use as a medicament, wherein the at least one molecule is a therapeutic agent, or for use in treatment of a central nervous system (CNS) disease or disorder, wherein the at least one molecule is a therapeutic agent capable of treating the CNS disease or disorder.

Yet another aspect of the invention relates to a pharmaceutical composition comprising a fusion molecule according to above and a pharmaceutically acceptable vehicle. In such a pharmaceutical composition, the at least one molecule is a therapeutic agent.

Other aspects of the invention relate to a nucleic acid molecule encoding a VHH antibody or a fusion molecule according to above, an expression vector comprising a nucleic acid molecule according to above operably linked to a promoter, and a host cell comprising a nucleic acid molecule according to above or an expression vector according to above.

The VHH antibodies of the present invention bind specifically to the TfR1 without interfering with the binding of transferrin to the TfR1. The binding properties of the VHH antibodies are tailored to be optimal for transcytosis over endothelial cells at the BBB. The VHH antibodies can thereby be used as transporters for various molecules, including therapeutic agents or diagnostic imaging agents, into the brain compartment when administered systemically.

### BRIEF DESCRIPTION OF THE DRAWINGS

The embodiments, together with further objects and advantages thereof, may best be understood by making reference to the following description taken together with the accompanying drawings, in which:

FIG. 1. Strategy for production of TfR1-binding VHHs

Graphical illustration of llama immunizations with immunogens (1A). Immunization of llama (Llama glama), N=2, was performed according to the ModiPhage™ method at Modiquest Research BV (the Netherlands). Primary immunization was performed with an ectodomain of human transferrin receptor 1 (hTfR1) (500 μg protein+Complete Freund's Adjuvant (CFA), intramuscular (i.m.)) at Day 1. Boost doses (500 μg protein+Incomplete Freund's Adjuvant (IFA), i.m.) were given on Day 21 of mouse TfR1 (mTfR1) and Day 42 of hTfR1. On Day 54, peripheral blood withdrawal was performed to study immune response with enzyme-linked immunosorbent assay (ELISA), testing for presence of both immunoglobulin G1 (IgG1) and IgG2/3 antibodies and their reactivity towards mTfR1 and hTfR1. Immunization was repeated on Day 86 (hTfR1+IFA) and Day 107 (hTfR1+IFA). On Day 117 a new peripheral blood sample was analyzed. An additional pre-harvest boost dose with a 1:1 mixture of hTfR1 and mTfR1 (250 μg+250 μg+IFA) was given on Day 120 followed by peripheral blood lymphocyte (PBL) harvest on Day 124.

Graphical overview of phage library establishment (1B). PBLs were isolated by density gradient centrifugation using Ficoll-Paque™ PLUS ˜1.5×10⁹. Ribonucleic acid (RNA) was extracted followed by reverse transcription to complimentary deoxyribonucleic acid (cDNA). cDNA was then used as a template for a polymerase chain reaction (PCR) to amplify the IgG repertoire (IgG2/3, from variable heavy (VH) domain to constant heavy chain 2 (CH2), heavy chain only antibodies). A first PCR reaction to amplify all antibodies (IgG1 and IgG2/3) and a second nested PCR reaction were performed to amplify and isolate the VHH repertoire (as described in in Pardon et al., A general protocol for the generation of Nanobodies for structural biology. Nat Protoc 9: 674-693 (2014)). Vector digestion was followed by DNA amplification as described above and a total of 1600 ng DNA was used to electroporate TG1 Escherichia coli in yielding a library of estimated size 3.2×10⁸.

FIG. 2. Graphical representations of VHH and various VHH-containing fusion proteins

FIG. 2 schematically illustrates a VHH monomer and various VHH-containing fusion proteins. Free VHHs bind as monomers to one binding site on TfR1. A bivalent VHH fusion protein was formed between two VHHs and a human fragment crystallizable (Fc) region. Functional monovalent fusion proteins were produced between one VHH and a single-chain variable fragment (scFv) or between one VHH and a non-antibody-derived molecule (X). FIG. 2 also shows a bivalent VHH fusion protein with two non-antibody-derived molecules (X).

FIG. 3. Affinity measurements of monomeric VHHs binding to hTfR1 using SPR

The figure shows representative sensorgrams of KB_A01 (3A) at increasing concentrations ranging from 6.25 to 100 nM binding to hTfR1 loaded on human transferrin (hTf) immobilized to a dextran coated gold (CM5) chip (Cytiva) by amine coupling. As a control benchmark, full-length monoclonal antibody BA1 (3B) was run at the same concentrations.

FIG. 4. Affinity measurements of dimeric VHH-Fc proteins binding to hTfR1 using SPR

The figure shows representative sensorgrams of hTfR1 at increasing concentrations ranging from 0.16 to 100 nM binding to Fc-fused KB_A01 (4A) loaded on a protein A coated chip (Cytiva). As a control benchmark, full-length monoclonal antibody BA2 (4B) and benchmark VHH-Fc BV (4C) were also tested. KB_A01, BA2 and BV were also tested against the mTfR1, where only BV bound with detectable affinity (4D).

FIG. 5. Affinity measurements of dimeric KB_A01-Fc to hTfR1 and cTfR1 in the absence and presence of hTf

The figure shows representative sensorgrams of KB_A01-Fc fusion protein binding to hTfR1 (5A-5B) or cynomolgus TfR1 (cTfR1) (5C-5D) in the absence (5A and 5C) and presence (5B and 5D) of an excess concentration of 250 nM hTf.

FIG. 6. Affinity measurements of benchmark reference antibodies to hTfR1 in the absence and presence of hTf

The figure shows representative sensorgrams of benchmark antibody BA2 (6A-6B) or benchmark antibody BA1 (6C-6D) binding to hTfR1 in the absence (6A and 6C) and presence (6B and 6D) of an excess concentration of 250 nM hTf.

FIG. 7. Sequence alignments of closely related sequences to KB_A01

The figure shows amino acid sequences of KB_A01 (SEQ ID NO: 23) and three closely related clones KB_A09 to KB_A11 in single letter code. Dots indicate amino acids identical to reference sequence (KB_A01) and boxes indicate the three complementarity-determining regions (CDRs).

FIG. 8. Binding to the apical and the extracellular domain of the hTfR1 measured by ELISA

The apical domain of hTfR1 was expressed with a M13-phage as stabilizing unit and was used to determine apical binding of KB_A01, benchmark antibody BA2 and benchmark VHH-Fc BV and lack thereof for BV-Fc (8A). Variants of KB_A01 were also assayed in this system, where representative clones confirmed binding to the apical domain (8B). A validating ELISA was performed with soluble ectodomain (no M13 phage) to representative clones or benchmark antibodies shown at 2.5 μg/mL for full length IgGs and 1.25 μg/mL for VHH-Fcs (8C).

FIG. 9. Affinity measurements of fusion proteins of VHH and scFv binding to hTfR1 using SPR

Human TfR1 was amine coupled to the surface of a CM5 chip and exposed to KB_A01 genetically fused to a scFv in the C-terminus (VHH-scFv, 9A) or in the N-terminus (scFv-VHH, 9B). Exposure at increasing concentrations ranging from 0.25 to 64 nM in 1:4 step increments.

FIG. 10. Cellular uptake via TfR1 in HEK293T cells

HEK293T cells were cultured until confluency (4-5 days) in 96 well plates. Test compounds (KB_A01-Fc, BA1, BA2 and a negative non-binding VHH-Fc, negative control) were added at 3.3-20 nM diluted in DMEM and incubated for 15 or 45 minutes and then rinsed by PBS and fixed in 4% PFA (10A, 10B). For the longer incubation time (10C), replacement of medium was performed twice; at t=30 minutes of incubation and again at t=120, with fresh DMEM. After incubation times, 15, 40-45 or 240 minutes, cells were immunostained and analyzed with confocal microscopy. Shown here are representative images of cellular uptake after 40 minutes at 20 nM (10A) for KB_A01-Fc, BA1 and a negative control, or at 3.3 nM comparing 15 and 45 minutes incubation (10B) for KB_01-Fc and BA1. Incubation of KB_A01-Fc, BA1 and BA2 as well as negative control are shown at 240 minutes as inverted grayscale (10C). Results at t=240 minutes show that BA1 has clearly less signal (intracellular presence) at this timepoint compared to BA2 and KB_A01-Fc, indicating a higher grade of intracellular degradation. FIGS. 10A and 10C show absence of signal using the non-hTfR1-binding VHH-Fc negative control. Images are shown as 8-bit grayscale (10A, 10B), or as inverted grayscale (10C). Scale bar 50 μM.

FIG. 11. Transcytosis experiments in brain-like endothelial cells in an in vitro BBB model

In vitro BBB transwell assay with brain-like endothelial cells as described in Sjöström et al., Transport study of interleukin-1 inhibitors using a human in vitro model of the blood-brain barrier, Brain Behaviour, Immunity Health 16: 100307 (2021). Test compound (500 nM in physiological buffer) was added to donor wells and compartments were harvested at 180 minutes (n=3 independent wells per sample). Cells were lysed and the contents of all compartments were analyzed with ELISA reactive to human Fc. Control IgG used was a known anti-IL1beta antibody. All test items were reactive to human Fc and were analyzed based on their own standard curve and an IgG standard on the same sample plate. KB_A01-Fc showed higher transcytosis ability than control IgG antibody and benchmark antibody BA2.

FIG. 12. PrismA™ enabling mutations of KB_A01

The figure shows KB_A01, KB_A12, and the positive control 1, all with a His-tag produced in E. coli and purified using IMAC (Ni-NTA) and run on SDS-PAGE (12A). IMAC purified material was subsequently purified using a protein A based resin (PrismA™ resin). The acid eluted fractions (12B) and flow through fraction (12C) were analyzed by SDS-PAGE. KB_A01 (12D) and KB_A12 (12E) were also tested for binding to PrismA™ on a pre-immobilized chip (Cytiva) by SPR over a concentration range from 1.95 to 500 nM.

FIG. 13. Brain and blood distribution after systemic administration of VHH-Fc fusion KB_A01 in TfR1 extracellular domain-humanized mice

Radiolabeled [¹²⁵I]VHH-Fc fusion proteins were injected as described in Example XI in human extracellular domain chimeric TfR1 (hECD-TfR1) mice with—homozygous (HOM), heterozygous (HET) and wild type (WT) genotype. Blood concentrations from blood samples taken at t=5 minutes, t=30 minutes, t=1 hour and terminally (at 2.0 hours) are shown (13A). At 2 hours after injection, animals were euthanized, transcardially perfused with NaCl and brains were excised and analyzed for radioactivity, blood compartments and brain concentrations at 2.0 hours are shown (13B, 13C). Blood concentrations are expressed as (% of injected dose, radioactivity), while brain uptake is expressed as standardized uptake value, SUV (% of injected dose corrected for animal body weight).

### DETAILED DESCRIPTION

The invention relates to variable domain of heavy chain-only (VHH) antibodies, also referred to as single-domain antibodies, and in particular to such VHH antibodies that are able to bind the transferrin (Tf) receptor 1 (TfR1), and to the use of such VHH antibodies to transport molecules across the blood-brain barrier (BBB).

Brain exposure of drugs targeting diseases of the central nervous system (CNS) is inherently difficult since the BBB protects the brain from unwanted substances present in the peripheral circulation, including antibodies, proteins and other molecules that may have a therapeutic effect within the brain. Receptor-mediated transcytosis, a natural mechanism using endogenous receptors expressed at the luminal surface of the BBB, has been suggested to increase BBB exposure of drugs. One such endogenous receptor that could be used to achieve receptor-mediated transcytosis is the transferrin receptor.

The transferrin receptor is a carrier protein for transferrin, which is a glycoprotein that binds to and mediates transport of iron (Fe) through the blood plasma. The transferrin receptor imports iron by internalizing the transferrin-iron complex through receptor-mediated endocytosis. In humans and other mammals, there are two transferrin receptors: transferrin receptor 1 (TfR1) and transferrin receptor 2 (TfR2). TfR1 is a high affinity ubiquitously expressed receptor while expression of TfR2 is restricted to certain cell types and is unaffected by intracellular iron concentrations. TfR2 binds to transferrin with a 25- to 30-fold lower affinity than what TfR1 does. Transferrin receptor as used herein refers to the TfR1 homologue, also known as Cluster of Differentiation 71 (CD71), which is encoded by the TFRC gene in humans.

Extracellular domains of human TfR1 (hTfR1) and mouse TfR1 (mTfR1) were used to immunize llama (Llama glama) animals and create a library of VHH antibody expressing clones. These VHH antibodies were screened for binding to hTfR1 and a key VHH antibody KB_A01 having desired binding properties was selected. Further VHH antibodies were produced by selected CDR modifications and by identification of VHH antibodies with sequence similarities to KB_A01. The VHH antibodies of the invention bind specifically to hTfR1 and also to cynomolgus TfR1 (cTfR1) with binding characteristics suitable for transcytosis including binding affinity, non-interference with transferrin binding site and binding to an apical domain of hTfR1. As shown in the experimental section, these VHH antibodies retain their TfR1 binding characteristics when presented as VHH-containing fusion proteins, including bivalent VHH fusion proteins formed between two VHH antibodies and human constant antibody fragment crystallizable region (Fc) and functional fusion proteins fused to a drug molecule, such as a single-chain variable fragment (scFv). Experimental data further show that the VHH antibodies, including VHH-containing fusion proteins, were taken up by human cells expressing hTfR1 and were transcytosed over a human brain-like endothelial cell monolayer used as an in vitro model of the BBB.

The VHH antibodies of the invention further have advantages as compared to VHH antibodies as disclosed in WO 2020/144233. Firstly, the VHH antibodies of the invention bind to a different epitope on hTfR1 as compared to these prior art VHH antibodies disclosed in Example VIII. The epitope, to which the prior art VHH antibodies bind, is not within the so-called apical domain of hTfR1, to which the VHH antibodies of the invention bind. Binding to the apical domain of the ectodomain of the TfR1 is advantageous as compared to binding to the helical or protease-like domains for the purpose of BBB delivery and receptor-mediated transcytosis, as these do not interfere transferrin-binding (Daniels et al., The transferrin receptor part I: Biology and targeting with cytotoxic antibodies for the treatment of cancer. Clin Immunol. 2006; 121: 144-158 (2006); WO 2016/081643). Furthermore, binding of the VHH antibodies of the invention to the apical domain of TfR1 does not interfere with binding of holo-transferrin to the TfR1, see FIG. 5, and Table 5. Another significant advantage of the VHH antibodies of the invention as compared to the VHH antibodies as disclosed in WO 2020/144233 is that the present VHH antibodies have binding characteristics in terms of affinity range to hTfR1 and in terms of its release from hTfR1 that allow for efficient BBB crossing and a high relative uptake in the brain. The VHH antibodies as disclosed in WO 2020/144233 have a significantly higher affinity to hTfR1 making them vulnerable for lysosomal degradation when endocytosed in the endothelial cells of the BBB rather than crossing the BBB. The VHH antibodies of WO 2020/144233 thereby may become trapped in the endothelial cells and eventually degraded therein. Another significant advantage of the VHH antibodies of the invention as compared to VHH antibodies as disclosed in WO 2020/144233 is that there is no significant change in affinity of the VHH antibodies of the invention to hTfR1 in the presence or absence of human transferrin (hTf). However, the affinity of VHH antibodies as disclosed in WO 2020/144233 to hTfR1 was significantly reduced in the presence of hTf as compared in the absence of hTf as shown in Table 5. This dependency of the affinity to hTfR1 on the concentration of hTf makes it hard to select suitable amounts of the VHH antibodies needed to achieve a desired receptor-mediated transcytosis across the BBB.

An aspect of the invention therefore relates to a variable domain of heavy chain-only (VHH) antibody binding specifically to a transferrin receptor 1 (TfR1). The VHH antibody comprises a complementarity determining region 1 (CDR1) having an amino acid sequence selected from the group consisting of GSIFGSKR as defined in SEQ ID NO: 1 and GSIFGFNA as defined in SEQ ID NO: 2. The VHH antibody also comprises a CDR2 having an amino acid sequence selected from the group consisting of ITYRGTT as defined in SEQ ID NO: 3 and IAVAGST as defined in SEQ ID NO: 4. The VHH antibody further comprises a CDR3 having an amino acid sequence selected from the group consisting of WMFTTDNY as defined in SEQ ID NO: 5 and WMYATANY as defined in SEQ ID NO: 6. According to the invention, if the CDR1 has the amino acid sequence as defined in SEQ ID NO: 2, then the CDR3 has the amino acid sequence as defined in SEQ ID NO: 6. However, the VHH antibody does not comprise a CDR1 having the amino acid sequence as defined in SEQ ID NO: 2, a CDR2 having the amino acid sequence as defined in SEQ ID NO: 4, and a CDR3 having the amino acid sequence as defined in SEQ ID NO: 6.

The VHH antibodies of this aspect are generated based on CDR shuffling between KB_A01 and a VHH antibody containing several alanine mutations in the CDRs as compared to KB_A01. The strategy was to treat the VHH antibody containing alanine mutations in the CDRs, denoted KB_ref herein, as a naturally occurring alanine scan but replace entire CDRs rather than single amino acid residues in KB_A01. KB_ref was initially identified as a hTfR1 binder when analyzed in ELISA. However, this VHH antibody KB_ref unexpectedly did not bind hTfR1 when immobilized by protein A and analyzed using surface plasmon response (SPR). However, several VHH antibodies, KB_A03, KB_A04, KB_A06 and KB_A07, obtained by selected CDR shuffling were indeed capable of binding VHH with desired binding characteristics. This was highly unexpected given that the VHH antibody KB_ref, from which some of the CDRs in KB_A03, KB_A04, KB_A06 and KB_A07 were derived, was not able to bind hTfR1.

In more detail, the scaffold VHH antibody lacking alanine residues in the CDRs, i.e., KB_A01, has CDR1 as defined in SEQ ID NO: 1, CDR2 as defined in SEQ ID NO: 3 and CDR3 as defined in SEQ ID NO: 5, whereas the VHH antibody containing alanine mutations in the CDRs, i.e., KB_ref, has CDR1 as defined in SEQ ID NO: 2, CDR2 as defined in SEQ ID NO: 4 and CDR3 as defined in SEQ ID NO: 6.

Replacing all CDRs of KB_A01 with the CDRs of KB_ref resulted in loss of binding to hTfR1, KB_A08 as shown in Table 7. Hence, the VHH antibodies of this aspect does not comprise a CDR1 having the amino acid sequence as defined in SEQ ID NO: 2, a CDR2 having the amino acid sequence as defined in SEQ ID NO: 4, and a CDR3 having the amino acid sequence as defined in SEQ ID NO: 6. Furthermore, when replacing CDR1 of KB_A01 with CDR1 of KB_ref hTfR1 binding was lost unless also CDR3 of KB_A01 was replaced by CDR3 of KB_ref, see KB_A02 and KB_A05 in Table 5. Hence, if the CDR1 of the VHH antibodies of this aspect has the amino acid sequence as defined in SEQ ID NO: 2, then the CDR3 has the amino acid sequence as defined in SEQ ID NO: 6.

Table 6 summarizes the CDRs of the VHH antibodies KB_A01 to KB_A08, whereas Table 7 shows binding kinetics of these VHH antibodies in the form of dimeric VHH-Fc fusion proteins to hTfR1.

In an embodiment, the VHH antibody does not comprise a CDR1 having the amino acid sequence as defined in SEQ ID NO: 2, a CDR2 having the amino acid sequence as defined in SEQ ID NO: 3 or 4, and a CDR3 having the amino acid sequence as defined in SEQ ID NO: 6. In this embodiment, the VHH antibody does not comprise a CDR1 having the amino acid sequence as defined in SEQ ID NO: 2, a CDR2 having the amino acid sequence as defined in SEQ ID NO: 3, and a CDR3 having the amino acid sequence as defined in SEQ ID NO: 6. Further, the VHH antibody does not comprise, in this particular embodiment, a CDR1 having the amino acid sequence as defined in SEQ ID NO: 2, a CDR2 having the amino acid sequence as defined in SEQ ID NO: 4, and a CDR3 having the amino acid sequence as defined in SEQ ID NO: 6.

In an embodiment, the CDR1 has the amino acid sequence as defined in SEQ ID NO: 1, the CDR2 has the amino acid sequence selected from the group consisting of SEQ ID NO: 3 and 4, and the CDR3 has the amino acid sequence as defined in SEQ ID NO: 5.

In a particular embodiment, the CDR1 has the amino acid sequence as defined in SEQ ID NO: 1, the CDR2 has the amino acid sequence as defined in SEQ ID NO: 4, and the CDR3 has the amino acid sequence as defined in SEQ ID NO: 5.

In another particular embodiment, the CDR1 has the amino acid sequence as defined in SEQ ID NO: 1, the CDR2 has the amino acid sequence as defined in SEQ ID NO: 3, and the CDR3 has the amino acid sequence as defined in SEQ ID NO: 5.

In another embodiment, the CDR1 has the amino acid sequence selected from the group consisting of SEQ ID NO: 1 and 2, the CDR2 has the amino acid sequence as defined in SEQ ID NO: 3, and the CDR3 has the amino acid sequence as defined in SEQ ID NO: 6.

In a particular embodiment, the CDR1 has the amino acid sequence as defined in SEQ ID NO: 1, the CDR2 has the amino acid sequence as defined in SEQ ID NO: 3, and the CDR3 has the amino acid sequence as defined in SEQ ID NO: 6.

In another particular embodiment, the CDR1 has the amino acid sequence as defined in SEQ ID NO: 2, the CDR2 has the amino acid sequence as defined in SEQ ID NO: 3, and the CDR3 has the amino acid sequence as defined in SEQ ID NO: 6.

In a further embodiment, the CDR1 has the amino acid sequence as defined in SEQ ID NO: 1, the CDR2 has the amino acid sequence as defined in SEQ ID NO: 4, and the CDR3 has the amino acid sequence as defined in SEQ ID NO: 6.

In an embodiment, the VHH antibody is of a formula: framework region 1 (FR1)-CDR1-FR2-CDR2-FR3-CDR3-FR4.

In an embodiment, the FR1 has an amino acid sequence QVQLQESGGGSVQAGGSLSLSCAAS as defined in SEQ ID NO: 57.

In an embodiment, the FR2 has an amino acid sequence MGWFRQAPGEQRDVVAT as defined in SEQ ID NO: 58.

In an embodiment, the FR3 has an amino acid sequence EYADSVKGRFTISRDNAKNTVYLQMNNLKPEDTAVYYC as defined in SEQ ID NO: 59.

In an embodiment, the FR4 has an amino acid sequence WGQGTQVTVSS as defined in SEQ ID NO: 22.

In a particular embodiment, the VHH antibody has amino acid sequences of FR1, FR2, FR3 and FR4 as defined in SEQ ID NO: 57, 58, 59 and 22.

In an embodiment, the VHH antibody has an amino acid sequence as defined in SEQ ID NO: 23. Such a VHH antibody is denoted KB_A01 herein.

In another embodiment, the VHH antibody has an amino acid sequence as defined in SEQ ID NO: 24. Such a VHH antibody is denoted KB_A03 herein.

In a further embodiment, the VHH antibody has an amino acid sequence as defined in SEQ ID NO: 25. Such a VHH antibody is denoted KB_A04 herein.

In an additional embodiment, the VHH has an amino acid sequence as defined in SEQ ID NO: 44. Such a VHH antibody is denoted KB_A06 herein.

In yet another embodiment, the VHH antibody has an amino acid sequence as defined in SEQ ID NO: 26. Such a VHH antibody is denoted KB_A07 herein.

In an embodiment, the VHH antibody has an amino acid sequence selected from the group consisting of SEQ ID NO: 23 to 26, 44, preferably selected from the group consisting of SEQ ID NO: 23 to 26.

Another aspect of the invention relates to a VHH antibody binding specifically to a TfR1. The VHH antibody comprises a CDR1 consisting of the amino acid sequence X₁X₂₁X₃GSKR as defined in SEQ ID NO: 7, wherein X₁ is G or E, X₂ is S, D or I, and X₃ is F or N. The VHH antibody also comprises a CDR2 consisting of the amino acid sequence ITX₄X₅GTT as defined in SEQ ID NO: 8, wherein X₄ is Y or V, and X₅ is R, H or G. The VHH antibody further comprises a CDR3 consisting of the amino acid sequence WMFTTX₆NY as defined in SEQ ID NO: 9, wherein X₆ is D, T or N.

This aspect of the invention relates a family of VHH antibodies having closely related CDR regions and all binding to hTfR1 with high affinity.

In an embodiment, the CDR1 consists of the amino acid sequence X₁X₂₁X₃GSKR as defined in SEQ ID NO: 7, wherein X₁ is G or E, X₂ is D or I, and X₃ is F or N. In this embodiment, the CDR2 consists of the amino acid sequence ITX₄X₅GTT as defined in SEQ ID NO: 8, wherein X₄ is Y or V, and X₅ is R, H or G. Furthermore, the CDR3 consists of the amino acid sequence WMFTTX₆NY as defined in SEQ ID NO: 9, wherein X₆ is D, T or N.

In a particular embodiment, the CDR1 consists of the amino acid sequence GDIX₃GSKR as defined in SEQ ID NO: 11, wherein X₃ is F or N. In this particular embodiment, the CDR2 consists of the amino acid sequence ITVX₅GTT as defined in SEQ ID NO: 12, wherein X₅ is R or G. Furthermore, the CDR3 consists of the amino acid sequence WMFTTX₆NY as defined in SEQ ID NO: 9, wherein X₆ is T or N.

In a preferred embodiment, the CDR1 consists of the amino acid sequence GDINGSKR as defined in SEQ ID NO: 13, the CDR2 consists of the amino acid sequence ITVRGTT as defined in SEQ ID NO: 14, and the CDR3 consists of the amino acid sequence WMFTTTNY as defined in SEQ ID NO: 10.

In another preferred embodiment, the CDR1 consists of the amino acid sequence GDIFGSKR as defined in SEQ ID NO: 15, the CDR2 consists of the amino acid sequence ITVGGTT as defined in SEQ ID NO: 16, and the CDR3 consists of the amino acid sequence WMFTTNNY as defined in SEQ ID NO: 55.

In a further preferred embodiment, the CDR1 consists of the amino acid sequence EIINFGSKR as defined in SEQ ID NO: 17, the CDR2 consists of the amino acid sequence ITYHGTT as defined in SEQ ID NO: 18, and the CDR3 consists of the amino acid sequence WMFTTDNY as defined in SEQ ID NO: 5.

In an embodiment, the VHH antibody is of formula: FR1-CDR1-FR2-CDR2-FR3-CDR3-FR4.

In an embodiment, the FR1 has an amino acid sequence QVQLQESGGGX₇VQAGGSLX₈LSCAAS as defined in SEQ ID NO: 19, wherein X₇ is S or L, and Xa is S or R.

In an embodiment, the FR2 has an amino acid sequence MGWFRQAPGX₉X₁₀RDX₁₁VAT as defined in SEQ ID NO: 20, wherein X₉ is E, K or Q, X₁₀ is Q or A, and X₁₁ is V or L.

In an embodiment, the FR3 has an amino acid sequence X₁₂YX₁₃DSVKGRFTISRDNAX₁₄NTVYLQMNX₁₅LKPEDTAX₁₆YYC as defined in SEQ ID NO: 21, wherein X₁₂ is E or K, X₁₃ is A or E, X₁₄ is K or N, X₁₅ is N or S, and X₁₆ is V or F.

In an embodiment, the FR4 has an amino acid sequence WGQGTQVTVSS as defined in SEQ ID NO: 22.

In a particular embodiment, the VHH antibody has amino acid sequences of FR1, FR2, FR3 and FR4 as defined in SEQ ID NO: 19, 20, 21 and 22.

The present invention also encompasses that the VHH antibody has an amino acid sequence of FR1 comprising or consisting of SEQ ID NO: 19 or 57, or a variant thereof having at least 88% sequence identity to SEQ ID NO: 19 or 57, preferably at least 92% sequence identity, and more preferably at least 96% sequence identity.

The present invention also encompasses that the VHH antibody has an amino acid sequence of FR2 comprising or consisting of SEQ ID NO: 20 or 58, or a variant thereof having at least 82% sequence identity to SEQ ID NO: 20 or 58, preferably at least 88% sequence identity, and more preferably at least 94% sequence identity.

The present invention also encompasses that the VHH antibody has an amino acid sequence of FR3 comprising or consisting of SEQ ID NO: 21 or 59, or a variant thereof having at least 92% sequence identity to SEQ ID NO: 21 or 59, preferably at least 94% sequence identity, and more preferably at least 97% sequence identity.

The present invention also encompasses that the VHH antibody has an amino acid sequence of FR4 comprising or consisting of SEQ ID NO: 22, or a variant thereof having at least 72% sequence identity to SEQ ID NO: 22, preferably at least 81% sequence identity, and more preferably at least 90% sequence identity.

As used herein, the term “% sequence identity” may be determined using methods well known in the art. For example, % sequence identity be calculated as follows. The query sequence is aligned to the target sequence using the CLUSTAL W algorithm. A comparison is made over the window corresponding to the shortest of the aligned sequences. The shortest of the aligned sequences may in some instances be the target sequence. In other instances, the query sequence may constitute the shortest of the aligned sequences. The amino acid residues at each position are compared and the percentage of positions in the query sequence that have identical correspondences in the target sequence is reported as % sequence identity.

An amino acid sequence having a defined % sequence identity of a reference amino acid sequence is preferably obtained by amino acid substitutions, such as by conservative amino acid replacements. Conservative amino acid replacements, also denoted as conservative amino acid substitutions or mutations, is an amino acid replacement in an amino acid sequence that changes a given amino acid to a different amino acid with similar biochemical, structural and/or chemical properties.

For example, amino acids may be sorted into six main classes on the basis of their structure and the general chemical characteristics of their side chains (R groups):
Aliphatic: Isoleucine (I), Leucine (L), Glycine (G), Alanine (A), Valine (V);
Hydroxyl or sulfur/selenium-containing: Serine (S), Cysteine (C), Threonine (T), Methionine (M);
Cyclic: Proline (P)
Aromatic: Phenylalanine (F), Tyrosine (Y), Tryptophan (W);
Basic: Histidine (H), Lysine (K), Arginine (R); and
Acidic and their amides: Aspartate (D), Glutamate (E), Asparagine (N), Glutamine (Q).

This means that an amino acid sequence having a defined % sequence identity of a reference amino acid sequence is preferably obtained by one or more conservative amino acid replacements of one or more amino acid residues in the reference amino acid sequence with a respective amino acid from the same R group listed above as the given amino acid residue.

In an embodiment, the VHH antibody has an amino acid sequence as defined in SEQ ID NO: 27. Such a VHH antibody is denoted KB_A09 herein.

In another embodiment, the VHH antibody has an amino acid sequence as defined in SEQ ID NO: 28. Such a VHH antibody is denoted KB_A10 herein.

In a further embodiment, the VHH antibody has an amino acid sequence as defined in SEQ ID NO: 29. Such a VHH antibody is denoted KB_A11 herein.

In yet another embodiment, the VHH antibody has an amino acid sequence as defined in SEQ ID NO: 23. Such a VHH antibody is denoted KB_A01 herein.

FIG. 7 shows a sequence alignment of closely related VHH antibodies KB_A01, KB_A09, KB_A10 and KB_A11. Table 7 shows binding kinetics of the VHH antibodies KB_A09, KB_A10 and KB_A11 in the form of dimeric VHH-Fc fusion proteins to hTfR1.

In an embodiment, the VHH antibody has an amino acid sequence selected from the group consisting of 23, 27 to 29, preferably selected from the group consisting of SEQ ID NO: 27 to 29.

In an embodiment, the VHH antibody has an amino acid sequence selected from the group consisting of SEQ ID NO: 23-29, 44. In a preferred embodiment, the VHH has an amino acid sequence selected from the group consisting of SEQ ID NO: 23-29.

The VHH antibodies of the invention bind specifically to a TfR1.

By “bind specifically to” and similar expressions is meant that the molecule in question, such as an VHH antibody, specifically binds to the target antigen without any significant binding to other molecules. The specificity of an antibody can be determined based on affinity and/or avidity. The affinity, represented by the equilibrium dissociation constant of an antigen with the antibody (K_{D}) is a measure for the binding strength between an antigenic determinant, i.e., epitope, and an antigen-binding site on the antibody. The lower the value of K_{D}, the stronger the binding strength between the antigenic determinant and the antibody. Alternatively, the affinity can also be expressed as the equilibrium association constant (K_{A}), which is 1/K_{D}. As will be clear to the skilled person, affinity can be determined in a manner known per se, depending on the specific antigen of interest.

Typically, antibodies will bind to their antigen with an equilibrium dissociation constant (K_{D}) of 10⁻⁵ to 10⁻¹² moles/liter (M) or less, and preferably 10⁻⁷ to 10⁻¹² M or less and more preferably 10⁻⁸ to 10⁻¹² M, i.e., with an affinity constant (K_{A}) of 10⁵ to 10¹² M⁻¹ or more, and preferably 10⁷ to 10¹² M⁻¹ or more and more preferably 10⁸ to 10¹² M⁻¹. Generally, any K_{D} value greater than 10⁻⁴ M (or any K_{A} value lower than 10⁴ M⁻¹) is considered to indicate non-specific binding.

Specific binding of an antibody to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, surface plasmon resonance (SPR), biolayer interferometry (BLI) and different variants thereof known per se in the art.

The affinity of VHH antibodies to TfR1 should be tailored to be within a specific range in order to achieve an efficient BBB crossing and a high relative uptake in the brain. Generally, if the affinity of antibodies to TfR1 is too low, such as a K_{D} larger than about 1 μM the antibodies have too low affinity for efficient binding to TfR1 and uptake by the endothelial cells of the BBB. This means that a large portion of the antibodies will remain in the peripheral blood system. Correspondingly, if the antibodies have too high affinity, such as K_{D} significantly lower than 1 nM (<<1 nM), the antibodies are endocytosed but are instead subject to lysosomal degradation in the endothelial cells of the BBB. This means that the VHH antibodies, for optimal receptor-mediated endocytosis, i.e., receptor-mediated cellular uptake, and transcytosis, i.e., transport across the interior of a cell, should have an affinity (K_{D}) in the low nM to the nM range (Bien-Ly N, et al., Transferrin receptor (TfR) trafficking determines brain uptake of TfR antibody affinity variants. J Exp Med. 211(2):233-244 (2014), WO 2016/081643).

As is shown in Table 2, the VHH antibody KB_A01 of the invention has, in monomeric form, an affinity (K_{D}) of 4.1 nM, i.e., in the nM range. Furthermore, the affinity still remains in the desired low nM to nM range when included in a VHH-containing fusion protein (see FIG. 2), such as a dimeric-VHH Fc fusion protein with a K_{D} of 0.18 nM, see Table 4, or a fusion with a scFv with a K_{D} of 3.2-3.4 nM, see Table 9. Also the other VHH antibodies of the invention have affinities to TfR1 in the low nM to nM range and are thereby suitable for BBB-crossing by receptor-mediated endocytosis and transcytosis, see Table 7 with K_{D} ranging from 0.15 to 5.1 nM as dimeric-VHH Fc fusion proteins.

In an embodiment, the VHH antibodies of the invention bind specifically to human TfR1 (hTfR1).

In an embodiment, the VHH antibodies of the invention bind specifically to hTfR1 with an affinity (K_{D}) selected within a range of from 0.1 to 150 nM, preferably within a range of from 0.1 to 100 nM. In a particular embodiment, the VHH antibodies of the invention, in monovalent form, bind specifically to hTfR1 with K_{D} selected within a range of from 1 to 150 nM, preferably within a range of from 1 to 100 nM, and more preferably within a range of from 1 to 50 nM.

In an embodiment, the VHH antibodies of the invention bind specifically to cTfR1. In a particular embodiment, the VHH antibodies of the invention bind not only specifically to hTfR1 but also to cTfR1, see Table 5.

In an embodiment, the VHH antibodies of the invention do not bind specifically to mTfR1.

Structurally, the hTfR1 is a dimeric transmembrane glycoprotein with a large ectodomain (residues 90-760), an intramembranous region (residues 62-89) and the remaining 61 residues in the cytoplasm. The ectodomain has three domains, the helical domain (residues 606-760), the protease-like domain (residues 121-183, 384-605) and the apical domain (residues 184-383). The helical domain is responsible for receptor dimerization. Transferrin binds to the helical and protease-like domains.

In an embodiment, the VHH antibodies of the invention bind specifically to the apical domain of TfR1, such as hTfR1. Binding to the apical domain of the ectodomain of the TfR1 is advantageous as compared to binding to the helical or protease-like domains for the purpose of BBB delivery and receptor-mediated transcytosis, as these do not interfere transferrin-binding (Daniels et al., The transferrin receptor part I: Biology and targeting with cytotoxic antibodies for the treatment of cancer. Clin Immunol. 2006; 121: 144-158 (2006); WO 2016/081643). Furthermore, binding of the VHH antibodies of the invention to the apical domain of TfR1 does not interfere with binding of holo-transferrin to the TfR1, see FIG. 5, and Table 5. Hence, the VHH antibodies of the invention do not interfere with the transferrin-binding for iron uptake by cells using the TfR1. Accordingly, it is preferred, in terms of transcytosis capacity, if the VHH antibodies bind to the apical domain of TfR1.

In an embodiment, the VHH antibodies of the invention are camelid VHH antibodies.

In another embodiment, the VHH antibodies of the invention are humanized VHH antibodies. For instance, the CDR regions of the VHH antibodies may be grafted onto a human backbone. Soler et al., Effect of Humanizing Mutations on the Stability of the Llama Single-Domain Variable Region, Biomolecules 11(2): 163 (2021) identified several amino acid positions and the N-terminal Gln as hot sports for converting camelid VHHs to human consensus germ-line sequence.

Here below, sequence alignments are presented between the framework regions of KB_A01, a reference human VH (sVH), a universal VHH (uVHH) and a humanized VHH sequence (hVHH) as disclosed in the above-mentioned Biomolecules article.

| | |
|---|---|
| QVQLQESGGGSVQAGGSLSLSCAAS | KB_A01 FR1 |
| ----VQ----L--P----R------ | sVH FR1 |
| ----V--------P----R---T-- | uVHH FR1 |
| ----V-----L--P----R------ | hVHH FR1 |
| MGWFRQAPGEQRDVVAT | KB_A01 FR2 |
| -S-V-----KGLEW-SP | sVH FR2 |
| L--------QE-EA--A | uVHH FR2 |
| L--------QGLEA--A | hVHH FR2 |
| EYADSVKGRFTISRDNAKNTVYLQMNNLKPEDTAVYYC | KB_A01 FR3 |
| Y---------------S---L-----T--RA------- | sVH FR3 |
| Y--------------------T------------I--- | uVHH FR3 |
| Y---------------S---L-----S--RA------- | hVHH FR3 |
| WGQGTQVTVSS | KB_A01 FR4 |
| -----M----- | sVH FR4 |
| ----------- | UVHH FR4 |
| -----L----- | sVH FR4 |

The VHH antibodies of the invention can be humanized by modifications, e.g., amino acid substitutions, in FR1, FR2, FR3 and/or FR4. Amino acid positions in FR1-FR4 referred to herein are to the amino acid positions in KB_A01 (SEQ ID NO: 23) as shown in FIG. 7.

As an example, humanized positions in FR1 could be selected from E1 or Q1; V5; Q6 or E6; S11 or L11; P14; and/or R19. In an embodiment, any one of these humanized positions in FR1 are used for the humanized FR1 or any combination of two up to all of these positions are humanized. As an example, a humanized FR1 could be based on SEQ ID NO: 57 but have Q1 replaced by E, Q5 replaced by V, S19 replaced by R, or two or all of these amino acid replacements.

Humanized positions in FR2 could be selected from L34 or M34 (first amino acid position in FR2), S35 or G35 (second amino acid position in FR2), V37 or F37 (fourth amino acid position in FR2), Q43 or K43 (tenth amino acid position in FR2), G44 (eleventh amino acid position in FR2), L45 (twelfth amino acid position in FR2), E46 (13^{th} amino acid position in FR2), W47 or A47 (14^{th} amino acid position in FR2), S49 (16^{th} amino acid position in FR2) and/or P50, A50, V50 or G50 (17^{th} amino acid position in FR2). In an embodiment, any one of these humanized positions in FR2 are used for the humanized FR2 or any combination of two up to all of these positions are humanized. As illustrative example, the humanized positions in FR2 could be V37, G44, L45 and W47 or F37, G44, L45 and A47.

Humanized positions in FR3 could be selected from Y58 (1^{st} amino position in FR3), S74 (17^{th} amino acid position in FR3), K75 (18^{th} amino acid position in FR3), N76 (19^{th} amino acid position in FR3), L78 or 178 (21^{st} amino acid position in FR3), N84, S84 or T84 (27^{th} amino acid position in FR3), R86 (29^{th} amino acid position in FR3), A87 (30^{th} amino acid position in FR3)), A96 (39^{th} amino acid position in FR3) and/or R97 or A97 (40^{th} amino acid position in FR3). In an embodiment, any one of these humanized positions in FR3 are used for the humanized FR3 or any combination of two up to all of these positions are humanized.

Humanized positions in FR4 could be L109 or M109 (sixth amino acid position in FR 4).

I78 also has stabilizing effect for VHHs. Hence, Ile at position 78 stabilizes the VHH.

Example XIV herein produced and tested various humanized VHH antibodies based on the FR regions of the VHH antibody KB_A01. Positions indicated below as X could be successfully mutated to humanize the VHH antibody KB_A01 and still maintain the binding to hTfR1.

FR1 (SEQ ID NO: 78):
X₁VQLX₂ESGGGX₃VQX₄GGSLX₅LSCAAS
FR2 (SEQ ID NO: 79):
MGWFRQAPGX₆X₇X₈X₉VVAT
FR3 (SEQ ID NO: 80):
EYADSVKGRFTISRDNX₁₀KNTX₁₁YLQMNX₁₂LX₁₃PEDTAVYYC
FR4 (SEQ ID NO: 81):
WGQGTX₁₄VTVSS

The wildtype FR regions of KB_A01 has X₁=Q, X₂=Q, X₃=S, X₄=A, X₅=S, X₆=E, X₇=Q, X₈=R, X₉=D, X₁₀=A, X₁₁=V, X₁₂=N, X₁₃=K, and X₁₄=Q.

Examples of humanized VHH antibodies of the invention include VHH antibodies having FR1-FR4 regions according to above (SEQ ID NO: 78-81) include VHH antibodies having at least one of X₁-X₁₄ according to below and any remaining non-mutated one(s) of X₁-X₁₄ according to the wildtype FR regions of KB_A01 listed above:

X₁ = E, X₂ = V, X₃ = L, X₄ = P, X₅ = R, X₆ = K,
X₇ = G, X₈ = L, X₉ = E, X₁₀ = S, X₁₁ = I, X₁₂ = S,
X₁₃ = R, and X₁₄ = L.

In an embodiment, the VHH antibodies of the invention bind to protein A. In a particular embodiment, the VHH antibodies of the invention bind to protein A based resins, such as protein A chromatography resins, such as MabSelect PrismA™ resin.

Protein A is a 42 kDa surface protein originally found in the cell wall of the bacteria Staphylococcus aureus. It is encoded by the spa gene. It has found use in biochemical research because of its ability to bind immunoglobulins. It is composed of five homologous Ig-binding domains each folded into a three-helix bundle. Each domain is able to bind proteins from many mammalian species, most notably IgGs. It binds the heavy chain within the Fc region of most immunoglobulins and also within the Fab region in the case of the human VH3 family. Protein A generally does not bind or merely bind weakly to camelid VHH antibodies.

Henry K A, et al., A Rational Engineering Strategy for Designing Protein A-Binding Camelid Single-Domain Antibodies, PLoS One 11(9): e0163113 (2016) discloses how to make non-protein A binding VHH antibodies capable of binding to protein A, see Table 5.

In more detail, amino acid residue 15 should be G or D, amino acid residue 17 should be S or A, amino acid residue 19 should be R. The consensus FR1 sequence in SEQ ID NO: 19 is a protein A binding FR1 region if X₈ is R. In a preferred embodiment, the FR1 region of KB_A01 is modified by replacing S at amino acid residue 19 with R to get the protein A binding FR1 sequence presented here below and in SEQ ID NO: 82.

| | |
|---|---|
| QVQLQESGGGSVQAGGSLSLSCAAS | KB_A01 FR1 |
| QVQLQESGGGXVQAGGSLXLSCAAS | Consensus FR1 |
| ------------------R------ | Protein A binding FR1 |

Furthermore, amino acid residue 59 should be Y, amino acid residue 64 should be K or E, amino acid residue 65 should be G, amino acid residue 66 should be R, amino acid residue 68 should be T or A, amino acid residue 70 should be S, amino acid residue 75 should be A, E, K, Q or R, amino acid residue 81 should be Q, amino acid residue 83 should be N, and amino acid residue 84 should be S, N or G. The FR3 region of KB_A01 is protein A binding without any amino acid replacements. However, in an optional embodiment, the N at amino acid residue 84 could be replaced by S. The consensus FR3 sequence in SEQ ID NO: 21 is a protein A binding FR3 region if X₁₄ is A, E, K, Q or R and if X₁₅ is S, N or G.

| | |
|---|---|
| EYADSVKGRFTISRDNAKNTVYLQMNNLKPEDTAVYYC | KB_A01 FR3 |
| XYXDSVKGRFTISRDNAXNTVYLQMNXLKPEDTAXYYC | Consensus FR3 |
| -----------------K--------S----------- | Protein A binding FR3 |

In an embodiment, the FR1 region of KB_A01 is modified by replacing S at amino acid residue 19 with R and the FR3 region of KB_A01 is modified by replacing N at amino acid residue 84 with S.

In an embodiment, the VHH antibody has a FR1 region according to QVQLQESGGGSVQAGGSLRLSCAAS as defined in SEQ ID NO: 82 and a FR3 3 according to EYADSVKGRFTISRDNAKNTVYLQMNSLKPEDTAVYYC as defined in SEQ ID NO: 83.

In an embodiment, the VHH antibody of the invention is an isolated VHH antibody.

The term “isolated” when used in connection with VHH antibodies, such as in the expression “isolated VHH antibody” and the like, means the VHH antibody has been purified and removed from its original environment. An isolated VHH antibody, as used herein, is intended to refer to a VHH antibody that is substantially free of other antibodies having different antigenic specificities, e.g., an isolated VHH antibody that specifically binds TfR1, in particular human TfR1 (hTfR1), is substantially free of antibodies that specifically bind antigens other than TfR1. An isolated VHH antibody that specifically binds hTfR1 may, however, have cross-reactivity to other antigens, such as TfR1 molecules from other species, such as cTfR1. Moreover, an isolated VHH antibody may be substantially free of other cellular material and/or chemicals. For example, the isolated VHH antibody may be purified to greater than 95% or 99% purity as determined by, for example, electrophoretic, e.g., sodium dodecyl sulfate-polyacrylamide gel electrophoresis (SDS-PAGE), isoelectric focusing (IEF), capillary electrophoresis, or chromatographic methods, e.g., ion exchange or reverse-phase high-performance liquid chromatography (HPLC). The skilled person will appreciate that isolated VHH antibodies are referred to herein even though the word “isolated” is not explicitly mentioned each time the term “VHH antibodies” and similar expressions are used.

A further aspect of the embodiments includes a nucleic acid molecule encoding a VHH antibody according to the embodiments or a fusion molecule according to the embodiments, see further below. Nucleic acid molecule as used herein includes polynucleotide, oligonucleotide, and nucleic acid sequence, and generally means a polymer of DNA or RNA, which may be single-stranded or double-stranded, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. The term “nucleic acid molecule” also include complementary DNA (cDNA) and messenger RNA (mRNA).

In an embodiment, the nucleic acid molecule is an isolated nucleic acid molecule encoding a VHH antibody according to the embodiments or a fusion molecule according to the embodiments.

The nucleic acid molecules may encode a single VHH antibody according to the embodiments, multiple copies of a single VHH antibody according to the embodiments, or one or more copies of different VHH antibodies according to the embodiments.

The nucleic acid molecule may also encode other molecules than the VHH antibody of the embodiments, for instance the VHH antibody genetically fused to another molecule, i.e., in the form of a VHH-containing fusion protein as further described herein.

Another aspect of the embodiments relates to a vector comprising a nucleic acid molecule according to the embodiments.

The vector is preferably an expression vector, i.e., a vector comprising at least one nucleic acid molecule comprising coding sequences that can be expressed, such as transcribed and translated, in a host cell comprising the expression vector. The expression vector therefore comprises a nucleic acid molecule according to the embodiments operative linked to a promoter.

Operatively linked as used herein means that the nucleic acid molecule is in a correct functional location and/or orientation in relation to the promoter to enable expression of the nucleic acid molecule in the host cell, i.e., the promoter (constitutively or inducibly) controls transcription of the nucleic acid molecule.

The expression vector is in an embodiment selected among DNA molecules, RNA molecules, plasmids, episomal plasmids and virus vectors. In such an embodiment, the expression vector comprises, in addition to the nucleic acid sequence encoding the VHH antibody, control sequences needed to produce the VHH antibody in the host cell. For instance, the nucleic acid sequence encoding the VHH antibody is under transcriptional control of a promoter sequence comprised in the expression vector. A promoter is a sequence of DNA, to which proteins bind that initiate transcription of an RNA molecule from the DNA (gene) downstream of it. The promoter is preferably selected based on the particular host cell, in which the VHH antibody is to be expressed, such as the T5, T7, lac, BL21 for expression of the VHH antibody in bacterial cells, GAL1, MET25, CUP1, LAC4, ADH2, SUC2, GAPDH for expression of the VHH antibody in yeast and EF1α, CMV or CAG promoter for expression in mammalian cells, such as human cells. The promoter could be a constitutive promoter or an inducible promoter. A constitutive promoter, also referred to as constitutively active promoter, is active in all circumstances in the host cell. An inducible promoter, also referred to as inducibly active promoter, is regulated and becomes active in the host cell only in response to specific stimuli, such as a chemically inducible promoter, a temperature inducible promoter, or a light inducible promoter. The expression vector may optionally comprise other control sequences, such as an enhancer. An enhancer is a short region of DNA that can be bound by activators to increase the likelihood that transcription of a particular gene will occur. An optional signal peptide could be provided at the N-terminus or the C-terminus of the polypeptide encoded by the expression vector.

A further aspect of the embodiments relates to a host cell comprising a nucleic acid molecule or an expression vector according to the embodiments.

The nucleic acid molecule or expression vector can then be transcribed in the host cell to produce the VHH antibody in the cell.

In an embodiment, the cell is selected from the group consisting of a bacterial cell, a yeast cell, and a mammalian cell.

Yet another aspect of the embodiments relates to a VHH antibody according to the invention linked to at least one molecule. Hence, this aspect of the invention relates to a fusion molecule or protein between the VHH antibody and the at least one other molecule. The fusion molecule or protein is preferably in the form a genetic fusion between the VHH antibody and the at least one molecule. In such a case, the above-mentioned nucleic acid molecule encodes not only the VHH antibody but also at least one other molecule. For instance, the nucleic acid molecule could include from a 5′ end to a 3′ end a nucleic acid sequence encoding the VHH molecule and a nucleic acid sequence encoding the at least one other molecule or a nucleic acid sequence encoding the at least one other molecule and the nucleic acid sequence encoding the VHH molecule. Hence, the VHH molecule can be covalently linked to the at least one other molecule at its N-terminus or its C-terminus, see FIG. 2.

It is further possible to produce a fusion molecule comprising more than one molecule in addition to the VHH antibody. For instance, multiple different molecules or multiple copies of a single molecule could be connected or attached to the N-terminus of the VHH antibody, to the C-terminus of the VHH antibody, or at least one molecule is connected or attached to the N-terminus of the VHH antibody and at least one molecule is connected or attached to the C-terminus of the VHH molecule.

In an embodiment, the fusion molecule comprises the at least one other molecule connected or attached to the C-terminus of the VHH antibody.

As mentioned above, the VHH antibody is preferably linked to the at least one other molecule by being genetically fused to the to the at least one other molecule. In such an embodiment, the nucleic acid molecule encodes the VHH antibody and the at least one other molecule as a VHH-containing fusion protein. This means that the nucleic acid molecule comprises a nucleic acid sequence encoding the VHH antibody connected or linked to a nucleic acid sequence encoding the at least one other molecule and where these nucleic acid sequences are under transcriptional control of a same promoter.

The VHH antibody can, however, be linked, connected, attached or conjugated to the at least one molecule in other ways than genetically fused, such as by chemically connecting the VHH antibody to the at least one molecule. For instance, the VHH antibody could be covalently connected to the at least one molecule by a reaction between a maleimide with amines or thiols, often referred to as thiol-maleimide or amine-maleimide click chemistry. In such an embodiment, an additional amino acid residue, such as cysteine, arginine, or lysine, may be added to the N-terminus and/or C-terminus of the VHH antibody to enable such a reaction with maleimide.

It is also possible to enzymatically link the VHH antibody and the at least one molecule, such as by the transglutaminase (TGase) enzyme, which catalyzes the formation of an isopeptide bond between γ-carboxamide groups (—(C═O)NH₂) of glutamine residue side chains and the ε-amino groups (—NH₂) of lysine residue side chains with subsequent release of ammonia (NH₃). In such an embodiment, an additional amino acid residue, such as glutamine or lysine, may be added to the N-terminus and/or C-terminus of the VHH antibody to enable such an enzymatic reaction. It is also possible to prepare site-specific linkage sites through sortase-mediated reactions (Guimaraes et al., Site-specific C-terminal and internal loop labeling of proteins using sortase-mediated reactions, Nate Protocols 8: 1787-1799 (2013)).

In an embodiment, the VHH antibody according to the invention is covalently linked to at least one molecule.

The VHH antibody could be linked, such as fused, directly to the at least one molecule. Alternatively, the VHH antibody is covalently linked to the at least one molecule though a linker. Various such linkers, in particular peptide linkers, could be used according to the embodiments including, but not limited, to Gₙ linkers, wherein n is an integer equal to or larger than 1 and typically equal to or smaller than 10, Sₘ likers, wherein m is an integer equal to or larger than 1 and typically equal to or smaller than 10, A_{q} linkers, wherein q is an integer equal to or larger than 1 and typically equal to or smaller than 10, various GS-linkers or GA-linkers, i.e., combinations of one or more G with one or more S or one or more A, such as (GₙSₘ)ₚ or (SₘGₙ)ₚ or (GₙA_{q})ₚ or (A_{q}Gₙ)ₚ, wherein p is an integer equal to or larger than 1 and typically equal to or smaller than 10. For instance, linkers such as G₄A, G₄S, G₃S, and combinations thereof, such as G₄A-G₄A-G₄S or G₄S-G₃S, could be used. Other commonly used peptide linkers are disclosed in Table 1 of Vishnu Priyanka Reddy Chichil, et al., Linkers in the structural biology of protein-protein interactions, Protein Science 22(″): 153-167 (2013), the linkers listed in Table 1 on pages 156-157 are hereby incorporated by reference as illustrative, but non-limiting, examples of peptide linkers that could be used to covalently interconnect the VHH antibody and the at least one molecule.

The fusion molecule or the VHH antibody of the invention may comprise one or multiple tags, including, but not limited, to affinity purification tags, such as a His tag, a C-tag, a Q-tag, and/or a myc tag. Such a tag may then be present at the N-terminus and/or C-terminus of the fusion molecule or the VHH antibody. Addition of such a tag to enable purification may also be accompanied by a protease site such as, but not limited to, a TEV site for efficient subsequent removal of the purification tag from the VHH antibody or the fusion molecule.

The fusion molecule could also comprise multiple VHH antibodies in addition to at least one molecule as shown in FIG. 2.

In an embodiment, the fusion molecule is a monovalent fusion molecule with regard to the VHH antibody, i.e., preferably only comprises a single VHH antibody in addition to the at last one molecule. There are in vitro and in vivo evidence that the monovalent binding mode facilitates transcellular transport, whereas a bivalent binding mode leads to lysosome sorting (Niewoehner et al., Increased brain penetration and potency of a therapeutic antibody using a monovalent molecular shuttle, Neuron. 81(1): 49-60 (2014)).

The at least one molecule may be any molecule such as a medicament or drug, a diagnostic agent, an imaging agent, a tracer, a half-life extending agent, etc. Examples of such molecules include, without limitation, an antibiotic, antiviral, immunomodulator, antineoplastic, anti-inflammatory, adjuvant, peptides, polypeptides and proteins, such as an enzyme, hormone, neurotrophic factor, neuropeptide, cytokine, apolipoprotein, growth factor, antigen, antibody or part of an antibody, adjuvant, etc., nucleic acids, such as RNA or DNA including e.g., coding genes, inhibitory nucleic acids, such as ribozymes, antisense, interfering nucleic acids, full genomes or portions thereof, plasmids, etc.

In an embodiment, the at least one molecule of the fusion molecule is selected from the group consisting of a therapeutic agent and an imaging agent.

Imaging agent as used herein refers to an agent or molecule that is used during an imaging process to visualize the imaging agent, such as when administered in a patient body, as taken up by cells. Examples of such imaging agents include agents or molecules comprising a radioactive atom, or isotope, such as a radiotracer of a position emission tomography (PET) tracer, such as a ¹⁸F containing PET tracer, a ¹¹C containing PET tracers, ⁶⁴Cu containing PET tracers, or single-photon emission computerized tomography (SPECT) tracers, such as ^{99m}Tc or ¹¹¹In containing SPECT tracers. Other examples of imaging agents include fluorescent probes, luminescent probes, metal complex containing probes, near infrared (NIR) fluorescent probes, etc.

Generally, there is an urgent need for diagnostic tools enabling early detection of diseases or medical conditions, in particular in the brain compartment. Such an early detection would facilitate efficient disease-modifying therapies, especially to evaluate treatment effects in preclinical and clinical trials of new drug candidates.

Specific targeting of disease-causing proteins, including, but not limited to, amyloid beta, tau and alpha-synuclein, or pseudomarkers of disease, such as inflammation, is difficult with classical PET ligands or tracers based on small molecules capable of crossing the BBB. It would generally be preferred to use PET ligands or tracers based on specific binding molecules, such as antibodies or domains of antibodies. However, PET tracers based on monoclonal antibodies (mAbs) generally have too long circulation half-life to be of any use for PET ligands or tracers. This means that the radioactivity originating from radiolabeled mAbs in the blood circulation of the brain will interfere with the specific signal at the brain target site. Therefore, in order to be useful in PET applications, antibody-based PET ligands or tracers should have a fast blood clearance with high brain-to-blood ratio. Smaller antibody domains or fragments (average molecular weight equal to or below 60 kDa), such as VHH antibodies, have a faster systemic elimination as compared to mAbs due to excretion via the kidney. Hence, VHH antibodies, due to their smaller size as compared to mAbs, are suitable as PET ligands or tracers (Syvsnen, et al., A bispecific Tribody PET radioligand for visualization of amyloid-beta protofibrils—a new concept for neuroimaging. Neuroimage 148: 55-63. (2017); Vandesquille, et al., Chemically-defined camelid antibody bioconjugate for the magnetic resonance imaging of Alzheimer's disease. mAbs 9: 1016-1027 (2017)).

In these embodiments, the fusion molecule can be used as diagnostic agent for diagnosis of various diseases or disorders.

For instance, the VHH antibody of the embodiments could be linked to another antibody, antigen-binding fragment or domain thereof, including another VHH antibody, which binds specifically to a disease specific marker. In such a case, the other antibody is preferably labeled to detect its binding to the disease specific marker once delivered, such as to the brain compartment by receptor-mediated transcytosis using the VHH antibody as BBB transporter.

Antigen-binding fragment or domain of an antibody as used herein can be selected from a group consisting of a single chain antibody, a Fv fragment, a scFv fragment, a Fab fragment, a F(ab′)₂ fragment, a Fab′ fragment, a Fd fragment, a single-domain antibody (sdAb), a scFv-Fc fragment, a di-scFv fragment and a group of two or more CDRs.

For therapeutic purposes, the fusion molecule could comprise a half-life extending agent or group, also referred to as a stabilizing agent or group, in addition to the therapeutic agent. Such a half-life extending agent is then included to increase the half-life of the fusion molecule when administered to a patient. Any such half-life extending agent that can be linked to the VHH antibody but with no adverse biological effect could be used including, but not limited to, a Fc fragment of an IgG, serum proteins, such as human serum albumin (HSA), albumin-binding protein scaffolds, an IgG, various polyethylene glycol (PEG) molecules, or unstructured polypeptides, such as XTEN, or PASylation as a substitute for PEGylation.

The present invention also relates to a pharmaceutical composition comprising a fusion molecule as defined above, wherein the at least one molecule is a therapeutic agent. The pharmaceutical composition also comprises a pharmaceutically acceptable vehicle or excipient.

The pharmaceutically acceptable vehicle could be any pharmaceutically acceptable vehicle or carrier that is compatible with the other constituent(s) of the pharmaceutical composition. The pharmaceutically acceptable vehicle can be selected from the vehicles traditionally used according to each mode of administration. The pharmaceutical composition could be a solid pharmaceutical composition, such as a tablet, pill, powder, or granules, a semi solid pharmaceutical composition, such as a suppositories; or a liquid pharmaceutical composition, such as soft capsule or injection solution.

Pharmaceutically acceptable vehicles or excipients include, but are not limited to, diluents, such as lactose, dextrose, sucrose, mannitol, sorbitol, cellulose, and glycine; lubricants, such as silica, talc, stearic acid including salts thereof, and polyethylene glycol; binders, such as magnesium and aluminum silicate, starch paste, gelatin, tragacanth, methylcellulose, sodium carboxymethyl cellulose, and polyvinylpyrrolidone; disintegrants, such as starch, agar, alginic acid, and sodium alginate; absorbents; dyes; flavoring agents; sweeteners; polypropylene glycol; liquid vehicles, such as water, physiological saline solution, aqueous dextrose, glycerol, ethanol, and oil.

The embodiments also relate to a fusion molecule according to above for use as a medicament, wherein the at least one molecule is a therapeutic molecule.

In an embodiment, the therapeutic agent is capable of treating a CNS disease or disorder.

In a particular embodiment, the fusion molecule according to above is for use in treatment of a CNS disease or disorder. In such an embodiment, the at least one molecule is therapeutic agent capable of treating the CNS disease or disorder.

A further embodiment is directed towards a method of treating a CNS disease or disorder in a patient. The method comprises administering an effective amount of a fusion molecule according to above or a pharmaceutical composition according to above to the patient. In such an embodiment, the at least one molecule is therapeutic agent capable of treating the CNS disease or disorder.

Illustrative, but non-limiting, examples of CNS disease or disorders include Alzheimer's disease (AD), Bell's palsy, cerebral palsy, epilepsy, motor neuron diseases (MND), such as amyotrophic lateral sclerosis (ALS), progressive bulbar palsy (PBP), pseudobulbar palsy, progressive muscular atrophy (PMA), primary lateral sclerosis (PLS), spinal muscular atrophy (SMA) and monomelic amyotrophy (MMA), multiple sclerosis (MS), neurofibromatosis, Parkinson's disease (PD), lysosomal storage diseases, neuronopathic lysosomal storage diseases, ischemic stroke, intracerebral hemorrhage, traumatic brain injury (TBI), vascular dementia, frontotemporal dementia, amyloidosis, tauopathy, Creutzfeldt-Jakob disease, neuroinflammation and neuropathic pain.

Malignant cells often overexpress TfR1 and this increased expression can be associated with poor prognosis in different types of cancer (Candelaria et al., Antibodies Targeting the Transferrin Receptor 1 (TfR1) as Direct Anti-cancer Agents. Front. Immunol. 12: 607692 (2021)). TfR1 is overexpressed on many different types of cancer cells, often at levels several-fold higher than normal cells. In fact, TfR1 has been identified as a universal cancer marker. Increased expression of TfR1 correlates with advanced stage and/or poorer prognosis in a number of cancers, including solid cancers, such as esophageal squamous cell carcinoma, breast cancer, ovarian cancer, lung cancer, cervical cancer, bladder cancer, osteosarcoma, pancreatic cancers, cholangiocarcinoma, renal cell carcinoma, hepatocellular carcinoma, adrenal cortical carcinoma, glioblastoma multiforme (GBM) and cancers of the nervous system, including brain metastases of peripheral cancers, as well as hematopoietic malignancies, such as acute lymphoblastic leukemia (ALL), chronic lymphocytic leukemia (CLL), and non-Hodgkin lymphoma (NHL) (Candelaria et al., Antibodies Targeting the Transferrin Receptor 1 (TfR1) as Direct Anti-cancer Agents. Front. Immunol. 12: 607692 (2021); Ramalho et al., Transferrin Receptor-Targeted Nanocarriers: Overcoming Barriers to Treat Glioblastoma. Pharmaceutics 14(2): 279 (2022)).

The elevated levels of TfR1 expression on malignant cells, together with its extracellular accessibility, ability to internalize, and central role in cancer cell pathology make this receptor an attractive target for antibody-mediated therapy. The TfR1 can be targeted by VHH antibodies of the invention for cancer therapy through the use of VHH antibodies conjugated to anti-cancer agents, immunotherapy agents and/or adjunctive therapy agents that are internalized by receptor-mediated endocytosis.

In another embodiment, the therapeutic agent is capable of treating cancer.

In a particular embodiment, the fusion molecule according to above is for use in treatment of cancer. In such an embodiment, the at least one molecule is therapeutic agent capable of treating cancer.

A further embodiment is directed towards a method of treating cancer in a patient. The method comprises administering an effective amount of a fusion molecule according to above or a pharmaceutical composition according to above to the patient. In such an embodiment, the at least one molecule is therapeutic agent capable of treating cancer.

The hTfR1 has also been a target to deliver therapeutic agents, including oligonucleotides, to muscle (Desjardins et al., Enhanced exon skipping and prolonged dystrophin restoration achieved by TfR1-targeted delivery of antisense oligonucleotide using FORCE™ conjugation in mdx mice, Nucleic Acids Research gkac641, 2022; Sugo et al., Development of antibody-siRNA conjugate targeted to cardiac and skeletal muscles, J Control Release 237: 1-13 (2016)). Accordingly, the fusion molecule of the invention can be used to treat various muscular diseases and in particular muscular dystrophy.

The fusion molecule could also be used to treat muscular dystrophy, such as Duchenne muscular dystrophy (DMD), Becker muscular dystrophy (BMD), mytonic muscular dystrophy (DM), limb-girdle muscular dystrophy (LGMD), facioscapulohumeral muscular dystrophy (FSHD), congenital muscular dystrophy (CMD), distal muscular dystrophy (DD), oculopharyngeal muscular dystrophy (OPMD), or Emery-Dreifuss muscular dystrophy (EDMD), in particular DMD, FSHD or DM.

In a further embodiment, the therapeutic agent is capable of muscular dystrophy.

In a particular embodiment, the fusion molecule according to above is for use in treatment of muscular dystrophy. In such an embodiment, the at least one molecule is therapeutic agent capable of treating muscular dystrophy.

A further embodiment is directed towards a method of treating muscular dystrophy in a patient. The method comprises administering an effective amount of a fusion molecule according to above or a pharmaceutical composition according to above to the patient. In such an embodiment, the at least one molecule is therapeutic agent capable of treating muscular dystrophy.

As used herein, effective amount indicates an amount effective, at dosages and for periods of time necessary to achieve a desired result. Effective amounts may vary according to factors, such as the disease state, age, sex, weight of the patient. Treating or treatment as used herein and is well understood in the art, means an approach for obtaining beneficial or desired results, including clinical results. Beneficial or desired clinical results could include, for instance, alleviation or amelioration of one or more symptoms or conditions, diminishment of extent of disease, stabilized state of disease, i.e., prevent worsening, preventing spread of disease, delay or slowing of disease progression, amelioration or palliation of the disease state, diminishment of the reoccurrence of disease, and remission. Treating or treatment may also prolong survival as compared to expected survival if not receiving any treatment.

Preventing or prophylaxis as used herein and is well understood in the art, means an approach in which a risk of developing a disease or condition is reduced or prevented, including prolonging or delaying disease development. For instance, a patient predisposed to develop a disease, such as due to genetic or hereditary predisposition, could benefit for administration of the fusion molecule or the pharmaceutical composition to prevent, reduce the risk of, delaying and/or slowing development of the disease.

The patient is preferably a human patient. The embodiments may, however, also be applied in veterinary applications, i.e., non-human patients, such as non-human mammals including, for instance, primates, monkeys, apes, cattle, sheep, pigs, goats, horses, cats, dogs, mice, rats and guinea pigs.

The fusion molecule or the pharmaceutical composition according to the embodiments may be administered to the patient according to various routes including, for instance, parenteral route, such as injection by subcutaneous, intravenous, intraperitoneal or intramuscular route; oral route; rectal route; topical route; intranasal route; perlingual route; or intraocular route. In a particular embodiment, the administration route is a subcutaneous or intravenous administration.

### EXAMPLES

### Example I—Production of Proteins for Llama Immunization and Assays

The extracellular domains of hTfR1 (amino acids R121-F760, SEQ ID NO: 30), mTfR1 (S122-F763, SEQ ID NO: 31) and cTfR1 (R121-F760, SEQ ID NO: 32) were cloned into a pCDNA3.4-TOPO plasmid with the 5′ cloning sites: HindIII, KpnI, NheI and 3′ cloning sites: XhoI, Pac, using stop codons: TGA, TGA and a Kozak sequence (generated with Geneart tool (Thermo Fischer Scientific)), optimized for expression in Cricetulus griseus. An N-terminal signal peptide (MDWLRNLLFLMAAAQSINA, SEQ ID NO: 33) followed by a single amino acid linker “A”, a Histidine tag (6×His, SEQ ID NO: 38) and an SG-linker (hTfR1, cTfR1) or a G-linker (mTfR1) were used for expression. Plasmids were ordered from Geneart (Thermo Fischer Scientific, Germany). Transient transfection of Chinese hamster ovarian (CHO) cells was performed according to standard procedures. Cultures were harvested on Day 6. Supernatants were filtered (0.45 μm syringe filter) before purification with immobilized metal ion chelate (IMAC) using a HisTrapExcel 5 mL column (Cytiva). Buffers used were as follows: equilibrium/wash (50 mM phosphate, 500 mM NaCl, pH 8), primary wash buffer (50 mM phosphate, 500 mM NaCl, 20 mM Imidazole, pH 8) and elution buffer (50 mM phosphate, 500 mM, NaCl 500 mM, Imidazole, pH 8). Eluted fractions were concentrated with an Amicon Ultra 10000 MWCO spin concentrator. Proteins were dialyzed overnight in PBS with 10% glycerol (Medicago, tablet, glycerol diluted from 80% stock solution) at 4° C. (cassette slide-a-lyzer 3 ml), 10 kDa cutoff (Pierce). PBS was set to pH 8. The protein concentrations were measured using absorbance at 280 nm.

### Example II—Generation of VHH Library that Bind the Human TfR1 and Selection

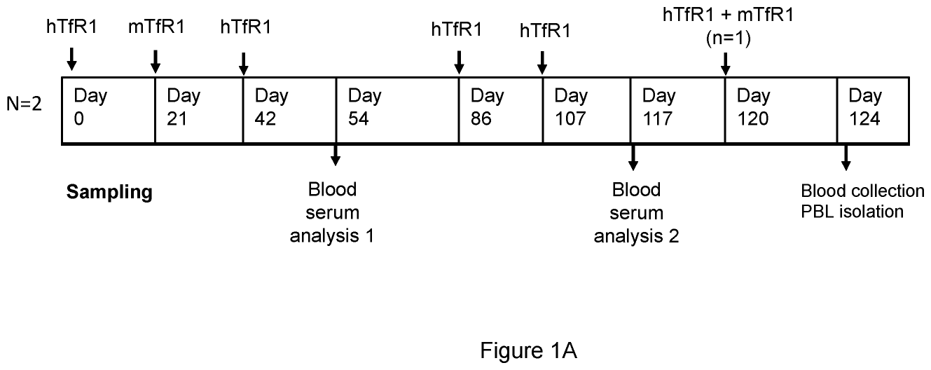

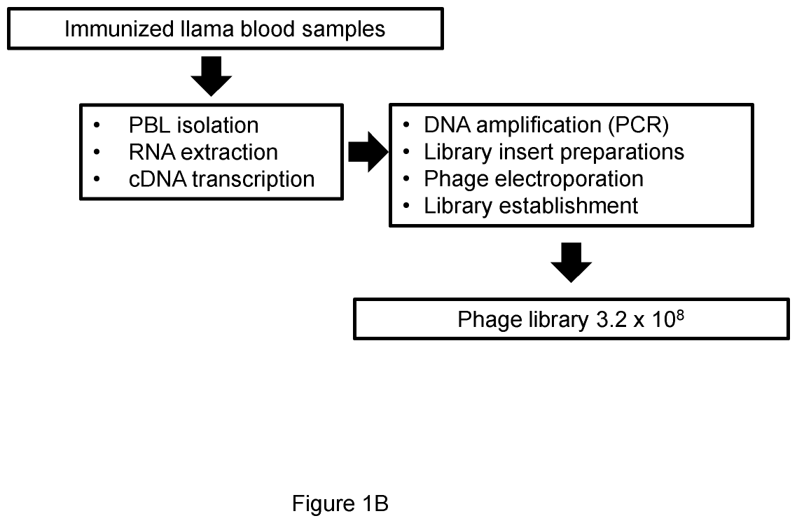

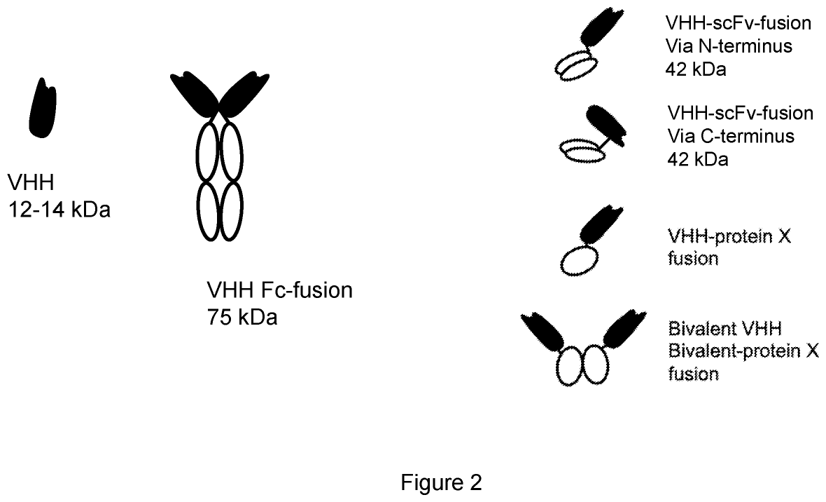

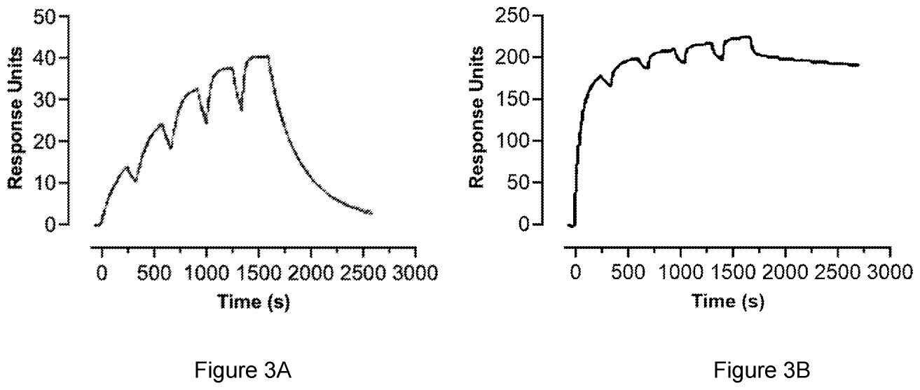

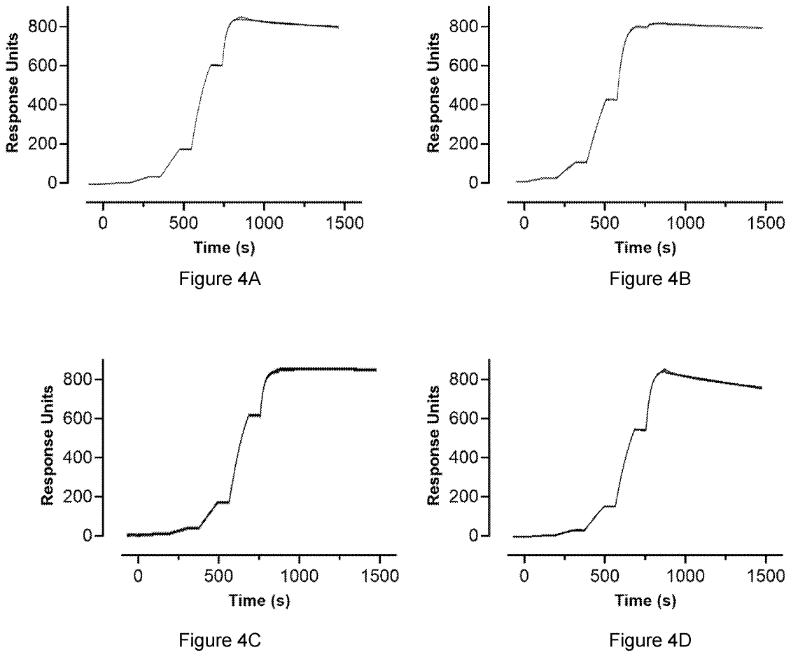

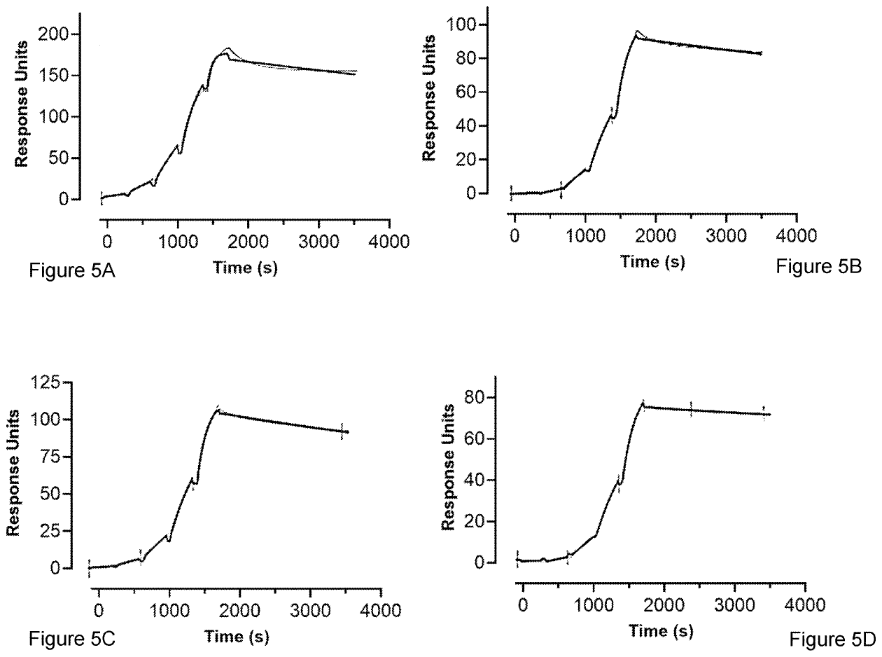

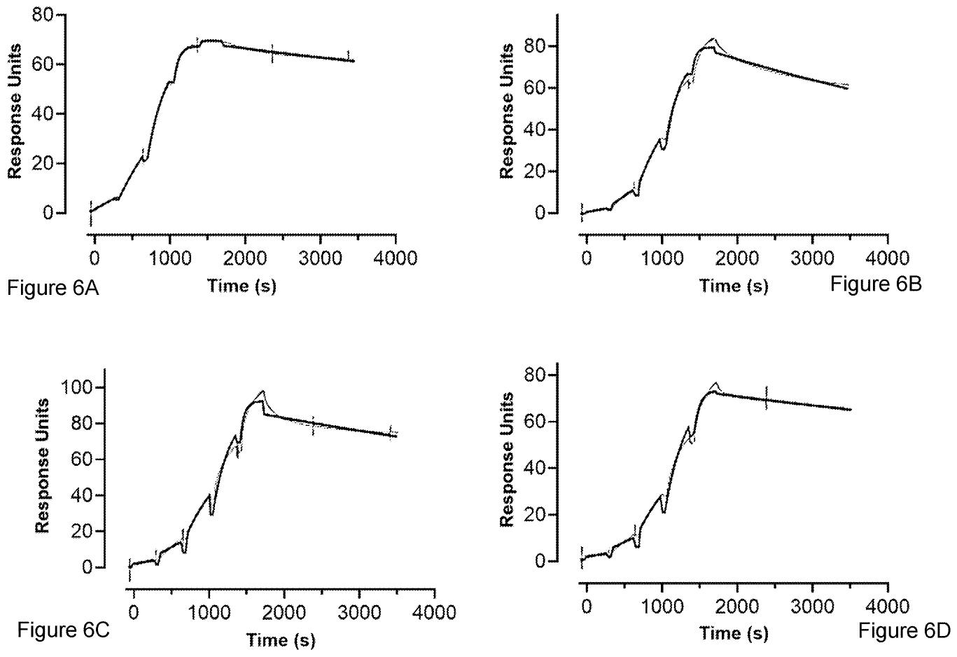

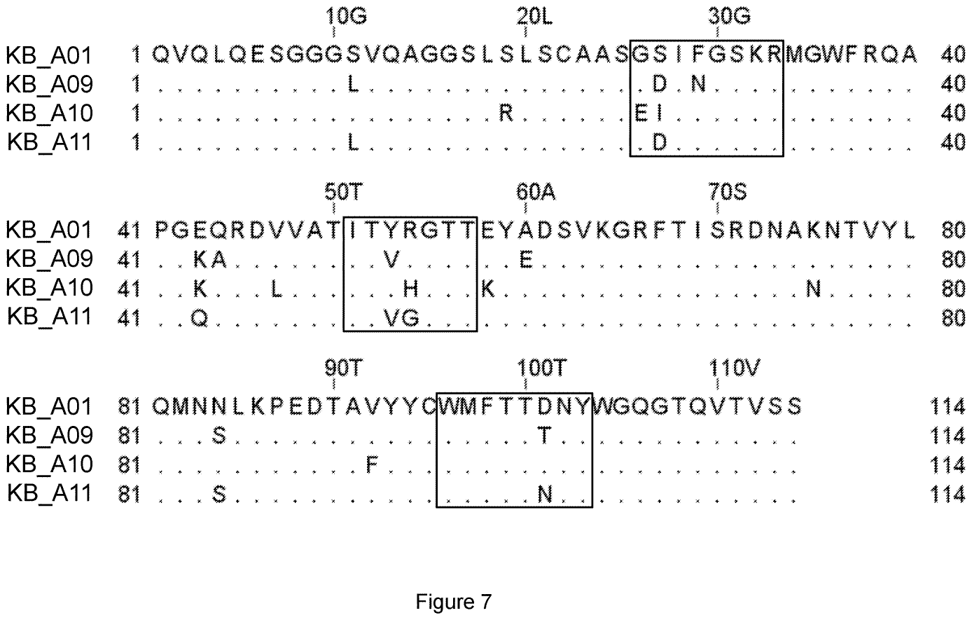

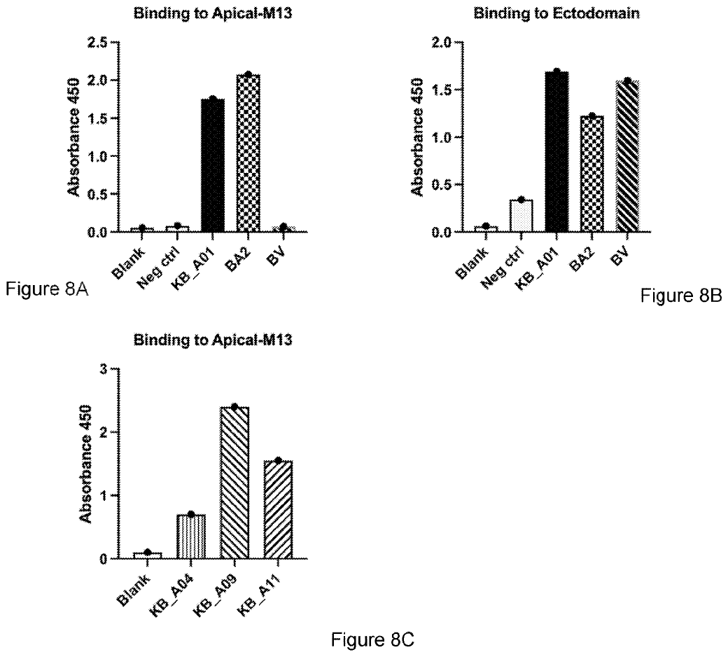

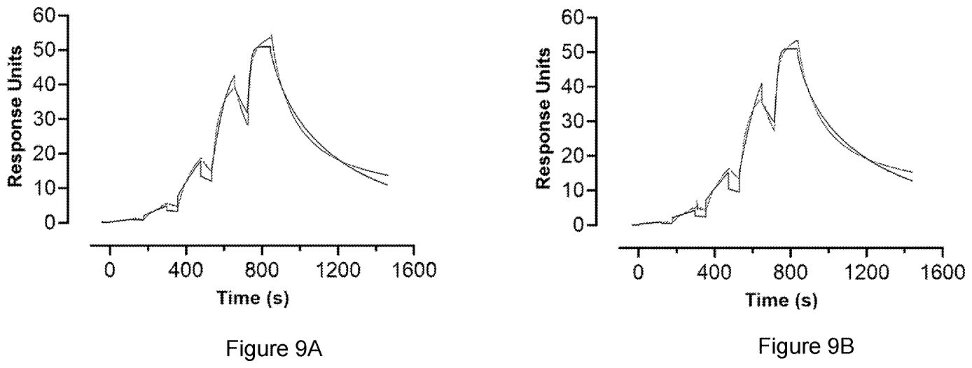

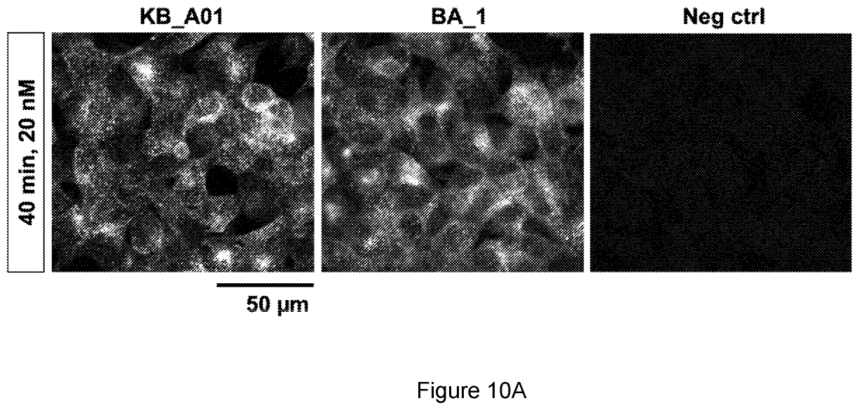

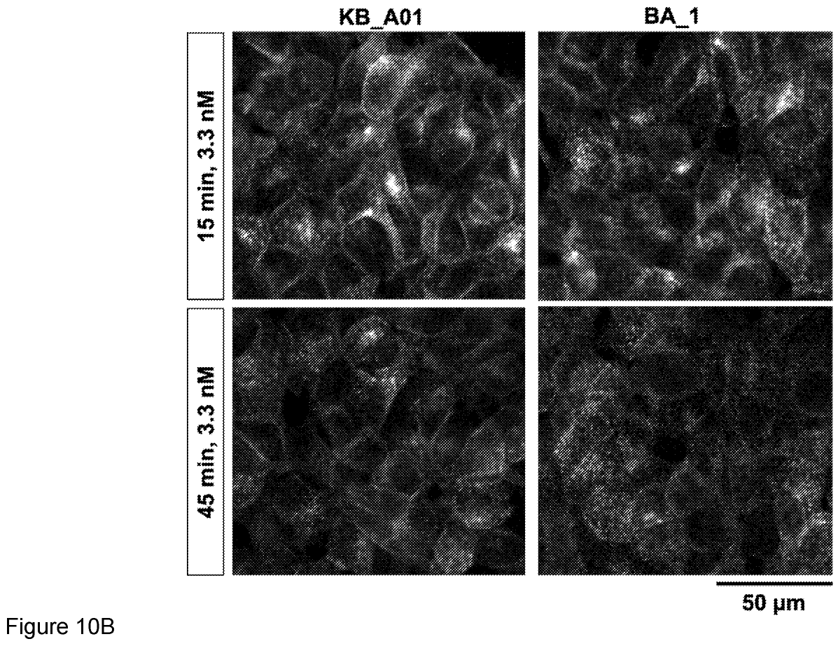

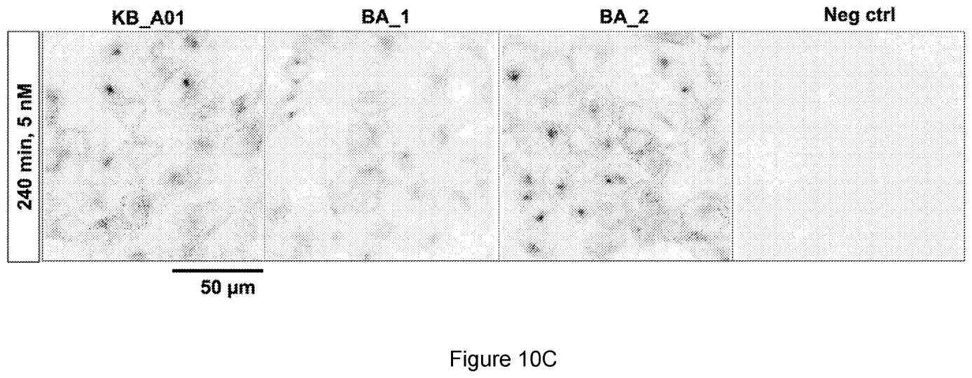

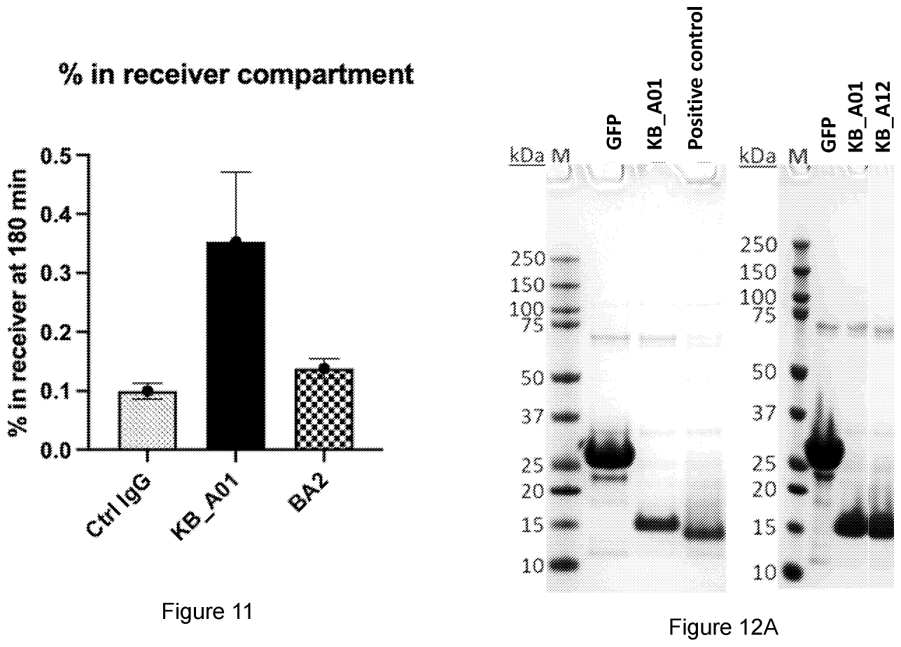

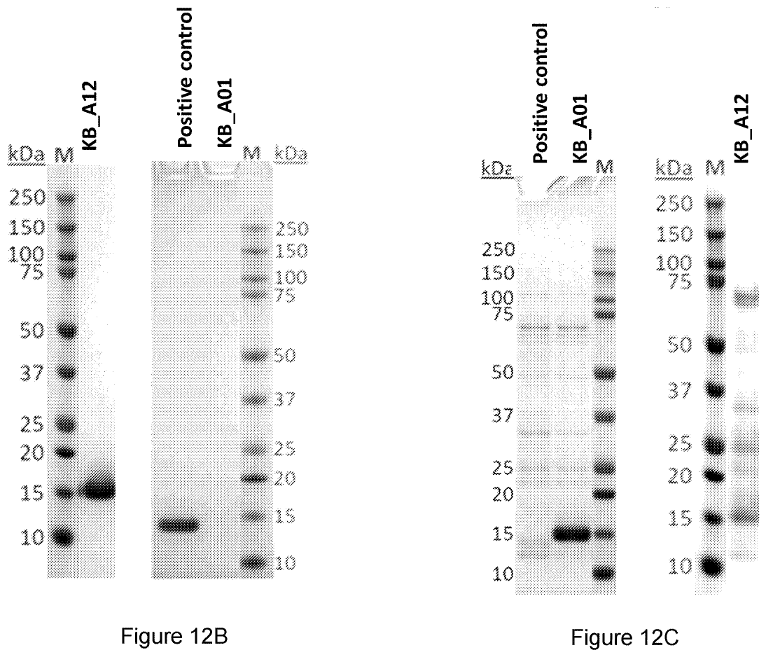

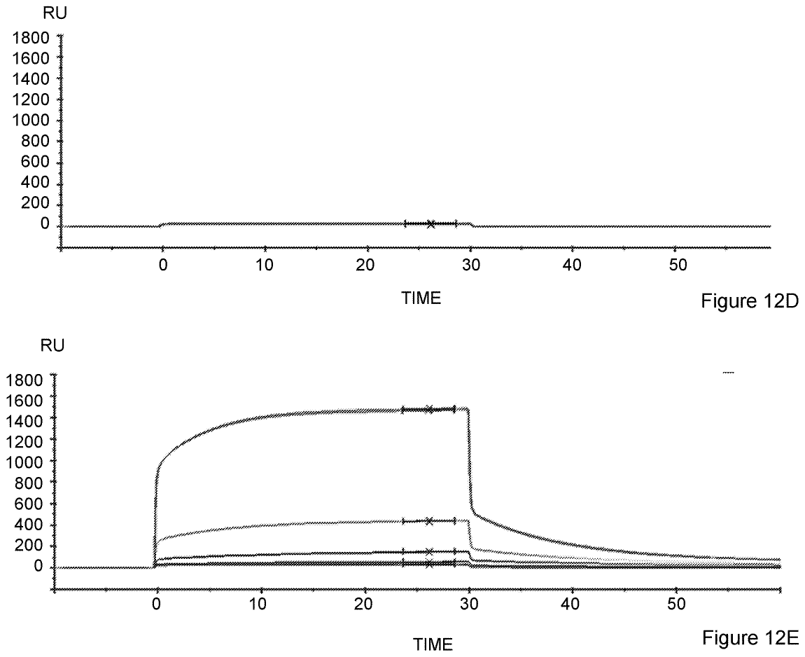

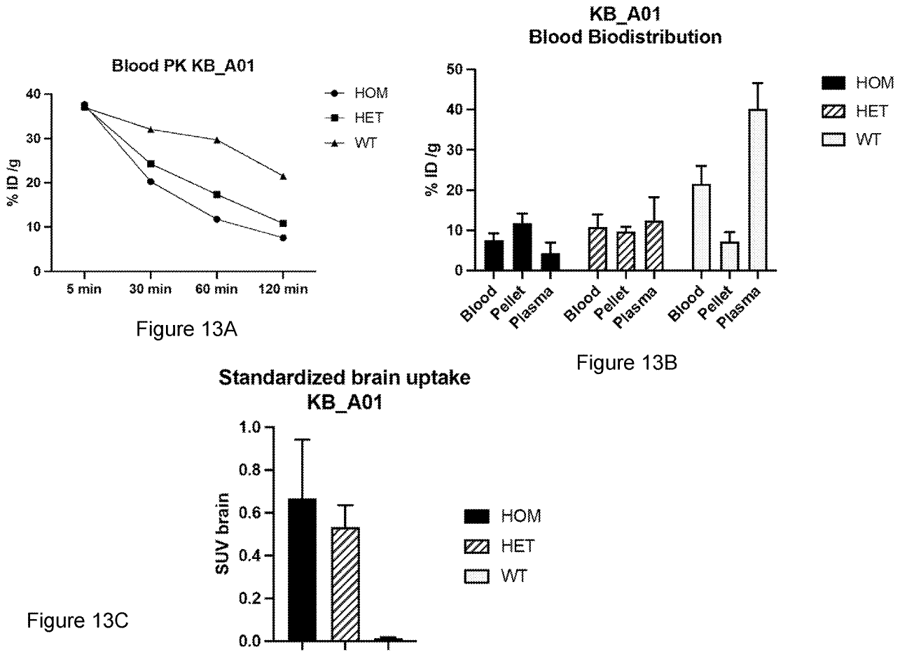

Immunization for antibody generation in llama (Lama glama) animals (N=2) was performed according to the ModiPhage™ method at Modiquest Research BV (the Netherlands). Primary immunization was performed in two llama animals with an adaptive immunization protocol based on the monitoring of specific immune response to the immunogens hTfR1 and mTfR1 (produced as described in Example I). Immunization was made with a dose of 500 μg protein, intramuscular (i.m.) as shown in FIG. 1 on Day 1 (hTfR1), Day 21 (mTfR1) and Day 42 (hTfR1). On Day 54, blood withdrawal was performed to study immune response with ELISA. Serum samples were serially diluted in 3-fold steps (from 1:100 to 1:72900) and screening towards plate-immobilized hTfR1, mTfR1 and control protein bovine serum albumin (BSA), all at 1 μg/ml. Specific binding of anti-llama IgG1, IgG2/IgG3 antibodies were detected with goat anti-rabbit-horse radish peroxidase (HRP) and goat anti-mouse-HRP respectively, detected with 3,3′,5,5′-tetramethylbenzidine (TMB) substrate at absorbance 480 nm in a spectrophotometric plate reader. Two additional doses with 500 μg hTfR1 were delivered on Day 86 and Day 107. On Day 117, blood was drawn and ELISA was performed as described above. One animal was selected for final harvest, also received an additional boost dose with a 1:1 mixture of hTfR1 and mTfR1 (250 μg+250 μg) on Day 120. Four days later (Day 124) peripheral blood was withdrawn for PBL isolation by density gradient centrifugation using Ficoll-Paque™ PLUS ˜1.5×10⁹. RNA extraction was made from −0.7×10⁹ PBLs using TRIzol (Thermo Fischer Scientific). Reverse transcription to cDNA was performed by oligo(dT)-primed reverse transcription of 400 μg freshly isolated RNA.

All subsequent steps to construct the phage display library of VHHs was done as described in Pardon et al., A general protocol for the generation of Nanobodies for structural biology. Nat Protoc 9: 674-693 (2014), see FIG. 1B. In more detail, cDNA from above was used as a template for a PCR reaction to amplify the IgG2/3 repertoire. A first PCR reaction was performed to amplify all antibodies (IgG1 and IgG2/3) followed by a second nested PCR reaction to amplify and isolate the VHH repertoire, including overhang sites for subcloning into the phagemid pADL10b using primers CALL001 (SEQ ID NO: 34) and CALL002 (SEQ ID NO: 35). An amount of 3 μg of cDNA template was used with the High Fidelity® kit from ThermoFischer Scientific: 10 μl 5× High Fidelity buffer, 1 μl dNTPs (dinucleotide triphosphates; 10 mM of each, 0.2 mM final concentration), 25 pmol per primer (0.5 μM final primer concentration), 0.5 μl Phusion® polymerase (1 U/reaction), ddH₂O (double-distilled water) to 50 μl, 1-2 μl template with cycling program: 98° C. 5 min, 98° C. 30 s (30×), 72° C. 30 s (30×), 72° C. 10 min, 4° C. storage. A total of 36 wells were pooled and then purified using Genejet enzymatic cleanup (Thermo Fischer, cat #K0832) with PCR dimer removal. The mixture was purified using four columns and eluted in 4×20 μl elution buffer. The eluate was loaded on a 1% agarose gel with SYBRSafe DNA GelStain (SigmaAldrich) safe for gel extraction and the extracted product was used as template in a subsequent PCR reaction. Next, nested PCR reactions were used for amplification including pADL10 cloning sites using the following oligonucleotides VHH-R-Sfil (SEQ ID NO: 36) and VHH-F-Sfil (SEQ ID NO: 37). Two PCR mixtures were prepared with 0.5 μM per primer (VHH-F-Sfil and VHH-R-Sfil) and 0.1 μM per primer, respectively and 1 μl template Thermocycling was done as follows: 98° C. 5 min, 98° C. 30 s (20×), 72° C. 1 min (20×), 72° C. 10 min, 4° C. storage. About 50 ng of amplified and gel extracted heavy chain antibody was used as template in each well. All wells were pooled after amplification and purified using Genejet PCR cleanup according to the manufacturers protocol and eluted in elution buffer (10 mM Tris-HCl pH 8.5). PCR product concentration was measured with Nanodrop (result: 160 ng/μl). The products were digested with Sfil restriction enzyme (Thermo Fischer Scientific). DNA fragments at 400 bp were extracted on a 2% agarose gel using Genejet gel extraction.

The pADL10b vector was digested according to manufacturer's instructions using Sfil restriction enzyme (Thermo Fischer Scientific) overnight at 37° C. Digested vectors were purified on a 1% agarose gel and visualized using Sybr Safe (Thermo Fischer Scientific). The procedure was repeated to a final concentration of 154.6 ng/μl.

After PCR amplification, the library was digested (using Sfil) for 2 h at 50° C. Enzymes were removed using a Genejet PCR cleanup column as above. The resulting material was eluted in 20 μl elution buffer (10 mM Tris-HCl pH 8.5). DNA concentration was measured to 73 ng/μl. The mixture was ligated again. First, the material was heated at 50° C. and slowly cooled back to room temperature (RT, 20-25° C.) followed by incubation overnight at 16° C. with shaking. Cleanup was performed as above and eluted in 10 mM Tris-HCl pH 8.5. A total of 1600 ng DNA was used. 1 or 2 μl of ligation mixture was used to electroporate 10 μl TG1 Escherichia coli in 25 μl total reaction volumes (5.48 ms 1.92 kV and 5.48 ms 1.92 kV, respectively). Cells were used immediately after retrieval from the −70° C. freezer and thawing on ice. Transformants were distributed on large agar plates (20 cm) and allowed to grow under standard conditions until colonies present and ready to harvest.

Cells were harvested by adding Luria Broth (LB) medium. Colonies were counted, the library size estimated to 3.2×10⁸. Cultures were prepared of the final library pool after overnight growth on agar plates. The cultures were shaken at 200 rotations per minutes (rpm) at 37° C. for 2 h. Hyperphage was added with a multiplicity of infection (MOI) of 2, for 30 min at 37° C. without shaking. The media was removed by centrifugation at 4500 g for 5 min in Falcon tubes. The cells were then re-suspended in an equal volume of Terrific Broth (TB) medium supplemented with 100 μg/ml ampicillin, 50 μg/ml kanamycin and 1 mM isopropyl β-d-1-tiogalaktopyranosid (IPTG). Cultures were shaken overnight at 30° C. at 200 rpm.

5 mL cultures were prepared in LB from overnight cultures of selected clones in 15 mL falcon tubes. supplemented with 100 μg/ml ampicillin, glucose was omitted. Overnight cultures were diluted 1:100 in 5 ml LB and grown for 2 h at 150 rpm until the cultures were turbid. 1 μl hyperphage helper phage (Hyperphage, titer: 1.4×10¹² cfu/ml, Progen) was added to each culture. The helper phage was allowed to infect for 30 min at 37° C. Phage-containing LB was discarded by centrifugation at 4500 g. Bacterial pellets were re-suspended in 5 ml TB medium supplemented with 100 μg/ml ampicillin, 50 μg/ml kanamycin and 1 mM IPTG. The cultures were shaken at 200 rpm and 30° C. for over-night. Cultures were centrifuged at 4500 g for 4 min to remove the bacteria, adding 0.25×V of 20% PEG8000/2.5 M NaCl was added and incubated on ice for 2 h in centrifuge tubes. The phage/bacteria were pelleted at 15000 g for 10 min and re-suspended in 0.5 ml PBS. The phage-containing supernatants were transferred to separate microcentrifuge tubes pre-blocked with PBS/casein.

Panning with Dynabeads® with Biotinylated TfR1

Biotinylation of hTfR1, cTfR1 and mTfR1 was performed according to standard procedures with a biotinylation kit EZ-link Sulfo NHS PEG4 (Thermo Fischer Scientific) using 1 mg target protein with a mixture of 2 mM biotin at a 1:1 molar ratio at pH 8. Free biotin was removed by dialysis in a Pierce 3 mL 10 kDa MWCO (molecular weight cut-off) dialysis unit, at 4° C. with 3×1 h with 1 L PBS buffer at pH 8. The protein was concentrated to 850 μl with Amicon spin concentration 10 kDa MWCO. The phage supernatants were pre-blocked with PBS/casein and mixed with 100 nM of biotinylated hTfR1, cTfR1 or mTfR1, and incubated for 1 h. Bead blocking was done using 50 μl Streptavidin Dynabeads® (M280, Invitrogen, Thermo Fischer Scientific) with hTfR1 (1.1 mg/mL) and mTfR1 (1 mg/mL) and washed with PBS with 0.15% Tween® 20 (PBST). The beads were re-suspended in 1 ml ELISA blocker (PBS/casein ELISA blocker (Thermo Fischer Scientific)) and incubated for 30 min in a rotamixer at RT. Blocker was replaced by the phage+target mixture, incubated for 30 min at RT with gentle rotation to capture the biotinylated TfR1 on the beads. Beads were washed 5 times with PBS. Both mTfR1 and hTfR1 captures were eluted in 400 μl PBS with 0.25 mg/ml trypsin.

Phages were trypsinated for 30 min at RT before titration and rescue. A TG1 Escherichia coli culture was started by inoculating a 10 ml culture in 10 ml of LB medium with a single colony of TG1 grown on a M9 minimal agar plate. The culture was incubated at 37° C. at 200 rpm until the OD₆₀₀ had reached log phase (around 4 h, OD₆₀₀ at around 0.6). The culture was kept on ice, dilutions (1-10 times) prepared and mixed with 100 μl TG1 and incubated for 5 min before plating the mixtures on LB Lennox agar plates (Merck-Sigma Aldrich) supplemented with 2% glucose and 100 μg/ml ampicillin. The input was first trypsinated (1 mg/ml trypsin) by mixing 75 μl of the phage stock with 25 μl and incubated at RT for 30 min before dilution and titration. The entire remaining phage outputs were mixed with 2 ml TG1 E. coli each and incubated at RT without shaking for 5 min. The mixtures were plated on two large LB Lennox agar plates each and allowed to dry completely before incubating at 37° C. in plastic bags.

The procedure for selection with capture Dynabeads® (and subsequent rescue) was repeated in three subsequent rounds with either hTfR1, cTfR1 or mTfR1 at 100 nM, 25 nM, 10 nM and 5 nM (the last two in the presence of 1 μM human transferrin (recombinant made in rice, Sigma Aldrich, human Transferrin)), according to Table 1 below. After the 4^{th} round of selection, 25 colonies were randomly picked from agar plates representing each track and elution method (trypsin or citrate/phosphate buffer of pH of 5.5 with 0.15% Tween® 20), sent for sequencing with primer pADL10fwd at Karolinska Institute (Sweden) genetic analysis core facility.

TABLE 1
Biopanning scheme for selection

| Panning | | | | | Concentration | | Transferrin |
|---|---|---|---|---|---|---|---|
| round | 1 | 2 | 3 | 4 | TfR1 | | (Tf) |
| Selection 1 | hTfR1 | hTfR1 | mTfR1 | mTfR1 | 100 | nM | No |
| Selection 2 | hTfR1 | mTfR1 | mTfR1 | hTfR1 | 25 | nM | No |
| Selection 3 | cTfR1 | cTfR1 | mTfR1 | mTfR1 | 10 | nM | 1 μM |
| Selection 4 | hTfR1 | mTfR1 | mTfR1 | hTfR1 | 5 | nM | 1 μM |

| | | | | | | | | | | |
|---|---|---|---|---|---|---|---|---|---|---|
| Elution | pH | tryp | pH | tryp | pH | tryp | pH | tryp | — | — |
| method | | | | | | | | | | |

| | | | | | | | | | |
|---|---|---|---|---|---|---|---|---|---|
| Clones | 25 | 25 | 25 | 25 | 25 | 25 | 25 | 25 | Total picked |
| (n =) | | | | | | | | | clones N = 200 |

### Example III—Binding with ELISA to Select VHHs Binding the hTfR1 and cTfR1

Selected phage samples were diluted 1:10 in block buffer casein ELISA blocker (ThermoFischer Scientific), 20% v/v PEG8000 in 2.5 M NaCl before the ELISA. The targets (hTfR1, cTfR1, mTfR1) were coated at 1 μg/ml in PBS pH 7.4 overnight at 4° C. in 96-well Greiner plates (non-treated), 100 μl. The coating buffer was discarded and replaced by 100 μl block solution. The plate was covered and incubated at RT for 2 h, 900 rpm. Anti-human TfR1 antibody BA3 (MEM189, Sigma, 1 mg/ml) and anti-mTfR1 (81D3, Novus biologicals, 1 mg/ml) were diluted in block buffer to 100 nM starting concentration and diluted 1:2 in series by transferring 100 μl to 100 μl block buffer. Plates were incubated for 1 h at RT, 900 rpm, Wells were washed 3×300 μl in PBST (PBS with 0.1% Tween® 20) using the 50TS microplate washer (Biotek). Anti-M13 antibody (Anti-PVIII GE Healthcare) diluted 1:4000 in block buffer, 100 μl was added to target-coated wells followed by incubation for 1 h at RT, 900 rpm. Washing with PBST was performed. The secondary anti-mouse antibody (Sigma) 1:4000 in block buffer, incubated 1 h at RT, 900 rpm. Washes in PBST was followed by development with TMB substrate. The reaction was stopped with addition of 2 M H₂SO₄ to each well, readings for absorbance at 450 nm was measured using a SpectraMax 3000 (Molecular Devices). One clone (KB_A01) used in the following examples was selected based on cross-reactive binding in the ELISA to both cTfR1 and hTfR1 with no reactivity to mTfR1. The criteria set for selection was reactivity to both human and cynomolgus receptor>10 times above blanks (negative controls). The selection criteria in this workflow was also based on non-competition with Tf for the Tf binding site (see Example II), which is a non-preferred binding site for brain transport of therapeutics.

### Example IV—Production of Free VHHs or Fusion Variants

Selected VHH clone (KB_A01, SEQ ID NO: 23) was produced as single VHH units (12-14 kDa), see FIG. 2, with a C-terminal 6×His tag (SEQ ID NO: 38) followed by a “C-tag” (C-terminal amino acids EPEA, SEQ ID NO: 39) ordered and codon-optimized from Genscript Inc. using expression Vector pET-22b(+). Protein was obtained from the periplasmic space in Escherichia coli and purified using one-step purification on a Ni-column.

Human Fc-region was genetically fused to VHHs with a GG spacer creating a bivalent TfR1-binding functional entity, see FIG. 2. In more detail, a VHH Fc fusion protein (SEQ ID NO: 41) between human IgG1 Fc (SEQ ID NO: 40) and KB_A01 (SEQ ID NO: 23) with signal peptide from murine IgG kappa light chain (SEQ ID NO: 61) was cloned into a vector pcDNA3.4 backbone with codon-optimized sequences (performed at GeneArt, Thermo Fischer Scientific) and transiently expressed in Expi293™ cells (Thermo Fischer Scientific), 200 mL cultures grown in standard conditions and harvested 6 days after transfection. Purification of culture medium was done on a MabSelect Sure™, polished in a HiLoad Superdex 200 and yielded>99% pure fractions of dimers VHH-Fc fusions, as verified by analytical size-exclusion chromatography (SEC). The same process was done to produce a fusion protein for a benchmark VHH (SEQ ID NO: 60) as disclosed in WO 2020/144233 and human IgG1 Fc. This benchmark VHH-containing fusion protein is referred to as BV herein.

Following a bioinformatics assessment, yet other VHH-Fc fusions were made between the human IgG1 Fc and KB_A02 (SEQ ID NO: 42), KB_A03 (SEQ ID NO: 24), KB_A04 (SEQ ID NO: 25), KB_A05 (SEQ ID NO: 43), KB_A06 (SEQ ID NO: 44), KB_A07 (SEQ ID NO: 26), KB_A08 (SEQ ID NO: 45), KB_A09 (SEQ ID NO: 27), KB_A10 (SEQ ID NO: 28) and KB_A11 (SEQ ID NO: 29). These other VHH-Fc fusions were produced in Turbo-CHO™ High Performance platform systems following sequence optimization and cloning into proprietary plasmids at GenScript Biotech Corporation in a volume of 4 mL, based on the same human IgG1 Fc and GG linker sequences as above and a signal peptide (SEQ ID NO: 56). Here, final samples were also harvested on Day 6 after transfection, purified on protein A-based systems at Genscript yielding 95-99% purity as verified by either SDS-PAGE or analytical size-exclusion chromatography (SEC).

Functional fusion proteins were produced in the form of fusions with VHH and a single-chain variable domain (scFv). Such a fusion protein has monomeric TfR1 binding but potential for bispecific binding, due to the presence of the scFv domain, which is able to bind a different target than TfR1 or a different epitope on the same target. The amyloid beta-binding antibody 3D6 was chosen as a functional and representative scFv unit. The scFv of the 3D6 antibody was made up by VH (SEQ ID NO: 46) and VL (SEQ ID NO: 47) fused to either the N-terminus or the C-terminus of KB_A01 joined by a 3×(G₄S) linker (SEQ ID NO: 48) resulting in KB_A01-scFv (SEQ ID NO: 49) or scFv-KB_A01 (SEQ ID NO: 50), see FIG. 2. Genetic constructs were optimized for expression in CHO, performed by GenScript Biotech Corporation and expressed in transiently transfected into TurboCHO™ High Performance platform cells.

Benchmark antibody 128.1 (BA1) was expressed as a full length human IgG1 (SEQ ID NO: 51) in transiently transfected FreeStyle™_293 using vector pcDNA3.4 and harvested from medium, purified in one-step purification on protein A resin. This benchmark antibody BA1 binds to a region of the extracellular portion of hTfR1 and has the ability to cross the BBB (WO 93/10819; Friden P M, et al., Characterization, receptor mapping and blood-brain barrier transcytosis of antibodies to the human transferrin receptor. J Pharmacol Exp Ther 278: 1491-1498 (1996)).

Benchmark antibody JCR-IgG (BA2) a clinically validated CNS targeting TfR1 binding antibody was designed based on information in U.S. Pat. No. 9,994,641 as an IgG1 antibody reactive to hTfR1 through its Fab′2 (SEQ ID NO: 51). Heavy chain (HC) and light chain (LC) sequences optimized and cloned into separate plasmids and transfected at expressed in transiently transfected ExpiCHO™ cells with standard reagents and harvested on Day 8. The protein was purified with MabSelect Sure™ (Cytiva) followed by Superdex 16/60 (Cytiva) and analyzed according to standard procedures known to one skilled in the art.

### Example V—Binding Characteristics of KB_A01

Binding of VHH KB_A01 to the hTfR1 and cTfR1 was identified during phage display panning towards hTfR1 and cTfR1, and the reactivity of the clone was confirmed by ELISA. Binding kinetics was analyzed further by surface plasmon resonance (SPR) and biolayer interferometry (BLI). VHH KB_A01 was tested for binding affinity to the TfR1 and for lack of interference in affinity by transferrin (Tf). SPR analyses were performed with on a Biacore T200 or Biacore 8K instrument (Cytiva, Sweden). SPR Series S Chip SA and Series S CM5 chips or Series S protein A chips (Cytiva, Uppsala Sweden) were used. Biotinylated transferrin was coupled to SA chip.

Binding characteristics of monomeric VHH KB_A01 along with the benchmark antibody BA1 were evaluated by SPR with a Biacore T200 instrument using single-cycle kinetics. Human Tf was amine-coupled on a CM5 chip (to about 3000 RUs) and subsequently hTfR1 was captured on immobilized human transferrin. Human TfR1 was loaded on hTf by injection at 100 nM followed by injection of increasing concentrations of analyte (VHH KB_A01 or BA1) ranging from 6.25 to 100 nM in 2-fold increments. The contact time was 150 s for each concentration at 30 μL/s with a final dissociation step of 500 s. At the end of each single cycle run, the hTf surface was fully regenerated by an injection of 10 mM glycine pH 2 for 30 s. Similarly, mTfR1 was amine-coupled on a CM5 chip to about 2200 RU and the same concentration-range of analyte (VHH KB_A01 or BA1) was applied as for the human TfR1 (6.25 to 100 nM in 2-fold increments) and the same SPR cycle as above but without the loading step was applied to these surfaces. At the end of each single cycle run, the mTfR1 surface was fully regenerated by an injection of 10 mM NaOH for 30 s. After each single cycle kinetic run, blank run was performed and used for baseline correction. The data with respect to the association rate constant (ka), the dissociation rate constant (kd) and the equilibrium dissociation constant (K_{D}) was evaluated using Biacore T200 software evaluation tool applying a Langmuir 1:1 model.

Monomeric KB_A01 and benchmark antibody BA1 bound to hTfR1 (Table 2 and FIG. 3) whereas none of the two antibodies bound to mTfR1. Since the hTfR1 was immobilized through loading on hTf, the binding of the analytes implies that neither KB_A01 nor BA1 competes with hTf for binding to hTfR1. Furthermore, the results from comparing KB_A01 to BA1 regarding affinity and binding kinetics showed that monomeric (free) KB_A01 bound to pre-complexed hTfR1-Tf with a 206-fold lower affinity (K_{D}) of 4.1 nM compared to the bivalent BA1 (K_{D}=0.0199 nM) owing to a dramatic difference in dissociation rate (kd). It has been shown that too high affinity may cause the VHH and any protein fused to it to be retained in the vasculature and/or cause lysosomal degradation of TfR1-anti-TfR1 complexes (Bien-Ly N, et al., Transferrin receptor (TfR) trafficking determines brain uptake of TfR antibody affinity variants. J Exp Med. 211(2):233-244 (2014)). Hence, VHH KB_A01 has binding characteristics that make it more suitable as drug transporter compared to BA1 due to its lower affinity to hTfR1 as compared to BA1.

TABLE 2
Affinity of monomeric VHH and full-length benchmark
antibody BA1 for hTfR1 and mTfR1 measured by SPR

| | TfR1 | | | |
|---|---|---|---|---|
| Clone | spec | ka (M⁻¹s⁻¹) | kd (s⁻¹) | K_{D} (M) |
| KB_A01 | Hum | 1.01 × 10⁶ | 4.14 × 10⁻³ | 4.10 × 10⁻⁹ |
| | Mouse | No binding | | |
| BA1 | Hum | 2.82 × 10⁶ | 5.63 × 10⁻⁵ | 1.99 × 10⁻¹¹ |

| | |
|---|---|
| Mouse | No binding |

### Example VI—Mapping of Epitopes of Monomeric KB_A01 Through Binning Experiments Against Known hTfR1-Binding Antibodies BA1, BA2 and BA3 by BLI

Recombinant KB_A01 was biotinylated with EZ-Link NHS-PEG4-Biotin biotinylation kit (ThermoFisher) according to manufacturer's instructions. Stock biotin solution vials were dissolved and stored at −70° C. in DMSO to a concentration of 20 nM, thawed and freshly diluted to 2 mM in ddH₂O and mixed with VHH protein (12 nmol-57 nmol protein) in equimolar ratio to biotin in PBS pH 7.4. PBS was added to 0.5 or 1 mL total volume. The mixture was incubated in a rotamixer at RT for 30 minutes followed by 4° C. overnight on a rocking table. Samples were concentrated using 500 μL spin concentrators (3000 MWCO at 13000 rpm for 5 min), repeated for certain samples until concentration was adequate. Samples were then desalted using NAP-5 column equilibrated with PBS pH 7.4 (Medicago) according to manufacturer's instructions. Briefly, samples were loaded and allowed to enter the resin, 400 μL PBS was added. Proteins were eluted by adding 500 μL PBS. Protein concentrations were estimated using absorbance at (lambda) 280. Yield was calculated for samples and ranged from 24% to 46%.

Analysis for hTfR1 binding and competition between KB_A01 and reference antibodies BA1, BA2 and BA3 was performed using BLI on an OctetRED96 instrument (ForteBio/Pall Life sciences). Samples were loaded on polypropylene 96-well microplates black (Greiner bio cat #655209). First, biotinylated KB_A01 (as a free VHH protein as described above) was immobilized at 2 μM on Streptavidin Dip and Read™ Biosensors for kinetics (Pall Life sciences, ForteBio). hTfR1 protein was added at 50 nM to each sensor, followed by regeneration with glycine pH 2 (5 s followed by 4 times 5 s in run buffer (Kinetics buffer 10× (Fortebio) diluted in PBS pH 7.4)), repeated in four cycles. On the last cycle, competing protein probes KB_A01 (control), BA1 and BA3 (purchased at Sigma Aldrich/Merck) were added at 10 nM and binding was recorded. Parameters for kinetic measurements were: baseline between steps 100 s, loading 300 s, quenching 1200 s, association 300 s, dissociation 200 s. Data was recorded and analyzed using Octet System Data Acquisition, Release 10.0 (ForteBio, Pall Life Sciences) and Octet System Data Analysis, Release 10.0—kinetics module (ForteBio, Pall Life Sciences). Resulting binding of biotinylated monomeric KB_A01 is shown in the first row of Table 3.

Next, full length benchmark antibodies BA1, BA2 and BA3 were immobilized on protein A (ProA) Dip and Read Biosensors for kinetics (Pall Life sciences, Fortebio) at 1 μM. hTfR1 (50 nM) was added to each biosensor according to the same principle as above, followed by competing probes, VHH KB_A01 (unbiotinylated) or antibodies BA1 or BA3 (all at 10 nM). Owing to the setup of the loading on protein A, the monoclonal antibodies could not be run against one another (labeled nd=not done in Table 3). As indicated in Table 3 (rows 2-4), KB_A01 blocked BA1 from binding to hTfR1. In contrast, KB_A01 and benchmark antibody BA2 as well as KB_A01 and BA3 could bind simultaneously to hTfR1. Thus, KB_A01 binds to a specific epitope overlapping with, but not identical to that of BA1. BA1 and antibody BA3 are known to bind to overlapping epitopes of the apical domain and, thus, compete for binding to hTfR1 (Sade et al. A Human Blood-Brain Barrier Transcytosis Assay Reveals Antibody Transcytosis Influenced by pH-Dependent Receptor Binding, PLOS one 4(1): e936340 (2014)). Thus, we can further conclude that KB_A01 binds to the apical domain as well.

TABLE 3
Epitope binning of monomeric VHH KB_A01
against benchmark antibodies using BLI

| Immobilized ligand | KB_A01 | BA1 | BA3 |
|---|---|---|---|
| KB_A01 | X | B | — |
| Antibody BA1 | B | X | nd |
| Antibody BA2 | — | nd | nd |
| Antibody BA3 | — | nd *(B) | X |

B = blocking, — = not blocking, nd = not determined, X = autologous competition (against self).
*(B) = reported in literature

### Example VII—Characterization of Fc-Fused VHHs

Affinity measurements were carried out by SPR on a Biacore T200 (Cytiva), by loading KB_A01-Fc fusion, BV (Fc fusion) and BA2 (Example IV) on protein A chip (Cytiva) at a high degree (100 nM for 60 s). Subsequently it was exposed to increasing concentrations of hTfR1 ranging from 0.16 to 100 nM in 5-fold increments. The data is summarized in Table 4 and sensorgrams are presented in FIG. 4. The dimeric, KB_A01-Fc bound to hTfR1 with a K_{D} value of 0.177 nM (Table 4), i.e., 23-fold higher affinity compared to the free monomeric KB_A01 binding to immobilized hTfR1 (K_{D}=4.1 nM see Table 2). As controls, benchmark BV (VHH-Fc) and BA2 (IgG1) were run under the same conditions and displayed affinities (K_{D} values) of 0.00228 and 0.0249 nM, respectively. Mouse TfR1 was also run under the same conditions and neither KB_A01-Fc nor benchmark antibody BA2 bound mTfR1 with detectable signals. In contrast, the benchmark VHH-Fc (BV) bound mTfR1 with high affinity (K_{D}) of 0.444 nM (FIG. 4D). These experiments show that KB_A01 has desirable binding characteristics of nM affinity range and also an interesting profile of K_{D} in terms of release (OFF) to the receptor. KB_A01 in contrast to BV only binds hTfR1 and not mTfR1.

TABLE 4
Binding kinetics of KB_A01 as dimeric Fc fusion
and controls to human or mouse TfR1 using SPR

| Clone | TfR1 species | ka (M⁻¹s⁻¹) | kd (s⁻¹) | K_{D} (M) |
|---|---|---|---|---|
| KB_A01 | Human | 4.76 × 10⁵ | 8.49 × 10⁻⁵ | 1.77 × 10⁻¹⁰ |
| | Mouse | No apparent binding | | |
| BV | Human | 5.51 × 10⁵ | 1.26 × 10⁻⁶ | 2.28 × 10⁻¹² |
| | Mouse | 3.82 × 10⁵ | 1.70 × 10⁻⁴ | 4.44 × 10⁻¹⁰ |
| Antibody BA2 | Human | 1.56 × 10⁶ | 3.89 × 10⁻⁵ | 2.49 × 10⁻¹¹ |

| | |
|---|---|
| Mouse | No apparent binding |

To further investigate the effect of soluble human transferrin on binding to human and cynomolgus monkey TfR1, SPR as above was performed in the presence and absence of human Tf. Mouse TfR1 was also included as a control. KB_A01-Fc, BV-Fc, BA1 and BA2 were immobilized on protein A sensors (Cytiva) and subsequently exposed to increasing concentrations ranging from 0.156 to 40 nM (4-fold increments) of the receptors in the absence or presence of a saturating concentration of 250 nM hTf. There was little difference when comparing the affinity in the absence or presence of 250 nM of hTf suggesting that KB_A01, and the two benchmark antibodies BA1 and BA2 do not interfere with Tf binding to the human or cynomolgus TfR1s (Table 5, FIGS. 5A-6D and 6A-6D). However, there was a significant difference in affinity (K_{D}) to human TfR1 for BV-Fc in the presence or absence of hTf. Furthermore, data demonstrated a high affinity for cynomolgus monkey TfR1, whereas no binding to mouse TfR1 was recorded (Table 5). This experiment shows that KB_A01 has the desirable and unique binding characteristics for using the TfR1 molecule in non-human primates and man to increase delivery to the CNS.

TABLE 5
Effect of the presence of transferrin on binding
to human and cynomolgus monkey TfR1

| | TfR1 | | | | |
|---|---|---|---|---|---|
| Clone | spec | hTf | ka (M⁻¹s⁻¹) | kd (s⁻¹) | K_{D} (M) |
| KB_A01- | Hum | No | 8.01 × 10⁵ | 6.49 × 10⁻⁵ | 8.10 × 10⁻¹¹ |
| Fc | | 250 nM | 2.93 × 10⁵ | 6.03 × 10⁻⁵ | 2.06 × 10⁻¹⁰ |
| | Cyn | No | 1.86 × 10⁵ | 7.50 × 10⁻⁵ | 4.03 × 10⁻¹⁰ |
| | | 250 nM | 1.51 × 10⁵ | 2.86 × 10⁻⁵ | 1.89 × 10⁻¹⁰ |
| | Mouse | No apparent binding | | | |
| BA2 | Hum | No | 3.15 × 10⁶ | 5.65 × 10⁻⁵ | 1.80 × 10⁻¹¹ |
| | | 250 nM | 1.07 × 10⁶ | 1.42 × 10⁻⁴ | 1.33 × 10⁻¹⁰ |
| | Cyn | No | 1.54 × 10⁵ | 7.86 × 10⁻⁵ | 5.10 × 10⁻¹⁰ |
| | | 250 nM | 3.62 × 10⁵ | 1.18 × 10⁻⁴ | 3.24 × 10⁻¹⁰ |
| | Mouse | No apparent binding | | | |
| BA1 | Hum | No | 8.82 × 10⁵ | 8.87 × 10⁻⁵ | 1.01 × 10⁻¹⁰ |
| | | 250 nM | 6.92 × 10⁵ | 5.77 × 10⁻⁵ | 8.34 × 10⁻¹¹ |
| | Cyn | No | 2.14 × 10⁵ | 8.85 × 10⁻⁵ | 4.14 × 10⁻¹⁰ |
| | | 250 nM | 2.42 × 10⁵ | 8.00 × 10⁻⁵ | 3.30 × 10⁻¹⁰ |
| | Mouse | No apparent binding | | | |
| BV-Fc | Hum | No | 5.38 × 10⁵ | 2.86 × 10⁻⁷ | 5.31 × 10⁻¹³ |
| | | 250 nM | 5.49 × 10⁵ | 1.13 × 10⁻⁴ | 2.07 × 10⁻¹⁰ |

### Example VIII—Bioinformatics and Analysis of Additional Clones

KB_A01 was aligned to all the clones sequenced based on initial screening of clones (panning) using Jalview multiple sequence alignment editor and analysis workbench version 2 (Barton group, University of Dundee) software. Based on sequence identity to KB_A0l, and confirmed positive binding in ELISA, three additional clones, KB_A09, KB_A10, and KB_A11 (see FIG. 7 for alignment) were selected and produced as fusions to human IgG1 Fc (Example IV). Next, combinations of CDRs were designed, where CDRs from one sequenced clone (KB_ref, SEQ ID NO: 53) were transplanted into the framework of KB_A01 generating KB_A03, KB_A04, KB_A06 and KB_A07 where CDR2 (KB_A03), CDR3 (KB_A04), CDR1 and CDR3 (KB_A06), and CDR2 and CDR3 (KB_A07) have been replaced in KB_A01. Replacement of CDR1 alone (KB_A02) in KB_A01 or in combination with CDR2 (KB_A05) or with CDR2 and CDR3 (KB_A08) resulted in VHHs incapable of binding hTfR1 as shown in Table 7.

TABLE 6
CDRs of KB_A01 and KB_A02 to KB_A08

| VHH | CDR1 | CDR2 | CDR3 |
|---|---|---|---|
| KB_A01 | GSIFGSKR¹ | ITYRGTT³ | WMFTTDNY5 |
| KB_A02 | GSIFGFNA² | ITYRGTT | WMFTTDNY |
| KB_A03 | GSIFGSKR | IAVAGST⁴ | WMFTTDNY |
| KB_A04 | GSIFGSKR | ITYRGTT | WMYATANY⁶ |
| KB_A05 | GSIFGFNA | IAVAGST | WMFTTDNY |
| KB_A06 | GSIFGFNA | ITYRGTT | WMYATANY |
| KB_A07 | GSIFGSKR | IAVAGST | WMYATANY |
| KB_A08 | GSIFGFNA | IAVAGST | WMYATANY |

¹SEQ ID NO: 1
²SEQ ID NO: 2
³SEQ ID NO: 3
⁴SEQ ID NO: 4
⁵SEQ ID NO: 5
⁶SEQ ID NO: 6

Based on the above KB_A02-KB_A11 were produced as fusions to human IgG1 Fc (see Example IV, SEQ ID NO: 62-71) and tested for affinity for the hTfR1 and mTfR1 using SPR and immobilizing the dimeric Fc-fused VHHs to protein A chips as described above. Briefly, using Biacore 8K and single-cycle kinetics, VHH-Fc constructs were diluted to 5 nM and captured on protein A chip (Cytiva), eight at a time for 120 s at 5 μL/s followed by injection of increasing concentrations of receptors ranging from 0.8 to 500 nM (contact time 120 s, dissociation 600 s) in five-fold increments. After each full single-cycle kinetics cycle, the surfaces were regenerated by 10 mM glycine, pH 1.5 for 30 s at 30 μL/s. Kinetic data for KB_A02 to KB_A11 as Fc fusions are presented in Table 7. KB_A03 KB_A04 KB_A06 KB_A07 KB_A09, KB_A10, KB_A11 all bound hTfR1 with K_{D} values ranging from 0.150 to 5.12 nM (Table 7). In contrast, KB_0A2, KB_A05 and KB_A08, when immobilized on protein A, did not bind hTfR1. No binding to hTfR1 was seen for KB_ref fused to human IgG1 Fc (SEQ ID NO: 85).

TABLE 7
Binding kinetics of dimeric Fc fusions with
KB_A02 to KB_A11 to human TfR1 using SPR

| Clone | ka (M⁻¹s⁻¹) | kd (s⁻¹) | K_{D} (M) | Rmax (RU) |
|---|---|---|---|---|
| KB_A02 | No apparent binding | | | |
| KB_A03 | 2.74 × 10⁵ | 9.92 × 10⁻⁴ | 3.62 × 10⁻⁹ | 829.5 |
| KB_A04 | 2.87 × 10⁵ | 1.19 × 10⁻⁴ | 4.14 × 10⁻¹⁰ | 822.6 |
| KB_A05 | No apparent binding | | | |
| KB_A06 | 6.90 × 10⁴ | 3.53 × 10⁻⁴ | 5.12 × 10⁻⁹ | 363.8 |
| KB_A07 | 1.54 × 10⁵ | 1.08 × 10⁻⁴ | 6.99 × 10⁻¹⁰ | 643.9 |
| KB_A08 | No apparent binding | | | |
| KB_A09 | 4.05 × 10⁵ | 6.09 × 10⁻⁵ | 1.50 × 10⁻¹⁰ | 1095.6 |
| KB_A10 | 5.94 × 10⁴ | 1.10 × 10⁻⁴ | 1.84 × 10⁻⁹ | 505.6 |
| KB_A11 | 3.62 × 10⁵ | 8.76 × 10⁻⁵ | 2.42 × 10⁻¹⁰ | 866.0 |

| | |
|---|---|
| KB_ref | No apparent binding |

Next, epitope binning was performed by pairwise testing binders for the hTfR1 in a combinatorial manner using SPR on a Biacore 8K. KB_A01 and KB_A09 along with benchmark antibodies BA1, BA2 and benchmark VHH-Fc (BV) were immobilized by amine coupling onto CM5 chips series S (Cytiva). Proteins to be immobilized were diluted to 25 μg/mL in sodium acetate at pH 5.5 and bound proteins to a SPR signal of about 6000 resonance units (RUs). 50 nM of the ectodomain of hTfR1 was used for the first injection (contact time 120 s, 10 μL/s) and 100 nM of each analyte in the second injection; KB_A01, KB_A03, KB_A04, KB_A09, KB_A10, KB_A11, BA1, BA2, BV, mouse transferrin (mTf, purified, Jackson Immunoresearch Europe Ltd) and human transferrin (hTf, purified, Jackson Immunoresearch Europe Ltd) over a contact time of 150 s, 10 μL/s. Following each cycle, the surfaces were regenerated by 10 mM glycine at pH 2.1 for 30 s at 30 μL/s. The data were analyzed using Biacore Insight Evaluation package version 4.0.8.20368. Results showed that KB_A03, KB_A09, KB_A10 and KB_A11 all competed with KB_A01, KB_A09 and antibody BA1 (Table 8) whereas none did so with BA2 or with BV. Antibody BA1, known to bind to the apical domain of the hTfR1 (Helguera G. et al., An antibody recognizing the apical domain of human transferrin receptor 1 efficiently inhibits the entry of all new world hemorrhagic Fever arenaviruses. J Virol. 86(7):4024-4028 (2012)), competed with BA2 as well as the above mentioned VHH-Fc fusion constructs but not BV. Quite surprisingly, KB_A04 did not compete with BA1 (Table 8). Thus, KB_A01, KB_A03, KB_A09, KB_A10 and KB_A11 form a separate bin, partly but not entirely overlapping with that of KB_A04 and that of BA1. The control VHH-Fc molecule BV did neither block any of the other anti-TfR1 molecules nor mTf and hTf from binding to hTfR1. Hence, the VHH antibody of BV binds to an epitope totally distinct from that of KB_A01, KB_A03, KB_A09, KB_A10 and KB_A11.

TABLE 8
Epitope binning of dimeric VHH-Fc fusions and benchmark antibodies using SPR

| Immobilized | KB_{—} | KB_{—} | KB_{—} | KB_{—} | KB_{—} | KB_{—} | | | | | |
|---|---|---|---|---|---|---|---|---|---|---|---|
| ligand | A01 | A03 | A04 | A09 | A10 | A11 | BA1 | BA2 | BV | mTf | hTf |
| KB_A01 | X | B | B | B | B | B | B | — | — | — | — |
| KB_A09 | B | B | B | X | B | B | B | — | — | — | — |
| BA1 | B | B | — | B | B | B | X | B | — | — | — |
| BA2 | — | — | — | — | — | | B | X | — | — | — |
| BV | — | — | — | — | — | — | — | — | X | — | — |

B = Blocking, — = non-blocking, X = autologous competition (against self).

In order to prove functionality when fused to a non-Fc protein, KB_A01 was also produced as a fusion to a single-chain variable domain (scFv) located either in the N-terminal (scFv-KB_A01) or C-terminal (KB_A01-scFv) see Example IV and FIG. 2 for details. In order to verify that these fusion proteins still bound to hTfR1, binding kinetics was studied by SPR using an 8K Biacore, Briefly, the receptor hTfR1 was immobilized to a CM5 chip (Cytiva) by amine-coupling and exposed to increasing concentrations of either scFv-KB_A01 or KB_A01-scFv ranging from 0.25 to 64 nM in 4-fold increments using single-cycle kinetics. The kinetic binding data and sensorgrams are presented in Table 9 and FIG. 8.

TABLE 9
Binding kinetics of KB_A01 fusions
to a scFv human TfR1 using SPR

| Construct | ka (M⁻¹s⁻¹) | kd (s⁻¹) | K_{D} (M) | Rmax (RU) |
|---|---|---|---|---|
| scFv-KB_A01 | 1.70 × 10⁶ | 5.39 × 10⁻³ | 3.16 × 10⁻⁹ | 56.1 |
| KB_A01-scFv | 2.18 × 10⁶ | 7.50 × 10⁻³ | 3.44 × 10⁻⁹ | 51.8 |

### Verification of Apical Domain Binding of KB_A01-Family Binders by ELISA

To further assess whether KB_A01 and related anti-hTfR1 VHHs bind within the apical domain of the hTfR1, we expressed the apical domain through display on M13 phages. It is known that the apical domain expressed as a single soluble domain is unstable, why this approach was set. A synthetic gene encoding a recombinant variant of the apical domain of the hTfR1 (covering regions Q191-F297 and S326-V380 of full length hTfR, corresponding to Q80-F186 and S215-V269 of the ectodomain of hTfR, SEQ ID NO: 30), with introduced mutations at Y222S and L329S to increase solubility, was provided by Genscript Biotech Corp (SEQ ID NO: 54), see SEQ ID NO: 72 for amino acid sequence. The gene was flanked by the restriction enzyme sites AgeI and NotI. Using restriction digestion and T4 ligation (New England Biolabs), the AP1 gene was cloned into the phagemid pTG3. The resulting phagemid pTG3-AP1 was transformed via heat shock into E. coli XL1-blue and plated on LA-Ampicillin (100 μg/ml) plates. Transformants were grown in 2×YT medium supplemented with ampicillin (100 μg/ml) and the phagemid mini-prepped (Monarch Plasmid Mini Prep Kit, New England Biolabs). The sequence of the pTG3-AP1 phagemid prep was confirmed using Sanger sequencing (Eurofins Genomics GmbH).

E. coli XL1-blue transformed with pTG3-AP1 was cultured in 30 ml 2×YT medium supplied with 5 μg/ml tetracycline and 100 μg/ml ampicillin at 37° C. until OD₆₀₀ reached 0.7. Helper phage M13K07 0.5 ml (3.1×10¹¹ pfu/ml) was added to the culture followed by 1 h incubation at 37° C. Kanamycin 50 μg/ml and IPTG 0.25 mM were added to the cultured followed by incubation at 30° C. overnight. The culture was centrifuged, and the supernatant transferred to a new tube. 7.5 ml NaCl-PEG (20% PEG-8000/2.5 M NaCl) was added to the supernatant followed by incubation on ice for 30 minutes. The supernatant was centrifuged at 4° C. for 30 minutes. The supernatant was discarded, and the phage pellet dissolved in 2 ml 1% BSA PBS. The resuspended phage preparation was centrifuged briefly to remove larger aggregates, followed by a 0.2 μm filtration.

An immunoplate (Nalge Nunc International) was coated using the constructs KB_A01-Fc and KB_A02 to KB_A11 including benchmark antibodies BA2 and BV-Fc, to be tested at a concentration of 10 μg/ml (5 μg/ml for Fc-fusions) in 0.05 M sodium carbonate buffer pH 9.4. The plate was incubated overnight at 4° C. The plate was washed twice in PBS-tween. Blocking buffer 1% BSA in PBS was added and incubated for 1 hour at RT followed by washing the plate twice in PBST. M13 phage displaying hTfR1-apical (SEQ ID NO: 54) were added in a dilution series starting with a dilution of 1:1 in PBST. For the ectodomain of hTfR1 (SEQ ID NO: 30), the plate was coated using hTfR1 at 10 μg/ml and antibodies were added in dilution series as above. The plate was incubated for 2 hours at RT followed by washing four times using PBST. Anti-M13-HRP antibody (#11973-MM05T-H, Sino Biological) or anti-Fc HRP (Pierce #31423, 1 mg/mL) both diluted 1:5000 in PBS-tween was added followed by an incubation of 1 hour at RT. The plate was washed four times using PBST where after 1-Step™ Ultra TMB-ELISA Substrate Solution (ThermoFisher Scientific) was added and developed for 30 minutes at RT. The reaction was stopped using 2 M H₂SO₄ and the absorbance at 450 nm was measured. This set of experiments showed that KB_A01, KB_A04, KB_A09, and KB_A11 and BA2 bound to the apical domain, but not BV (FIG. 8A and FIG. 8B). In control experiments with the ELISA method described above, both KB_A01, BA2 and BV confirmed to bind the ectodomain of the hTfR1 with the same methodology (FIG. 8C), which also has been confirmed in all SPR experiments above.

Together with experiments above relating to binning, we can conclude that KB_A01, KB_A03, KB_A04, KB_A07. KB_A09. KB_A11 bound to the same limited region of the apical domain of the hTfR1. Although not tested herein, it is highly unlikely that KB_A07, which has sequence homology with the other apical binding VHH clones and blocks these in binning experiments, would not bind the apical domain. In addition, the benchmark VHH antibody in BV was found not to bind the apical domain of the hTfR1.

### Example IX—TfR1 Mediated Uptake in Human Cells

Next, it was tested whether the KB_A01 VHH-Fc-fusion protein could internalize in TfR1-expressing cells. Adherent HEK293T cells (ATCC), well known to express TfR1, were grown in collagen-coated 96 well plates (ThermoFischer Scientific), seeded at a density of 30.000 cells/well. Cells were grown in DMEM (Dulbecco's modified eagle medium) for 3-5 days, until about 80-90% confluence. KB_A01-Fc as well as known reference binders BA1, BA2 and a negative control (an analogous VHH-Fc known not to bind hTfR1) were used in this assay.

The cell assay was established based on a titration of test items KB_A01, BA1, BA2 and negative control ranging from 0.85 nM to 82.5 nM during 5-15-30-45 minutes, in 37° C., 5% CO₂. For comparing cellular fate of added proteins after a prolonged exposure, a 30 min incubation was followed by medium replacement by fresh DMEM for 90 minutes (incubated in 37° C., 5% CO₂) followed, after which the medium was replaced a second time. After 120 min, cells were taken to lab bench and immediately washed with PBS (1×) and fixed with 4% paraformaldehyde for 10-15 minutes. After fixation, cells were washed with PBS and permeabilized with 0.1% Triton-X 100™ in PBS and thereafter blocked with 1% bovine serum albumin (BSA) in PBS, followed by addition of solution Alexa Fluor-488-anti human IgG (Fc fragment specific, stock 0.75 mg/ml) (Jackson Immunoresearch). Texas Red-phalloidin 300 U (1:200 dilution of a 10 μg/mL stock) was added to the wells during the last 20 minutes of incubation of secondary antibody. After this, cells were washed at least 3 times with PBS and stored at 4° C. until imaging. Cells were imaged in an inverted Zeiss 710 laser scanning confocal microscope using lasers 488, 555 and 647 and a 20× air objective.

Results from these experiments showed that KB_A01 as well as BA1 and BA2 were readily taken up into HEK293T cells at various concentrations tested (0.7-85 nM) and at incubation times 5 to 240 minutes. As shown in FIG. 10A, KB_A01-Fc was taken up efficiently into cells similarly to BA1. At the shorter timepoints 15-45 minutes, the amounts were equivalent of KB_A01-Fc and BA1. At the longer incubation timepoints, BA1 had a clearly reduced intracellular presence compared to both KB_A01 and BA2 (FIG. 10C), i.e., was less abundant in cells after 240 minutes, indicating lysosomal degradation of BA1. A negative control (VHH-Fc, devoid of binding to hTfR1) did not accumulate in cells and did not show any presence at any timepoint or concentration tested, showing that the assay and uptake was TfR1-dependent.

### Example X—Evaluation of Transcytotic Potential

An experiment was conducted to evaluate the transcytotic potential of KB_A01 and compared with a selected reference IgG and benchmark TfR1-binding BA2. An in vitro BBB transwell assay with brain-like endothelial cells was performed in a tight monolayer on a transwell system as described in Sjöström et al., Transport study of interleukin-1 inhibitors using a human in vitro model of the blood-brain barrier, Brain Behaviour, Immunity Health 16: 100307 (2021). The assay was performed at Laboratoire de la barrière Hématoencéphalique (LBHE), Université d'Artois, France. To evaluate the transcytotic potential of VHH-Fc fusions, 500 nM of test compounds (VHH-Fcs or full-length antibodies) were added to donor wells in triplicates and incubated at 37° C. for three hours. At the end of the experiment, all compartments were harvested. Cells were lysed and the contents from all compartments (donor, cells and receiver chamber) were analyzed with ELISA reactive to human Ig (Fc specific) from Mabtech, Sweden. The plates were coated with anti-human IgG mAb MT145 (Fc specific) and blocking was performed with 0.1% BSA in PBST. Sample and standard dilutions were added. The detection reagents were then added in two steps, first biotinylated anti-human IgG mAb MT78 (Fc specific) and then HRP conjugated streptavidin. Washing was performed with PBST between each step. An equal volume of TMB was added and finally the reaction was stopped with 2 M HCl and the plates were read. The substrate incubation time was set to 10 min. As a reference, a control monoclonal antibody devoid of active transcytotic activity (reactive to interleukin 1-beta) was used. This control monoclonal antibody does not bind the TfR1 and has low BBB passage. Furthermore, BA2 a clinically validated transporter was used (Example IV). The method was verified by internal controls in all wells, through co-incubation and measurement of human serum albumin (HSA) coupled to fluorescent Alexa-647 as well as sodium fluorescein (NaF), to control for the tightness of monolayers. All wells included in the comparison were tight and showed similar rates of HSA distribution across the polarized cell monolayer.

As depicted in FIG. 11, KB_A01 VHH-Fc showed clearly superior ability to transcytose and remain in the abluminal compartment of the polarized cell monolayer. This experiment shows that KB_A01 as VHH-Fc fusion had the ability of directed transcytosis through a human polarized brain-like endothelial cell monolayer.

### Example XI—In Vivo and Ex Vivo Evaluation of VHH-Fc of KB_A01 in TfR1 Extracellular Domain Humanized Mice (hECD-TfR1-Mice)

To evaluate the properties of KB_A01 for brain targeting in vivo, the distribution properties of KB_A01 was tested in hECD-TfR1-mice of homozygous, heterozygous and wild type genotype. Homozygous mice only express the partially humanized TfR1, i.e., human-mouse chimeric TfR1. Humanized mice expressing a chimeric receptor comprising the extracellular domain of hTfR1 and the intra- and transmembrane domains of mTfR1 were generated at Taconic Biosciences GmbH, Leverkusen, Germany.

A genetically engineered mouse model was generated by Taconic Biosciences GmbH, Leverkusen, Germany, in which the endogenous murine Tfrc gene was partially humanized so that the engineered mice produced a chimeric TFRC protein containing the human TFRC extracellular domain. The targeting vector was assembled using methods known to one skilled in the art using BAC-derived mouse and human genomic DNA fragments and including standard selection cassettes.

Embryonic stem cells (ES) derived from C57BL/6NTac mice, were grown on a mitotically inactivated feeder cell layer comprised of mouse embryonic fibroblasts in ES cell culture medium containing leukemia inhibitory factor and fetal bovine serum. The cells were electroporated with the linearized DNA targeting vector according to methods known to a person skilled in the art. Homologous recombinant clones were isolated using methods known to one skilled in the art. ES cell colonies (ES clones) with a distinct morphology were isolated and analyzed by Southern blotting and/or PCR. Homologous recombination at 3′ and 5′ sites and single integration were confirmed with PCR and Southern blot using conventional methods, known to the one skilled in the art. Homologous recombinant ES cell clones were expanded and frozen in liquid nitrogen.

Generation of chimeras and generation of heterozygotes: After administration of hormones, superovulated BALB/c females were mated with BALB/c males. Blastocysts were isolated from the uterus at dpc 3.5 and placed in a drop of DMEM with 15% FCS (fetal calf serum) under mineral oil. A microinjection-pipette was used to inject 10-15 targeted ES cells into each blastocyst. After recovery, approximately 8 injected blastocysts were transferred to each uterine horn of pseudopregnant NMRI females. The degree of chimerism was assessed in chimeras (F0) by observing coat color contribution of ES cells to the BALB/c host (black/white). In vitro fertilization was performed using oocytes from superovulated C57BL/6NTac females and thawed sperm from previously cryopreserved spermatozoa derived from male chimeras. Fertilized embryo were then transferred into pseudopregnant Swiss Webster recipient females. Germline transmission was identified first by the presence of offspring with black coats (strain C57BL/6NTac) and the confirmed by genotyping of the black offspring via PCR. F1 mice heterozygous for the modified Tfrc allele were mated to produce subsequently homozygous (HOM), heterozygous (HET) and wild type (WT) genotypes, that were used for the studies described below.

KB_A01-Fc was radiolabeled with iodine-125 (¹²⁵I) using the chloramine T method as described in Greenwood et al., The preparation of ¹³¹I-labelled human growth hormone of high specific radioactivity. Biochem J 89: 114-123 (1963). An amount of 15-80 μg of protein was mixed with 260±26 MBq/μg or 61±2.4 kBq/μg of stock ¹²⁵I (PerkinElmer Inc, Waltham, MA, USA) and 5 μg of Chloramine T (Sigma Aldrich) in PBS. The reaction mixture (110 μL) was allowed to incubate for 90 s before it was quenched with 10 μg of Na-metabisulfite (Sigma-Aldrich). The radiolabeled sample was immediately purified in a Zeba-column (ThermoFischer) of 7 kDA cutoff. Experiments in vivo were performed in C57BL/6 mice aged 6-10 weeks. Immediately following radiolabeling, mice were administered KB_A01-Fc (5 nmol/kg; 3.4 MBq/nmol) i.v. via the tail vein. Blood samples (8 μL) were obtained from the tail vein at 5 min, 0.5 h, 1 h and terminally at 2 hours post injection. Mice were euthanized 2 hours post injection by sampling blood from the heart prior to transcardial perfusion with 40 mL NaCl during 2.5 min. Brain and major organs were dissected. Blood was separated into plasma and blood cell pellet by centrifugation at 10 000× g, 5 min. The brain was separated into the left and right hemispheres. The cerebellum was removed from the left hemisphere, and the remaining tissue of the left hemisphere is hereafter referred to as “brain”. Radioactivity was then measured in brain, blood fractions and major organs using a γ-counter (2480 Wizard™, Wallac Oy PerkinElmer, Turku, Finland). As shown in FIGS. 13A and 13B, pharmacokinetic blood profile showed clearance from blood highest in HOM mice followed by HET mice, while lowest in WT mice. Further, distribution between whole blood, pellet and plasma, showed highest presence in the blood pellet (after spinning whole blood as described above) in HOM mice, followed by HET mice, while lowest in WT mice, indicating binding to hTfR1 on red blood cells in vivo. FIG. 13C shows standardized brain uptake (SUV), which was substantially higher in HOM mice compared to that of WT mice, while close to 50% less in HET mice than HOM mice. This clearly shows the specific brain targeting capacity of KB_A01 in a functional hECD-TfR1 in vivo.

### Example XII—Epitope Characterization

The binding sites of KB_A01, KB_A03 and KB_A09 in the form of Fc-fusions as described in Example IV along with the two benchmark antibodies BA1, BA2 were investigated to determine the exact contribution of amino acids in the apical domain of the human TfR1. The epitope mapping was performed using Seqitope™ (AAX Biotech AB, Sweden) based on massive parallel mutagenic scanning. Seqitope™ outputs a Binding Ratio (BR) value for each amino acid residue of the target antigen, which correlates to the importance of that amino acid residue in binding of the antibody. A high BR value (>2.5) indicates a high contribution of this amino acid residue in the binding of the antibody, though this value seems to vary depending on the affinity of the investigated antibody. A BR value of 1.0 indicates no contribution of that amino acid residue to the binding of the antibody.

The target antigen was the apical domain of the hTfR1 expressed on M13 phages as described in Example VIII.

KB_A01, KB_A03 and KB_A09 and the tested benchmark antibodies BA1 and BA2 bound to the apical domain expressed on M13 phages while benchmark BV did not.

The epitopes for the benchmark antibodies BA2 on hTfR1 (D204, K205, N206, R208, V210, E369) and BA1 (D242, L246, P249, E369, V366, G351, R364) and the antibodies KB_A01 (P354, L246, D245, G351, D242, R364, S355, D356), KB_A03 (D356, P354, D242, D245, G351, L246, S355) and KB_A09 (D356, D245, P354, G351, D242, L246, S355) were resolved in this example (see Table 10). The antibodies within the KB_A01-group (KB_A01, KB_A03 and KB_A09) bound to similar epitopes of the apical domain.

The detailed resolution of the epitopes obtained by Seqitope™ revealed that the epitope, to which BA1 bound, was partially overlapping with the epitope, to which BA2 bound (E369), but also with the epitopes, to which the antibodies in the KB_A01-group (D242, L246 and G351) bound. The conclusion is that the epitope, to which the KB_A01-group bound, is distinct from, but partially overlapping with the BA1 epitope and does not overlap at all with the BA2 epitope. This agrees with the binning data in Example VIII where KB_A01, KB_A03, KB_A09 and BA1 all competed with one another, BA1 and BA2 competed with each other while BA2 did not compete with the KB_A01-group members. However, the Seqitope™ data provided further detail and showed a differentiation of present antibodies as compared to the benchmark antibodies. The benchmark VHH BV was not included as it had already been shown not to bind the apical domain (see FIG. 8A). The epitope for BA1 has been mapped to a stretch of the apical domain between Ser324 and Ser368 where three of the identified amino acids are located (Helguera G. et al., An antibody recognizing the apical domain of human transferrin receptor 1 efficiently inhibits the entry of all new world hemorrhagic Fever arenaviruses. J Virol. 86(7):4024-4028 (2012)).

Although the epitopes for the three VHH-Fc variants tested overlapped to a large extent, the individual contribution of each amino acid of hTfR1 varied somewhat. Most surprisingly, D356 contributed most strongly to binding of KB_A03 and KB_A09 whereas this amino acid was the one most weakly associated with binding of KB_A01.

TABLE 10
Epitopes based on Seqitope ™ data

| KB_A01 | | KB_A03 | | KB_A09 | | BA1 | | BA2 | |
|---|---|---|---|---|---|---|---|---|---|
| aa | BR | aa | BR | aa | BR | aa | BR | aa | BR |
| P354 | 2.45 | D356 | 2.24 | D356 | 3.95 | E369 | 6.88 | V210 | 5.46 |
| D245 | 2.20 | P354 | 1.94 | D245 | 3.59 | V366 | 4.15 | R208 | 5.24 |
| L246 | 2.20 | D242 | 1.76 | P354 | 3.51 | D242 | 3.56 | K205 | 5.09 |
| G351 | 2.14 | D245 | 1.64 | G351 | 3.20 | L246 | 3.36 | D204 | 4.13 |
| D242 | 2.08 | G351 | 1.56 | D242 | 2.87 | G351 | 3.27 | N206 | 4.07 |
| R364 | 1.74 | L246 | 1.55 | L246 | 2.82 | R364 | 2.64 | E369 | 3.50 |
| D356 | 1.42 | S355 | 1.54 | S355 | 2.24 | P249 | 2.64 | | |

### Example XIII—Generation of Protein A Binding Antibodies

Llama VHHs do not usually bind the protein A based resin PrismA™ but by mutating amino acids in the framework sequences, PrismA™ binding, and thereby protein A binding, VHHs can be produced. To explore the possibility of enabling a protein A binding of KB_A01, KB_A09, and KB_A11 and fusion proteins containing KB_A01, KB_A09, and KB_A11, these three VHHs were mutated according to consensus protein A binding sequences according to Henry et al., A Rational Engineering Strategy for Designing Protein A-Binding Camelid Single-Domain Antibodies, PLoS One 11(9): e0163113 (2016). KB_A01 was mutated in position 19 from a serine to an arginine and in position 84 from an asparagine to a serine (KB_A01: S19R, N84S, denoted KB_A12 SEQ ID NO: 73), KB_A09 was mutated in in position 19 from a serine to an arginine (KB_A09: S19R, denoted KB_A13 SEQ ID NO: 74), and KB_A11 was mutated in position 19 from a serine to an arginine (KB_A11: S19R, denoted KB_A14 SEQ ID NO: 75). A positive control (positive control 1) known to bind protein A (SEQ ID NO: 76) was also included. As expression control, Green Fluorescent Protein (GFP) was expressed, and can be seen on blots in FIG. 12, but was not incorporated in any other analyses. All variants were fused to a His6-tag via a short Gly2 linker (GGHHHHHH, SEQ ID NO: 77) to enable Ni-NTA based purification. Plasmids (pNIC28_Bsa4) with codon optimized sequences were ordered from GeneArt Thermo Fisher and used to transfect E. coli BL21 (DE3) pRARE2 cells at the Protein Science Facility, Karolinska Institute, Sweden according to standardized procedures. Briefly, pre-cultures were grown overnight and used to inoculate expression cultures. The expression cultures were cultured at 37° C. in 2×1 ml Terrific Broth supplemented with 8 g/l glycerol and appropriate antibiotics. On day two, the temperature was lowered to 18° C. and the cells were induced with 0.5 mM isopropyl β-D-1-thiogalactopyranoside (IPTG) an hour later. Protein expression continued overnight at 18° C. before the cells were harvested by centrifugation (20 min at 4000×g). The cells were stored at −80° C.

The cells were resuspended in lysis buffer (100 mM HEPES, 500 mM NaCl, 10% glycerol, 10 mM imidazole, 0.5 mM TCEP, pH 8.0.1 mg/ml lysozyme, 0.1% DDM, 1 mM MgSO₄, Protease inhibitor (Roche cOmplete™ EDTA-free 0.5 tab/ml), Benzonase (0.125 U/mL)) and lysed by freezing and thawing three times. The total cell lysates were clarified by centrifugation (4000×g, 30 min). The supernatants were mixed with 25 μl Nickel-resin pre-equilibrated with equilibration buffer (20 mM HEPES, 500 mM NaCl, 10% glycerol, 10 mM imidazole, 0.5 mM TCEP, pH 7.5) in a 96-well plate with filter units and incubated at room temperature with slow shaking for 30 min. Non-bound protein was then collected by centrifugation (200×g, 1 min) at 4° C. The resin was washed with three times with wash buffer (20 mM HEPES, 500 mM NaCl, 10% glycerol, 30 mM imidazole, 0.5 mM TCEP, pH 7.5) and resuspended in 40 μl elution buffer (20 mM HEPES, 500 mM NaCl, 10% glycerol, 500 mM imidazole, 0.5 mM TCEP, pH 7.5) and incubated at room temperature. The elution fractions were collected by centrifugation. Aliquots of the total cell lysates (FIG. 12A), clarified cell lysates (FIG. 12B) and elution fractions (FIG. 12C) were analyzed by SDS-PAGE, according to standard procedures.

Eluted fractions of wild-type (WT) of KB_A01, KB_A09, and KB_A11 and protein A mutated variants of KB_A01, KB_A09, and KB_A11, denoted KB_A12, KB_A13 and KB_14 herein, along with the positive control 1 were mixed with 25 μl MabSelect PrismA™ resin pre-equilibrated with binding buffer (PBS; 10 mM phosphate, 140 mM NaCl, 2.7 mM KCl, pH 7.4) in a 96-well plate with filter units and incubated at room temperature with slow shaking for 45 min. Unbound protein was collected by centrifugation (200× g, 1 min) at 4° C. The resin was washed with 2×250 μl wash buffer (PBS pH 7.4). To elute the bound protein, the resin was resuspended in 40 μl elution buffer (100 mM sodium citrate pH 3.0), neutralized in 1 M Tris-HCl pH 9.0 and incubated at room temperature. The elution fractions were collected by centrifugation. Aliquots of the flow-through and elution fractions (FIGS. 12B and 12C) were analyzed by SDS-PAGE.

The ability of WT KB_A01, KB_A09 and KB_A11 and the corresponding protein A enabling mutated variants KB_A12, KB_A13 and KB_A14, respectively, along with the positive control 1, to bind target hTfR1 was assessed at a single concentration by SPR analysis using a Biacore T200 instrument. Briefly, hTfR1 was covalently immobilized to the surface of a CM5 chip (Cytiva) by amine coupling using standard reagents. Human TfR1 was diluted to 25 μg/mL in 10 mM sodium acetate pH 5.5 and immobilized for 600 s leading to a very high signal level of about 2300 response units (RUs). Subsequently, all six VHHs were diluted in running buffer (PBS supplemented with 0.05% Tween® 20 (PBS-T)) to 100 nM. A signal equal or higher than the corresponding WT was regarded as “binding similar to WT” (see Table 11).

The ability of WT KB_A01, KB_A09 and KB_A11 and the corresponding protein A enabling mutated variants KB_A12, KB_A13 and KB_A14, respectively, along with the positive control 1, to bind to PrismA™ resin was studied in detail by SPR analysis using a Biacore T200 instrument. Each construct was diluted in running buffer (PBS-T, pH 7.4) to 500 nM and subsequently diluted serially 1:4 four times (500, 125, 31.2, 7.81 and 1.95 nM) and run over a PrismA™ chip (Cytiva). Contact time for each concentration was 30 s at 30 μL/min and dissociation time was 30 s at 30 μL/mim. Surface was regenerated by washing with running buffer for 300 s at 30 μL/min.

Ni-NTA based purification of wildtype KB_A01, KB_A09, and KB_A11 and the MabSelect PrismA™ resin based purification of the protein A enabling mutated variants and the positive control 1 resulted in very pure products in the eluted fractions (see FIG. 12B for representative SDS-PAGE of the PrismA™ eluates for KB_A01, KB_A12 and positive control 1). The majority of the WT KB_A01, KB_A09, and KB_A11 was found in the flow-through of the PrismA™ based purification (see FIG. 12C for SDS-PAGE of the flow-through fractions for KB_A01, KB_A12 and positive control 1) indicating that the mutations in positions 19 and 84 for KB_A01 and position 19 for KB_A09 and KB_A11 resulted in VHHs with the ability to be purified using PrismA™ resin.

Binding to hTfR1 was studies by SPR at a single concentration of 100 nM. All three protein A enabling mutated variants bound to hTfR with affinity similar to that of the corresponding WT VHHs. In contrast, the positive control 1 did not bind to the hTfR1 (see Table 11).

Binding to PrismA™ resin was also verified by SPR with a precoated chip (Cytiva Series S Sensor Chip PrismA™). WT KB_A01 (FIG. 12D), KB_A09 and KB_A11 bound very poorly to PrismA™ resin even at the highest concentration tested (500 nM) whereas KB_A12 (FIG. 12E), KB_A13 and KB_A14 as well as the positive control 1 bound with signals reaching >1400 RU at the highest concentration (500 nM) confirming the functionality of VHH-mediated binding to PrismA™ resin (Table 11).

TABLE 11
Binding data verifying functionality of PrismA ™ enabled VHHs

| | | | Binding |
|---|---|---|---|
| | | | to hTfR1 |
| | Mutations | | similar to |
| | vs. corre- | | corre- |
| | sponding | PrismA ™ | sponding |
| VHH | WT VHH | binding | WT VHH |
| KB_A01 | n/a | No | n/a |
| KB_A12 (KB_A01 PrismA ™) | S19R, N84S | Yes | Yes |
| KB_A09 | n/a | No | n/a |
| KB_A13 (KB_A09 PrismA ™) | S19R | Yes | Yes |
| KB_A11 | n/a | No | n/a |
| KB_A14 (KB_A11 PrismA ™) | S19R | Yes | Yes |
| Positive control 1 | | Yes | no binding |

n/a = not applicable (comparison vs self)

### Example XIV—Humanization of VHHs

17 VHH variants (without His tag) of KB_A01, see Table 12, KB_A12, KB_A16-KB_A31, each mutated in one or several amino acids as compared to KB_A01, were produced in CHO cells by ProteoGenix SAS, France. A positive control (positive control 2) known to bind protein A (SEQ ID NO: 84) was also included. The cDNAs coding for the VHHs were chemically synthesized with optimization for expression in CHO cells and subcloned by ProteoGenix and in Proteogenix's proprietary mammalian cells expression vectors. The vectors were transfected in XtenCHO™ cells by XtenCHO™ transfection protocol. In a total volume of 3.5 mL culture medium was collected 8 days after transfection and purified using Mabselect PrismA™ resin (Cytiva). Culture media was clarified by filtration (0.22 μm) and loaded on the pre-equilibrated (PBS pH 7.5) PrismA™ resin. Bound VHH was eluted by pH shift and the eluate was instantly neutralized by addition of 1 M Tris-HCl pH 9.0. Protein concentration was determined by A280 spectrophotometric measurement.

The ability of KB_A01 and the 18 mutated variants of KB_A01 produced in CHO cells, all carrying the two protein A enabling mutations (S19R, N84S, see Example XII), to bind target hTfR1 was assessed at a single concentration by SPR using a Biacore T200 instrument. E. coli produced variants (Example XII), all fused to a His6 tag of KB_A01, KB_A12, KB_A15, KB_A16, KB_A17, KB_A18 and KB_A19 were also included in the same experiment and run under the same conditions. Briefly, hTfR1 was covalently immobilized to the surface of a CM5 chip (Cytiva) by amine coupling using standard reagents. Human TfR1 was diluted to 15 μg/mL in 10 mM sodium acetate pH 5.5 and immobilizing level was aimed to 1000 response units (RUs). Subsequently, all VHHs were diluted in running buffer (PBS supplemented with 0.05% Tween® 20) to 50 nM. A signal similar or higher than the corresponding WT KB_A01 (for E. coli produced material) or KB_A12 (CHO produced) was regarded as “binding” (see Table 12). The reason for the use of KB_A12 as reference for the CHO produced material is that the WT KB_A01 was not produced as a free VHH without a His6 tag since it has no PrismA™ binding capacity. Similarly, KB_A15 was also only tested fused to a His6 tag.

Using an intermediate immobilization of 1000 RU of the hTfR, the 18 mutated VHH variants of KB3_A01 were analyzed for binding in a screening mode at 50 nM. KB3_A01 and KB3_A15 were only tested as His-tagged proteins produced in E. coli, while KB_A12 KB_A15 KB_A17 KB_A18 and KB_A19 were tested as both His-tagged proteins produced in E. coli and as free VHHs produced in CHO cells. KB_A20-KB_A31 were only produced in CHO cells and purified by PrismA™ resin. As can be seen in Table 12, mutations in position 1, 5, 11, 14, 19, 43, 44, 45, 46, 74, 78, 84, 86, and 109 did not by themselves and/or in combination with some of the above, disturb binding to hTfR1 when analyzed as a single concentration assessment at 50 nM. In contrast, mutations in positions 37, 49, 50, 96 and 97 on top of the ten positions already mutated in KB3_A18 totally disrupted binding to the hTfR1. Mutation in position 47 resulted in a dramatic loss in binding while mutations in positions 58 and 87 resulted in a milder loss of affinity.

TABLE 12
Binding data of humanized VHHs

| Variant | Q1 | Q5 | S11 | A14 | S19 | F37 | E43 | Q44 | R45 | D46 | V47 | A49 | T50 |
|---|---|---|---|---|---|---|---|---|---|---|---|---|---|
| KB_A01 | | | | | | | | | | | | | |
| KB_A12 | | | | | R | | | | | | | | |
| KB_A15 | | | | | R | | | | | | | | |
| KB_A16 | E | V | | P | R | | K | | | | | | |
| KB_A17 | E | V | L | P | R | | K | | | | | | |
| KB_A18 | E | V | L | P | R | | K | | | | | | |
| KB_A19 | E | V | L | P | R | V | K | G | L | E | W | S | A |
| KB_A20 | E | V | L | P | R | V | K | | | | | | |
| KB_A21 | E | V | L | P | R | | K | G | | | | | |
| KB_A22 | E | V | L | P | R | | K | | L | | | | |
| KB_A23 | E | V | L | P | R | | K | | | E | | | |
| KB_A24 | E | V | L | P | R | | K | | | | W | | |
| KB_A25 | E | V | L | P | R | | K | | | | | S | |
| KB_A26 | E | V | L | P | R | | K | | | | | | A |
| KB_A27 | E | V | L | P | R | | K | | | | | | |
| KB_A28 | E | V | L | P | R | | K | | | | | | |
| KB_A29 | E | | | | R | | K | | | | | | |
| KB_A30 | E | V | L | P | R | | K | | | | | | |
| KB_A31 | E | V | L | P | R | | K | | | | | | |

| Variant | E58 | A74 | V78 | N84 | K86 | P87 | W96 | M97 | Q109 | Binding |
|---|---|---|---|---|---|---|---|---|---|---|
| KB_A01 | | | | | | | | | | X |
| KB_A12 | | | | S | | | | | | X |
| KB_A15 | | | I | S | | | | | | X |
| KB_A16 | | | | S | R | | | | | X |
| KB_A17 | | | | S | R | | | | | X |
| KB_A18 | | | I | S | R | | | | L | X |
| KB_A19 | Y | S | L | S | R | A | A | R | L | — |
| KB_A20 | | | I | S | R | | | | L | — |
| KB_A21 | | | I | S | R | | | | L | X |
| KB_A22 | | | I | S | R | | | | L | X |
| KB_A23 | | | I | S | R | | | | L | X |
| KB_A24 | | | I | S | R | | | | L | (—) |
| KB_A25 | | | I | S | R | | | | L | — |
| KB_A26 | | | I | S | R | | | | L | — |
| KB_A27 | Y | | I | S | R | | | | L | (X) |
| KB_A28 | | S | I | S | R | | | | L | X |
| KB_A29 | | | I | S | R | A | | | L | (X) |
| KB_A30 | | | I | S | R | | A | | L | — |
| KB_A31 | | | I | S | R | | | R | L | — |

X = binding similar to control, (X) = binding affinity decreased, (—) = binding affinity severely decreased, — = no apparent binding.

TABLE 13
Sequence information

| | | SEQ |
|---|---|---|
| Denotation | Sequence | ID NO: |
| KB_A01 | GSIFGSKR | 1 |
| CDR1 | | |
| KB_ref | GSIFGFNA | 2 |
| CDR1 | | |
| KB_A01 | ITYRGTT | 3 |
| CDR2 | | |
| KB_ref | IAVAGST | 4 |
| CDR2 | | |
| KB_A01 | WMFTTDNY | 5 |
| and | | |
| KB_A10 | | |
| CDR3 | | |
| KB_ref | WMYATANY | 6 |
| CDR3 | | |
| CDR1 | X₁X₂IX₃GSKR | 7 |
| consensus | | |
| 1 | | |
| CDR2 | ITX₄X₅GTT | 8 |
| consensus | | |
| 1 | | |
| CDR3 | WMFTTX&NY | 9 |
| consensus | | |
| KB_A09 | WMFTTTNY | 10 |
| CDR3 | | |
| CDR1 | GDIX₃GSKR | 11 |
| consensus | | |
| 2 | | |
| CDR2 | ITVX₅GTT | 12 |
| consensus | | |
| 2 | | |
| KB_A09 | GDINGSKR | 13 |
| CDR1 | | |
| KB_A09 | ITVRGTT | 14 |
| CDR2 | | |
| KB_A11 | GDIFGSKR | 15 |
| CDR1 | | |
| KB_A11 | ITVGGTT | 16 |
| CDR2 | | |
| KB_A10 | EIINFGSKR | 17 |
| CDR1 | | |
| KB_A10 | ITYHGTT | 18 |
| CDR2 | | |
| FR1 | QVQLQESGGGX₇VQAGGSLX₈LSCAAS | 19 |
| FR2 | MGWFRQAPGX₉X₁₀RDX₁₁VAT | 20 |
| FR3 | X₁₂YX₁₃DSVKGRFTISRDNAX₁₄NTVYLQMNX₁₅LKPEDTAX₁₆YYC | 21 |
| FR4 | WGQGTQVTVSS | 22 |
| KB_A01 | QVQLQESGGGSVQAGGSLSLSCAASGSIFGSKRMGWFRQAPGEQRDVVATITYRGT | 23 |
| | TEYADSVKGRFTISRDNAKNTVYLQMNNLKPEDTAVYYCWMFTTDNYWGQGTQVTV | |
| | SS | |
| KB_A03 | QVQLQESGGGSVQAGGSLSLSCAASGSIFGSKRMGWFRQAPGEQRDVVATIAVAGS | 24 |
| | TEYADSVKGRFTISRDNAKNTVYLQMNNLKPEDTAVYYCWMFTTDNYWGQGTQVTV | |
| | SS | |
| KB_A04 | QVQLQESGGGSVQAGGSLSLSCAASGSIFGSKRMGWFRQAPGEQRDVVATITYRGT | 25 |
| | TEYADSVKGRFTISRDNAKNTVYLQMNNLKPEDTAVYYCWMYATANYWGQGTQVTV | |
| | SS | |
| KB_A07 | QVQLQESGGGSVQAGGSLSLSCAASGSIFGSKRMGWFRQAPGEQRDVVATIAVAGS | 26 |
| | TEYADSVKGRFTISRDNAKNTVYLQMNNLKPEDTAVYYCWMYATANYWGQGTQVTV | |
| | SS | |
| KB_A09 | QVQLQESGGGLVQAGGSLSLSCAASGDINGSKRMGWFRQAPGKARDVVATITVRGT | 27 |
| | TEYEDSVKGRFTISRDNAKNTVYLQMNSLKPEDTAVYYCWMFTTTNYWGQGTQVTV | |
| | SS | |
| KB_A10 | QVQLQESGGGSVQAGGSLRLSCAASEIIFGSKRMGWFRQAPGKQRDLVATITYHGT | 28 |
| | TKYADSVKGRFTISRDNANNTVYLQMNNLKPEDTAFYYCWMFTTDNYWGQGTQVTV | |
| | SS | |
| KB_A11 | QVQLQESGGGLVQAGGSLSLSCAASGDIFGSKRMGWFRQAPGQQRDVVATITVGGT | 29 |
| | TEYADSVKGRFTISRDNAKNTVYLQMNSLKPEDTAVYYCWMFTTNNYWGQGTQVTV | |
| | SS | |
| His-tagged | AHHHHHHSGRLYWDDLKRKLSEKLDSTDFTGTIKLLNENSYVPREAGSQKDENLAL | 30 |
| ectodomain | YVENQFREFKLSKVWRDQHFVKIQVKDSAQNSVIIVDKNGRLVYLVENPGGYVAYS | |
| of hTfR1 | KAATVTGKLVHANFGTKKDFEDLYTPVNGSIVIVRAGKITFAEKVANAESLNAIGV | |
| | LIYMDQTKFPIVNAELSFFGHAHLGTGDPYTPGFPSENHTQFPPSRSSGLPNIPVQ | |
| | TISRAAAEKLFGNMEGDCPSDWKTDSTCRMVTSESKNVKLTVSNVLKEIKILNIFG | |
| | VIKGFVEPDHYVVVGAQRDAWGPGAAKSGVGTALLLKLAQMFSDMVLKDGFQPSRS | |
| | IIFASWSAGDFGSVGATEWLEGYLSSLHLKAFTYINLDKAVLGTSNFKVSASPLLY | |
| | TLIEKTMQNVKHPVTGQFLYQDSNWASKVEKLTLDNAAFPFLAYSGIPAVSFCFCE | |
| | DTDYPYLGTTMDTYKELIERIPELNKVARAAAEVAGQFVIKLTHDVELNLDYERYN | |
| | SQLLSFVRDLNQYRADIKEMGLSLQWLYSARGDFFRATSRLTTDFGNAEKTDRFVM | |
| | KKLNDRVMRVEYHFLSPYVSPKESPFRHVFWGSGSHTLPALLENLKLRKQNNGAEN | |
| | ETLFRNQLALATWTIQGAANALSGDVWDIDNEF | |
| His-tagged | AHHHHHHGSRLYWADLKTLLSEKLNSIEFADTIKQLSQNTYTPREAGSQKDESLAY | 31 |
| ectodomain | YIENQFHEFKFSKVWRDEHYVKIQVKSSIGQNMVTIVQSNGNLDPVESPEGYVAFS | |
| of mTfR1 | KPTEVSGKLVHANFGTKKDFEELSYSVNGSLVIVRAGEITFAEKVANAQSFNAIGV | |
| | LIYMDKNKFPVVEADLALFGHAHLGTGDPYTPGFPSFNHTQFPPSQSSGLPNIPVQ | |
| | TISRAAAEKLFGKMEGSCPARWNIDSSCKLELSQNQNVKLIVKNVLKERRILNIFG | |
| | VIKGYEEPDRYVVVGAQRDALGAGVAAKSSVGTGLLLKLAQVFSDMISKDGERPSR | |
| | SIIFASWTAGDFGAVGATEWLEGYLSSLHLKAFTYINLDKVVLGTSNFKVSASPLL | |
| | YTLMGKIMQDVKHPVDGKSLYRDSNWISKVEKLSFDNAAYPFLAYSGIPAVSFCFC | |
| | EDADYPYLGTRLDTYEALTQKVPQLNQMVRTAAEVAGQLIIKLTHDVELNLDYEMY | |
| | NSKLLSFMKDLNQFKTDIRDMGLSLQWLYSARGDYFRATSRLTTDFHNAEKTNRFV | |
| | MREINDRIMKVEYHELSPYVSPRESPFRHIFWGSGSHTLSALVENLKLRQKNITAF | |
| | NETLFRNQLALATWTIQGVANALSGDIWNIDNEF | |
| His-tagged | AHHHHHHSGRLYWDDLKRKLSEKLDTTDFTSTIKLLNENLYVPREAGSQKDENLAL | 32 |
| ectodomain | YIENQFREFKLSKVWRDQHFVKIQVKDSAQNSVIIVDKNGGLVYLVENPGGYVAYS | |
| of cTfR1 | KAATVTGKLVHANFGTKKDFEDLDSPVNGSIVIVRAGKITFAEKVANAESLNAIGV | |
| | LIYMDQTKFPIVKADLSFFGHAHLGTGDPYTPGFPSFNHTQFPPSQSSGLPNIPVQ | |
| | TISRAAAEKLFGNMEGDCPSDWKTDSTCKMVTSENKSVKLTVSNVLKETKILNIFG | |
| | VIKGFVEPDHYVVVGAQRDAWGPGAAKSSVGTALLLKLAQMFSDMVLKDGFQPSRS | |
| | IIFASWSAGDFGSVGATEWLEGYLSSLHLKAFTYINLDKAVLGTSNFKVSASPLLY | |
| | TLIEKTMQDVKHPVTGRSLYQDSNWASKVEKLTLDNAAFPFLAYSGIPAVSFCFCE | |
| | DTDYPYLGTTMDTYKELVERIPELNKVARAAAEVAGQFVIKLTHDTELNLDYERYN | |
| | SQLLLFLRDLNQYRADVKEMGLSLQWLYSARGDFFRATSRLTTDERNAEKRDKFVM | |
| | KKLNDRVMRVEYYFLSPYVSPKESPFRHVFWGSGSHTLSALLESLKLRRQNNSAEN | |
| | ETLFRNQLALATWTIQGAANALSGDVWDIDNEF | |
| N-terminal | MDWLRNLLFLMAAAQSINA | 33 |
| signal | | |
| peptide | | |
| CALL001 | GTCCTGGCTGCTCTTCTACAAGG | 34 |
| CALL002 | GGTACGTGCTGTTGAACTGTTCC | 35 |
| VHH-R-SfiI | AAAGGCCCAGCCGGCCATGGCGCAGGTGCAGCTGCAGGAGTCTGGRGGAGG | 36 |
| VHH-F-SfiI | AAAGGCCTCCCGGGCCACGTTTTGAGGAGACGGTGACCTGGGT | 37 |
| 6xHis | HHHHHH | 38 |
| C-tag | EPEA | 39 |
| Human | DKTHTCPPCPAPELLGGPSVFLFPPKPKDTLMISRTPEVTCVVVDVSHEDPEVKEN | 40 |
| IgG1 Fc | WYVDGVEVHNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWESNGQPE | |
| | NNYKTTPPVLDSDGSFFLYSKLTVDKSRWQQGNVFSCSVMHEALHNHYTQKSLSLS | |
| | PGK | |
| KB_A01 Fc | QVQLQESGGGSVQAGGSLSLSCAASGSIFGSKRMGWFRQAPGEQRDVVATITYRGT | 41 |
| fusion | TEYADSVKGRFTISRDNAKNTVYLQMNNLKPEDTAVYYCWMFTTDNYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A02 | QVQLQESGGGSVQAGGSLSLSCAASGSIFGFNAMGWFRQAPGEQRDVVATITYRGT | 42 |
| | TEYADSVKGRFTISRDNAKNTVYLQMNNLKPEDTAVYYCWMFTTDNYWGQGTQVTV | |
| | SS | |
| KB_A05 | QVQLQESGGGSVQAGGSLSLSCAASGSIFGFNAMGWFRQAPGEQRDVVATIAVAGS | 43 |
| | TEYADSVKGRFTISRDNAKNTVYLQMNNLKPEDTAVYYCWMFTTDNYWGQGTQVTV | |
| | SS | |
| KB_A06 | QVQLQESGGGSVQAGGSLSLSCAASGSIFGFNAMGWFRQAPGEQRDVVATITYRGT | 44 |
| | TEYADSVKGRFTISRDNAKNTVYLQMNNLKPEDTAVYYCWMYATANYWGQGTQVTV | |
| | SS | |
| KB_A08 | QVQLQESGGGSVQAGGSLSLSCAASGSIFGFNAMGWFRQAPGEQRDVVATIAVAGS | 45 |
| | TEYADSVKGRFTISRDNAKNTVYLQMNNLKPEDTAVYYCWMYATANYWGQGTQVTV | |
| | SS | |
| 3D6 VH | EVKLVESGGGLVKPGASLKLSCAASGFTFSNYGMSWVRQNSDKRLEWVASIRSGGG | 46 |
| | RTYYSDNVKGRFTISRENAKNTLYLQMSSLKSEDTALYYCVRYDHYSGSSDYWGQG | |
| | TTVTVS | |
| 3D6 VL | YVVMTQTPLTLSVTIGQPASISCKSSQSLLDSDGKTYLNWLLQRPGQSPKRLIYLV | 47 |
| | SKLDSGVPDRFTGSGSGTDFTLKISRIEAEDLGLYYCWQGTHFPRTFGGGTKLEIK | |
| 3x(G₄S) | GGGGSGGGGSGGGGS | 48 |
| linker | | |
| KB_A01- | QVQLQESGGGSVQAGGSLSLSCAASGSIFGSKRMGWFRQAPGEQRDVVATITYRGT | 49 |
| scFv | TEYADSVKGRFTISRDNAKNTVYLQMNNLKPEDTAVYYCWMFTTDNYWGQGTQVTV | |
| | SSGGGGSGGGGSGGGGSEVKLVESGGGLVKPGASLKLSCAASGFTFSNYGMSWVRQ | |
| | NSDKRLEWVASIRSGGGRTYYSDNVKGRFTISRENAKNTLYLQMSSLKSEDTALYY | |
| | CVRYDHYSGSSDYWGQGTTVTVSGGGGSGGGGGGGGSYVVMTQTPLTLSVTIGQP | |
| | ASISCKSSQSLLDSDGKTYLNWLLQRPGQSPKRLIYLVSKLDSGVPDRFTGSGSGT | |
| | DFTLKISRIEAEDLGLYYCWQGTHFPRTFGGGTKLEIKGSENLYFQSHHHHHH | |
| scFv- | EVKLVESGGGLVKPGASLKLSCAASGFTFSNYGMSWVRQNSDKRLEWVASIRSGGG | 50 |
| KB_A01 | RTYYSDNVKGRFTISRENAKNTLYLQMSSLKSEDTALYYCVRYDHYSGSSDYWGQG | |
| | TTVTVSGGGGSGGGGSGGGGSYVVMTQTPLTLSVTIGQPASISCKSSQSLLDSDGK | |
| | TYLNWLLQRPGQSPKRLIYLVSKLDSGVPDRFTGSGSGTDFTLKISRIEAEDLGLY | |
| | YCWQGTHFPRTFGGGTKLEIKGGGGSGGGGSGGGGSQVQLQESGGGSVQAGGSLSL | |
| | SCAASGSIFGSKRMGWFRQAPGEQRDVVATITYRGTTEYADSVKGRFTISRDNAKN | |
| | TVYLQMNNLKPEDTAVYYCWMFTTDNYWGQGTQVTVSSGSENLYFQSHHHHHH | |
| BA1 | EVQLVQSGAEVKKPGESLKISCKGSGYSFTNYWLGWVRQMPGKGLEWMGDIYPGGD | 51 |
| | YPTYSEKFKVQVTISADKSISTAYLQWSSLKASDTAMYYCARSGNYDEVAYWGQGT | |
| | LVTVSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPEVKENWYV | |
| | DGVEVHNAKTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWESNGQPENNY | |
| | KTTPPVLDSDGSFFLYSKLTVDKSRWQQGNVFSCSVMHEALHNHYTQKSLSLSPGK | |
| | DIVMTQTPLSLSVTPGQPASISCRSSQSLVHSNGNTYLHWYLQKPGQSPQLLIYKV | |
| | SNRFSGVPDRFSGSGSGTDFTLKISRVEAEDVGVYYCSQSTHVPWTFGQGTKVEIK | |
| | RTVAAPSVFIFPPSDEQLKSGTASVVCLLNNFYPREAKVQWKVDNALQSGNSQESV | |
| | TEQDSKDSTYSLSSTLTLSKADYEKHKVYACEVTHQGLSSPVTKSENRGEC | |
| BA2 | EVQLQQSGPELVKPGASMKISCKASGYSFTGYTMNWVKQSHGENLEWIGRINPHNG | 52 |
| | GTDYNQKFKDKAPLTVDKSSNTAYMELLSLTSEDSAVYYCARGYYYYSLDYWGQGT | |
| | SVTVSSASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEPKSCDKT | |
| | HTCPPCPAPELLGGPSVFLFPPKPKDTLMISRTPEVTCVVVDVSHEDPEVKENWYV | |
| | DGVEVHNAKTKPREEQYNSTYRVVSVLTVLHQDWLNGKEYKCKVSNKALPAPIEKT | |
| | ISKAKGQPREPQVYTLPPSRDELTKNQVSLTCLVKGFYPSDIAVEWESNGQPENNY | |
| | KTTPPVLDSDGSFFLYSKLTVDKSRWQQGNVFSCSVMHEALHNHYTQKSLSLSPGK | |
| | QIVLTQSPAIMSASPGEKVTMTCSASSSIDYIHWYQQKSGTSPKRWIYDTSKLASG | |
| | VPARFSGSGSGTSYSLTISSMEPEDAATYYCHQRNSYPWTFGGGTRLEIRRTVAAP | |
| | SVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSENRGEC | |
| KB_ref | QVQLQESGGGLVQAGGSLRLSCAASGSIFGFNAMGWFRQAPGKERDLVATIAVAGS | 53 |
| | TEYADSVKGRFTISRDNAKNTMYLQMNSLKPEDTAVYYCWMYATANYWGQGTQVTV | |
| | SS | |
| Apical | ACCGGTTCTAGCGGCAGCCCTAACATCCCCGTGCAGACCATCAGCAGAGCCGCCGC | 54 |
| hTfR1 | TGAAAAGCTGTTCGGCAACATGGAAGGCGACTGCCCTAGCGATTGGAAGACCGACT | |
| domain | CTACATGTAGAATGGTGACCAGCGAGAGCAAGAATGTGAAGCTCACAGTGTCCAAC | |
| | GTGGGCGGCGGCCAGGTGAAAGACAGCGCCCAGAACAGCGTGATTATCGTTGATAA | |
| | GAACGGCAGACTGGTGTACCTGGTGGAAAACCCCGGCGGCTACGTGGCCAGCTCTA | |
| | AGGCCGCTACAGTGACCGGCAAGCTGGTCCACGCCAATTTCGGCACCAAAAAGGAT | |
| | TTCGAGGACCTGTACACCCCTGTGAACGGCTCCATCGTGATCGTGCGGGCCGGCAA | |
| | AATCACCTTCGCCGAGAAGGTGGCTAATGCCGAGAGCCTGAACGCCATCGGCGTGC | |
| | TGATCTATATGGACCAAACAAAGTTCCCAATCGTCAACGCCGAGCTGTCTTTTGGC | |
| | GGAGCTGGAGGAGCCGGAGGTGCGGCCGC | |
| KB_A11 | WMFTTNNY | 55 |
| CDR3 | | |
| Mouse | MGWSCIILFLVATATGVHS | 56 |
| heavy chain | | |
| signal | | |
| peptide | | |
| KB_A01 | QVQLQESGGGSVQAGGSLSLSCAAS | 57 |
| FR1 | | |
| KB_A01 | MGWFRQAPGEQRDVVAT | 58 |
| FR2 | | |
| KB_A01 | EYADSVKGRFTISRDNAKNTVYLQMNNLKPEDTAVYYC | 59 |
| FR3 | | |
| VHH- | EVQLVESGGGVVQPGGSLKLSCVASGTDFSINFIRWYRQAPGKQREFVAGFTATGN | 60 |
| antibody in | TNYADSMKGRFTISRDNTKNAVYLQIDSLKPEDTAVYYCYMLDKWGQGTQVTVSS | |
| BV | | |
| murine lgG | METDTLLLWVLLLWVPGST | 61 |
| kappa light | | |
| chain signal | | |
| peptide | | |
| KB_A02 Fc | QVQLQESGGGSVQAGGSLSLSCAASGSIFGFNAMGWFRQAPGEQRDVVATITYRGT | 62 |
| fusion | TEYADSVKGRFTISRDNAKNTVYLQMNNLKPEDTAVYYCWMFTTDNYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A03 Fc | QVQLQESGGGSVQAGGSLSLSCAASGSIFGSKRMGWFRQAPGEQRDVVATIAVAGS | 63 |
| fusion | TEYADSVKGRFTISRDNAKNTVYLQMNNLKPEDTAVYYCWMFTTDNYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A04 Fc | QVQLQESGGGSVQAGGSLSLSCAASGSIFGSKRMGWFRQAPGEQRDVVATITYRGT | 64 |
| fusion | TEYADSVKGRFTISRDNAKNTVYLQMNNLKPEDTAVYYCWMYATANYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A05 Fc | QVQLQESGGGSVQAGGSLSLSCAASGSIFGFNAMGWFRQAPGEQRDVVATIAVAGS | 65 |
| fusion | TEYADSVKGRFTISRDNAKNTVYLQMNNLKPEDTAVYYCWMFTTDNYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A06 Fc | QVQLQESGGGSVQAGGSLSLSCAASGSIFGFNAMGWFRQAPGEQRDVVATITYRGT | 66 |
| fusion | TEYADSVKGRFTISRDNAKNTVYLQMNNLKPEDTAVYYCWMYATANYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A07 Fc | QVQLQESGGGSVQAGGSLSLSCAASGSIFGSKRMGWFRQAPGEQRDVVATIAVAGT | 67 |
| fusion | TEYADSVKGRFTISRDNAKNTVYLQMNNLKPEDTAVYYCWMYATANYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A08 Fc | QVQLQESGGGSVQAGGSLSLSCAASGSIFGFNAMGWFRQAPGEQRDVVATIAVAGS | 68 |
| fusion | TEYADSVKGRFTISRDNAKNTVYLQMNNLKPEDTAVYYCWMYATANYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A09 Fc | QVQLQESGGGLVQAGGSLSLSCAASGDINGSKRMGWFRQAPGKARDVVATITVRGT | 69 |
| fusion | TEYEDSVKGRFTISRDNAKNTVYLQMNSLKPEDTAVYYCWMFTTTNYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A10 Fc | QVQLQESGGGSVQAGGSLRLSCAASEIIFGSKRMGWFRQAPGKQRDLVATITYHGT | 70 |
| fusion | TKYADSVKGRFTISRDNANNTVYLQMNNLKPEDTAFYYCWMFTTDNYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| KB_A11 Fc | QVQLQESGGGLVQAGGSLSLSCAASGDIFGSKRMGWFRQAPGQQRDVVATITVGGT | 71 |
| fusion | TEYADSVKGRFTISRDNAKNTVYLQMNSLKPEDTAVYYCWMFTTNNYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |
| Apical | TGSSGSPNIPVQTISRAAAEKLFGNMEGDCPSDWKTDSTCRMVTSESKNVKLTVSN | 72 |
| domain | VGGGQVKDSAQNSVIIVDKNGRLVYLVENPGGYVASSKAATVTGKLVHANFGTKKD | |
| construct of | FEDLYTPVNGSIVIVRAGKITFAEKVANAESLNAIGVLIYMDQTKEPIVNAELSFG | |
| hTfR1 | GAGGAGGAA | |
| KB_A12 | QVQLQESGGGSVQAGGSLRLSCAASGSIFGSKRMGWFRQAPGEQRDVVATITYRGT | 73 |
| | TEYADSVKGRFTISRDNAKNTVYLQMNSLKPEDTAVYYCWMFTTDNYWGQGTQVTV | |
| | SS | |
| KB_A13 | QVQLQESGGGLVQAGGSLRLSCAASGDINGSKRMGWFRQAPGKARDVVATITVRGT | 74 |
| | TEYEDSVKGRFTISRDNAKNTVYLQMNSLKPEDTAVYYCWMFTTTNYWGQGTQVTV | |
| | SS | |
| KB_A14 | QVQLQESGGGLVQAGGSLRLSCAASGDIFGSKRMGWFRQAPGQQRDVVATITVGGT | 75 |
| | TEYADSVKGRFTISRDNAKNTVYLQMNSLKPEDTAVYYCWMFTTNNYWGQGTQVTV | |
| | SS | |
| ProA | QVQLQESGGGLVQPGGSLRLSCAASGFTFSSYAMSWVRQAPGKGLEWVSAISGSGG | 76 |
| positive | STYYADSVKGRFTISRDNSKNTLYLQMNSLRAEDTAVYYCGTDMEVWGKGTTVTVS | |
| control 1 | S | |
| His6-tag | GGHHHHHH | 77 |
| FR1 | X₁VQLX₂ESGGGX₃VQX₄GGSLX₅LSCAAS | 78 |
| consensus | | |
| FR2 | MGWFRQAPGX₆X₇X₈X₉VVAT | 79 |
| consensus | | |
| FR3 | EYADSVKGRFTISRDNX₁₀KNTX₁₁YLQMNX₁₂LX₁₃PEDTAVYYC | 80 |
| consensus | | |
| FR4 | WGQGTX₁₄VTVSS | 81 |
| consensus | | |
| FR1 protein | QVQLQESGGGSVQAGGSLRLSCAAS | 82 |
| A | | |
| FR3 protein | EYADSVKGRFTISRDNAKNTVYLQMNSLKPEDTAVYYC | 83 |
| A | | |
| ProA | QVQLQESGGGLVQPGGSLRLSCAASGRTFSSYAMGWFRQAPGKQREFVAAIRWSGG | 84 |
| positive | YTYYTDSVKGRFTISRDNAKTTVYLQMNSLKPEDTAVYYCAATYLSSDYSRYALPQ | |
| control 2 | RPLDYDYWGQGTQVTVSSLE | |
| KB_ref Fc | QVQLQESGGGLVQAGGSLRLSCAASGSIFGFNAMGWFRQAPGKERDLVATIAVAGS | 85 |
| fusion | TEYADSVKGRFTISRDNAKNTMYLQMNSLKPEDTAVYYCWMYATANYWGQGTQVTV | |
| | SSGGDKTHTCPPCPAPELLGGPSVFLFPPKPKDTLMISRTPEVTCVVVDVSHEDPE | |
| | VKFNWYVDGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDIAVEWESN | |
| | GQPENNYKTTPPVLDSDGSFFLYSKLTVDKSRWQQGNVFSCSVMHEALHNHYTQKS | |
| | LSLSPGK | |

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-38. (canceled)

39. A variable domain of heavy chain-only, VHH, antibody binding specifically to a transferrin receptor 1, TfR1, comprising:
a complementarity determining region 1, CDR1, having an amino acid sequence selected from the group consisting of GSIFGSKR as defined in SEQ ID NO: 1 and GSIFGFNA as defined in SEQ ID NO: 2;
a CDR2 having an amino acid sequence selected from the group consisting of ITYRGTT as defined in SEQ ID NO: 3 and IAVAGST as defined in SEQ ID NO: 4; and
a CDR3 having an amino acid sequence selected from the group consisting of WMFTTDNY as defined in SEQ ID NO: 5 and WMYATANY as defined in SEQ ID NO: 6, wherein
if the CDR1 has the amino acid sequence as defined in SEQ ID NO: 2, then the CDR3 has the amino acid sequence as defined in SEQ ID NO: 6;
with the proviso that the VHH antibody does not comprise a CDR1 having the amino acid sequence as defined in SEQ ID NO: 2, a CDR2 having the amino acid sequence as defined in SEQ ID NO: 4, and a CDR3 having the amino acid sequence as defined in SEQ ID NO: 6.

40. The VHH antibody according to claim 39, with the proviso that the VHH antibody does not comprise a CDR1 having the amino acid sequence as defined in SEQ ID NO: 2, a CDR2 having the amino acid sequence SEQ ID NO: 3, and a CDR3 having the amino acid sequence as defined in SEQ ID NO: 6.

41. The VHH antibody according to claim 39, wherein
the CDR1 has the amino acid sequence as defined in SEQ ID NO: 1;
the CDR2 has the amino acid sequence selected from the group consisting of SEQ ID NO: 3 and 4; and
the CDR3 has the amino acid sequence as defined in SEQ ID NO: 5.

42. The VHH antibody according to claim 41, wherein
the CDR1 has the amino acid sequence as defined in SEQ ID NO: 1;
the CDR2 has the amino acid sequence as defined in SEQ ID NO: 4; and
the CDR3 has the amino acid sequence as defined in SEQ ID NO: 5.

43. The VHH antibody according to claim 41, wherein
the CDR1 has the amino acid sequence as defined in SEQ ID NO: 1;
the CDR2 has the amino acid sequence as defined in SEQ ID NO: 3; and
the CDR3 has the amino acid sequence as defined in SEQ ID NO: 5.

44. The VHH antibody according to claim 39, wherein
the CDR1 has the amino acid sequence selected from the group consisting of SEQ ID NO: 1 and 2;
the CDR2 has the amino acid sequence as defined in SEQ ID NO: 3; and
the CDR3 has the amino acid sequence as defined in SEQ ID NO: 6.

45. The VHH antibody according to claim 44, wherein
the CDR1 has the amino acid sequence as defined in SEQ ID NO: 1;
the CDR2 has the amino acid sequence as defined in SEQ ID NO: 3; and
the CDR3 has the amino acid sequence as defined in SEQ ID NO: 6.

46. The VHH antibody according to claim 39, wherein
the CDR1 has the amino acid sequence as defined in SEQ ID NO: 1;
the CDR2 has the amino acid sequence as defined in SEQ ID NO: 4; and
the CDR3 has the amino acid sequence as defined in SEQ ID NO: 6.

47. The VHH antibody according to claim 39, wherein
the VHH antibody is of formula: framework region 1, FR1, -CDR1-FR2-CDR2-FR3-CDR3-FR4;
FR1 has an amino acid sequence QVQLQESGGGSVQAGGSLSLSCAAS as defined in SEQ ID NO: 57;
FR2 has an amino acid sequence MGWFRQAPGEQRDVVAT as defined in SEQ ID NO: 58;
FR3 has an amino acid sequence EYADSVKGRFTISRDNAKNTVYLQMNNLKPEDTAVYYC as defined in SEQ ID NO: 59; and
FR4 has an amino acid sequence WGQGTQVTVSS as defined in SEQ ID NO: 22.

48. The VHH antibody according to claim 39, wherein the VHH antibody has an amino acid sequence selected from the group consisting of SEQ ID NO: 23 to 26.

49. A variable domain of heavy chain-only, VHH, antibody binding specifically to a transferrin receptor 1, TfR1, comprising:
a complementarity determining region 1, CDR1, consisting of the amino acid sequence X₁X₂IX₃GSKR as defined in SEQ ID NO: 7, wherein X₁ is G or E, X₂ is S, D or I, and X₃ is F or N;
a CDR2 consisting of the amino acid sequence ITX₄X₅GTT as defined in SEQ ID NO: 8, wherein X₄ is Y or V, and X₅ is R, H or G; and
a CDR3 consisting of the amino acid sequence WMFTTX₆NY as defined in SEQ ID NO: 9, wherein X₆ is D, T or N.

50. The VHH antibody according to claim 49, wherein
the CDR1 consists of the amino acid sequence X₁X₂IX₃GSKR as defined in SEQ ID NO: 7, wherein X₁ is G or E, X₂ is D or I, and X₃ is F or N;
the CDR2 consists of the amino acid sequence ITX₄X₅GTT as defined in SEQ ID NO: 8, wherein X₄ is Y or V, and X₅ is R, H or G; and
the CDR3 consists of the amino acid sequence WMFTTX₆NY as defined in SEQ ID NO: 9, wherein X₆ is 1, T or N.

51. The VHH antibody according to claim 50, wherein
the CDR11 consists of the amino acid sequence GDIX₃GSKR as defined in SEQ ID NO: 11, wherein X₃ is F or N;
the CDR2 consists of the amino acid sequence ITVX₅GTT as defined in SEQ ID NO: 12, wherein X₅ is R or G; and
the CDR3 consists of the amino acid sequence WMFTTX₆NY as defined in SEQ ID NO: 9, wherein X₆ is T or N.

52. The VHH antibody according to claim 51, wherein
the CDR11 consists of the amino acid sequence GDINGSKR as defined in SEQ ID NO: 13;
the CDR2 consists of the amino acid sequence ITVRGTT as defined in SEQ ID NO: 14; and
the CDR3 consists of the amino acid sequence WMFTTTNY as defined in SEQ ID NO: 10.

53. The VHH antibody according to claim 51, wherein
the CDR11 consists of the amino acid sequence GDIFGSKR as defined in SEQ ID NO: 15;
the CDR2 consists of the amino acid sequence ITVGGTT as defined in SEQ ID NO: 16; and
the CDR3 consists of the amino acid sequence WMFTTNNY as defined in SEQ ID NO: 54.

54. The VHH antibody according to claim 50, wherein
the CDR1 consists of the amino acid sequence EIINFGSKR as defined in SEQ ID NO: 17;
the CDR2 consists of the amino acid sequence ITYHGTT as defined in SEQ ID NO: 18; and
the CDR3 consists of the amino acid sequence WMFTTDNY as defined in SEQ ID NO: 5.

55. The VHH antibody according to claim 49, wherein
the VHH antibody is of formula: framework region 1, FR1, -CDR1-FR2-CDR2-FR3-CDR3-FR4;
FR1 has an amino acid sequence QVQLQESGGGX₇VQAGGSLX₈LSCAAS as defined in SEQ ID NO: 19, wherein X₇ is S or L, and X₈ S or R;
FR2 has an amino acid sequence MGWFRQAPGX₉X₁₀RDX₁₁VAT as defined in SEQ ID NO: 20, wherein X₉ is E, K or Q, preferably K or Q, X₁₀ is Q or A, and X₁₁ is V or L;
FR3 has an amino acid sequence X₁₂YX₁₃DSVKGRFTISRNAX₁₄NTVYLQMNX₁₅LKPEDTAX₁₆YYC as defined in SEQ ID NO: 21, wherein X₁₂ is E or K, X₁₃ is A or E, X₁₄ is K or N, X₁₅ is N or S, and X₁₆ is V or F; and
FR4 has an amino acid sequence WGQGTQVTVSS as defined in SEQ ID NO: 22.

56. The VHH antibody according to claim 49, wherein the VHH antibody has an amino acid sequence selected from the group consisting of SEQ ID NO: 23, 27 to 29, preferably selected from the group consisting of SEQ ID NO: 27 to 29.

57. The VHH antibody according to claim 39 or 49, wherein the VHH antibody binds specifically to human TfR1.

58. The VHH antibody according to claim 57, wherein the VHH antibody binds specifically to an apical domain of the human TfR1.

59. The VHH antibody according to claim 57, wherein the VHH antibody binds to the human TfR1 with an affinity K_{D} selected within a range of from 0.1 nM to 150 nM.

60. The VHH antibody according to claim 59, wherein the VHH antibody binds to the human TfR1 with an affinity K_{D} selected within a range of from 0.1 nM and 100 nM.

61. The VHH antibody according to claim 39 or 49, wherein the VHH antibody binds specifically to cynomolgus TfR1.

62. The VHH antibody according to claim 39 or 49, wherein the VHH antibody is a camelid VHH antibody.

63. The VHH antibody according to claim 39 or 49, wherein the VHH antibody is a humanized VHH antibody.

64. The VHH antibody according to claim 63, wherein the VHH antibody comprises:
a framework region 1, FR1, according to X₁VQLX₂ESGGGX₃VQX₄GGSLX₅LSCAAS as defined in SEQ LD NO: 78;
a FR2 according to MGWFRQAPGX₆X₇X₉X₉VVAT FR2 as defined in SEQ ID NO: 79;
a FR3 according to EYADSVKGRFISRDNX₁₀KNTX₁₁YLQMNX₁₂LX₁₃PEDTAVYYC as defined in SEQ ID NO: 80; and
a FR4 according to WGQGTX₁₄VTVSS as defined in SEQ ID NO: 81, wherein
at least one of X₁ to X₁₄ is according to X₁=E, X₂=V, X₃=L, X₄=P, X₅=R, X₆=K, X₇=G, X₈=L, X₉=E, X₁₀=S, X₁₁=I, X₁₂=S, X₁₃=R, and X₁₄=L; and
any remaining one(s) of X₁ to X₁₄ is according to X₁=Q, X₂=Q, X₃=S, X₄=A, X₅=S, X₆=E, X₇=Q, X₈=R, X₉=D, X₁₀=A, X₁₁=V, X₁₂=N, X₁₃=K, and X₁₄=Q.

65. The VHH antibody according to claim 39 or 49, wherein the VHH antibody binds to protein A.

66. The VHH antibody according to claim 65, wherein the VHH antibody has a framework region 1, FR1, according to QVQLQESGGGSVQAGGSLRLSCAAS according to SEQ ID NO: 82 and a FR3 according to EYADSVKGRFTISRDNAKNTVYLQMNSLKPEDTAVYYC according to SEQ ID NO: 83.

67. A fusion molecule comprising a VHH antibody according to claim 39 or 49 linked to at least one molecule.

68. The fusion molecule according to claim 67, wherein the fusion molecule comprises the VHH antibody according to claim 39 or 49 linked to the at least one molecule through a linker.

69. The fusion molecule according to claim 67, wherein the at least one molecule is selected from the group consisting of a therapeutic agent and an imaging agent.

70. The fusion molecule according to claim 69, wherein the therapeutic agent is selected from the group consisting of a therapeutic agent capable of treating a central nervous system disease or disorder, a therapeutic agent is capable of treating cancer, and a therapeutic agent is capable of treating muscular dystrophy.

71. The fusion molecule according to claim 69, wherein the imaging agent is selected from the group consisting of a position emission tomography tracer, a single-photon emission computerized tomography tracer, a fluorescent probe, a luminescent probe, a metal complex containing probe, and a near infrared fluorescent probe.

72. A pharmaceutical composition comprising:
a fusion molecule according to claim 67, wherein the at least one molecule is a therapeutic agent; and
a pharmaceutically acceptable vehicle.

73. A nucleic acid molecule encoding a VHH antibody according to claim 39 or 49 or a fusion molecule according to claim 67.

74. An expression vector comprising a nucleic acid molecule according to claim 73 operably linked to a promoter.

75. A host cell comprising a nucleic acid molecule according to claim 73 or an expression vector according to claim 74.

76. A method of treating a central nervous system disease or disorder in a patient, the method comprises administering an effective amount of a fusion molecule according to claim 70 or a pharmaceutical composition according to claim 72 to the patient, wherein the therapeutic agent is a therapeutic agent capable of treating the central nervous system disease or disorder.

77. A method of treating cancer in a patient, the method comprises administering an effective amount of a fusion molecule according to claim 70 or a pharmaceutical composition according to claim 72 to the patient, wherein the therapeutic agent is a therapeutic agent capable of treating cancer.

78. A method of treating muscular dystrophy in a patient, the method comprises administering an effective amount of a fusion molecule according to claim 70 or a pharmaceutical composition according to claim 72 to the patient, wherein the therapeutic agent is a therapeutic agent capable of treating muscular dystrophy.


SEQUENCE LISTING (85 sequences)

SEQ ID NO: 1 - protein, 8 aa, Lama glama
GSIFGSKR

SEQ ID NO: 2 - protein, 8 aa, Lama glama
GSIFGFNA

SEQ ID NO: 3 - protein, 7 aa, Lama glama
ITYRGTT

SEQ ID NO: 4 - protein, 7 aa, Lama glama
IAVAGST

SEQ ID NO: 5 - protein, 8 aa, Lama glama
WMFTTDNY

SEQ ID NO: 6 - protein, 8 aa, Lama glama
WMYATANY

SEQ ID NO: 7 - protein, 8 aa, synthetic construct
  VARIANT 1: note = G, E
  VARIANT 2: note = S, D, I
  VARIANT 4: note = F, N
XXIXGSKR

SEQ ID NO: 8 - protein, 7 aa, synthetic construct
  VARIANT 3: note = Y, V
  VARIANT 4: note = R, H, G
ITXXGTT

SEQ ID NO: 9 - protein, 8 aa, synthetic construct
  VARIANT 6: note = D, T, N
WMFTTXNY

SEQ ID NO: 10 - protein, 8 aa, Lama glama
WMFTTTNY

SEQ ID NO: 11 - protein, 8 aa, synthetic construct
  VARIANT 4: note = F, N
GDIXGSKR

SEQ ID NO: 12 - protein, 7 aa, synthetic construct
  VARIANT 4: note = R, G
ITVXGTT

SEQ ID NO: 13 - protein, 8 aa, Lama glama
GDINGSKR

SEQ ID NO: 14 - protein, 7 aa, Lama glama
ITVRGTT

SEQ ID NO: 15 - protein, 8 aa, Lama glama
GDIFGSKR

SEQ ID NO: 16 - protein, 7 aa, Lama glama
ITVGGTT

SEQ ID NO: 17 - protein, 9 aa, Lama glama
EIINFGSKR

SEQ ID NO: 18 - protein, 7 aa, Lama glama
ITYHGTT

SEQ ID NO: 19 - protein, 25 aa, synthetic construct
  VARIANT 11: note = S, L
  VARIANT 19: note = S, R
QVQLQESGGGXVQAGGSLXLSCAAS

SEQ ID NO: 20 - protein, 17 aa, synthetic construct
  VARIANT 10: note = E, K, Q
  VARIANT 11: note = Q, A
  VARIANT 14: note = V, L
MGWFRQAPGXXRDXVAT

SEQ ID NO: 21 - protein, 38 aa, synthetic construct
  VARIANT 1: note = E, K
  VARIANT 3: note = A, E
  VARIANT 18: note = K, N
  VARIANT 27: note = N, S
  VARIANT 35: note = V, F
XYXDSVKGRFTISRDNAXNTVYLQMNXLKPEDTAXYYC

SEQ ID NO: 22 - protein, 11 aa, Lama glama
WGQGTQVTVSS

SEQ ID NO: 23 - protein, 114 aa, Lama glama
QVQLQESGGGSVQAGGSLSLSCAASGSIFGSKRMGWFRQAPGEQRDVVATITYRGTTEYADSVKGRFTISRDNAKNTVYLQMNNLKPEDTAVYYCWMFTTDNYWGQGTQVTVSS

SEQ ID NO: 24 - protein, 114 aa, synthetic construct
QVQLQESGGGSVQAGGSLSLSCAASGSIFGSKRMGWFRQAPGEQRDVVATIAVAGSTEYADSVKGRFTISRDNAKNTVYLQMNNLKPEDTAVYYCWMFTTDNYWGQGTQVTVSS

SEQ ID NO: 25 - protein, 114 aa, synthetic construct
QVQLQESGGGSVQAGGSLSLSCAASGSIFGSKRMGWFRQAPGEQRDVVATITYRGTTEYADSVKGRFTISRDNAKNTVYLQMNNLKPEDTAVYYCWMYATANYWGQGTQVTVSS

SEQ ID NO: 26 - protein, 114 aa, synthetic construct
QVQLQESGGGSVQAGGSLSLSCAASGSIFGSKRMGWFRQAPGEQRDVVATIAVAGTTEYADSVKGRFTISRDNAKNTVYLQMNNLKPEDTAVYYCWMYATANYWGQGTQVTVSS

SEQ ID NO: 27 - protein, 114 aa, Lama glama
QVQLQESGGGSVQAGGSLSLSCAASGSIFGSKRMGWFRQAPGEQRDVVATIAVAGSTEYADSVKGRFTISRDNAKNTVYLQMNNLKPEDTAVYYCWMYATANYWGQGTQVTVSS

SEQ ID NO: 28 - protein, 114 aa, Lama glama
QVQLQESGGGSVQAGGSLRLSCAASEIIFGSKRMGWFRQAPGKQRDLVATITYHGTTKYADSVKGRFTISRDNANNTVYLQMNNLKPEDTAFYYCWMFTTDNYWGQGTQVTVSS

SEQ ID NO: 29 - protein, 114 aa, Lama glama
QVQLQESGGGLVQAGGSLSLSCAASGDIFGSKRMGWFRQAPGQQRDVVATITVGGTTEYADSVKGRFTISRDNAKNTVYLQMNSLKPEDTAVYYCWMFTTNNYWGQGTQVTVSS

SEQ ID NO: 30 - protein, 649 aa, Homo sapiens
AHHHHHHSGRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 31 - protein, 650 aa, Mus musculus
AHHHHHHGSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 32 - protein, 649 aa, Macaca fascicularis
AHHHHHHSGRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 33 - protein, 19 aa, synthetic construct
MDWLRNLLFLMAAAQSINA

SEQ ID NO: 34 - DNA, 23 bp, synthetic construct
  source 1..23: mol_type = other DNA
gtcctggctgctcttctacaagg

SEQ ID NO: 35 - DNA, 23 bp, synthetic construct
  source 1..23: mol_type = other DNA
ggtacgtgctgttgaactgttcc

SEQ ID NO: 36 - DNA, 51 bp, synthetic construct
  source 1..51: mol_type = other DNA
aaaggcccagccggccatggcgcaggtgcagctgcaggagtctggrggagg

SEQ ID NO: 37 - DNA, 43 bp, synthetic construct
  source 1..43: mol_type = other DNA
aaaggcctcccgggccacgttttgaggagacggtgacctgggt

SEQ ID NO: 38 - protein, 6 aa, synthetic construct
HHHHHH

SEQ ID NO: 39 - protein, 4 aa, synthetic construct
EPEA

SEQ ID NO: 40 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 343 aa, synthetic construct
QVQLQESGGGSVQAGGSLSLSCAASGSIFGSKRMGWFRQAPGEQRDVVATITYRGTTEYADSVKGRFTISRDNAKNTVYLQMNNLKPEDTAVYYCWMFTTDNYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 114 aa, synthetic construct
QVQLQESGGGSVQAGGSLSLSCAASGSIFGFNAMGWFRQAPGEQRDVVATITYRGTTEYADSVKGRFTISRDNAKNTVYLQMNNLKPEDTAVYYCWMFTTDNYWGQGTQVTVSS

SEQ ID NO: 43 - protein, 114 aa, synthetic construct
QVQLQESGGGSVQAGGSLSLSCAASGSIFGFNAMGWFRQAPGEQRDVVATIAVAGSTEYADSVKGRFTISRDNAKNTVYLQMNNLKPEDTAVYYCWMFTTDNYWGQGTQVTVSS

SEQ ID NO: 44 - protein, 114 aa, synthetic construct
QVQLQESGGGSVQAGGSLSLSCAASGSIFGFNAMGWFRQAPGEQRDVVATITYRGTTEYADSVKGRFTISRDNAKNTVYLQMNNLKPEDTAVYYCWMYATANYWGQGTQVTVSS

SEQ ID NO: 45 - protein, 114 aa, synthetic construct
QVQLQESGGGSVQAGGSLSLSCAASGSIFGFNAMGWFRQAPGEQRDVVATIAVAGSTEYADSVKGRFTISRDNAKNTVYLQMNNLKPEDTAVYYCWMYATANYWGQGTQVTVSS

SEQ ID NO: 46 - protein, 118 aa, synthetic construct
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVS

SEQ ID NO: 47 - protein, 112 aa, synthetic construct
YVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 48 - protein, 15 aa, synthetic construct
GGGGSGGGGSGGGGS

SEQ ID NO: 49 - protein, 389 aa, synthetic construct
QVQLQESGGGSVQAGGSLSLSCAASGSIFGSKRMGWFRQAPGEQRDVVATITYRGTTEYADSVKGRFTISRDNAKNTVYLQMNNLKPEDTAVYYCWMFTTDNYWGQGTQVTVSSGGGGSGGGGSGGGGSEVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGSENLYFQSHHHHHH

SEQ ID NO: 50 - protein, 389 aa, synthetic construct
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSGGGGSGGGGSGGGGSYVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIKGGGGSGGGGSGGGGSQVQLQESGGGSVQAGGSLSLSCAASGSIFGSKRMGWFRQAPGEQRDVVATITYRGTTEYADSVKGRFTISRDNAKNTVYLQMNNLKPEDTAVYYCWMFTTDNYWGQGTQVTVSSGSENLYFQSHHHHHH

SEQ ID NO: 51 - protein, 667 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 661 aa, synthetic construct
EVQLQQSGPELVKPGASMKISCKASGYSFTGYTMNWVKQSHGENLEWIGRINPHNGGTDYNQKFKDKAPLTVDKSSNTAYMELLSLTSEDSAVYYCARGYYYYSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKQIVLTQSPAIMSASPGEKVTMTCSASSSIDYIHWYQQKSGTSPKRWIYDTSKLASGVPARFSGSGSGTSYSLTISSMEPEDAATYYCHQRNSYPWTFGGGTRLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 114 aa, synthetic construct
QVQLQESGGGLVQAGGSLRLSCAASGSIFGFNAMGWFRQAPGKERDLVATIAVAGSTEYADSVKGRFTISRDNAKNTMYLQMNSLKPEDTAVYYCWMYATANYWGQGTQVTVSS

SEQ ID NO: 54 - DNA, 533 bp, Homo sapiens
  source 1..533: mol_type = genomic DNA
accggttctagcggcagccctaacatccccgtgcagaccatcagcagagccgccgctgaaaagctgttcggcaacatggaaggcgactgccctagcgattggaagaccgactctacatgtagaatggtgaccagcgagagcaagaatgtgaagctcacagtgtccaacgtgggcggcggccaggtgaaagacagcgcccagaacagcgtgattatcgttgataagaacggcagactggtgtacctggtggaaaaccccggcggctacgtggccagctctaaggccgctacagtgaccggcaagctggtccacgccaatttcggcaccaaaaaggatttcgaggacctgtacacccctgtgaacggctccatcgtgatcgtgcgggccggcaaaatcaccttcgccgagaaggtggctaatgccgagagcctgaacgccatcggcgtgctgatctatatggaccaaacaaagttcccaatcgtcaacgccgagctgtcttttggcggagctggaggagccggaggtgcggccgc

SEQ ID NO: 55 - protein, 8 aa, Lama glama
WMFTTNNY

SEQ ID NO: 56 - protein, 19 aa, synthetic construct
MGWSCIILFLVATATGVHS

SEQ ID NO: 57 - protein, 25 aa, Lama glama
QVQLQESGGGSVQAGGSLSLSCAAS

SEQ ID NO: 58 - protein, 17 aa, Lama glama
MGWFRQAPGEQRDVVAT

SEQ ID NO: 59 - protein, 38 aa, Lama glama
EYADSVKGRFTISRDNAKNTVYLQMNNLKPEDTAVYYC

SEQ ID NO: 60 - protein, 111 aa, Lama glama
EVQLVESGGGVVQPGGSLKLSCVASGTDFSINFIRWYRQAPGKQREFVAGFTATGNTNYADSMKGRFTISRDNTKNAVYLQIDSLKPEDTAVYYCYMLDKWGQGTQVTVSS

SEQ ID NO: 61 - protein, 19 aa, Mus musculus
METDTLLLWVLLLWVPGST

SEQ ID NO: 62 - protein, 343 aa, synthetic construct
QVQLQESGGGSVQAGGSLSLSCAASGSIFGFNAMGWFRQAPGEQRDVVATITYRGTTEYADSVKGRFTISRDNAKNTVYLQMNNLKPEDTAVYYCWMFTTDNYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 343 aa, synthetic construct
QVQLQESGGGSVQAGGSLSLSCAASGSIFGSKRMGWFRQAPGEQRDVVATIAVAGSTEYADSVKGRFTISRDNAKNTVYLQMNNLKPEDTAVYYCWMFTTDNYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 343 aa, synthetic construct
QVQLQESGGGSVQAGGSLSLSCAASGSIFGSKRMGWFRQAPGEQRDVVATITYRGTTEYADSVKGRFTISRDNAKNTVYLQMNNLKPEDTAVYYCWMYATANYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 343 aa, synthetic construct
QVQLQESGGGSVQAGGSLSLSCAASGSIFGFNAMGWFRQAPGEQRDVVATIAVAGSTEYADSVKGRFTISRDNAKNTVYLQMNNLKPEDTAVYYCWMFTTDNYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 343 aa, synthetic construct
QVQLQESGGGSVQAGGSLSLSCAASGSIFGFNAMGWFRQAPGEQRDVVATITYRGTTEYADSVKGRFTISRDNAKNTVYLQMNNLKPEDTAVYYCWMYATANYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 343 aa, synthetic construct
QVQLQESGGGSVQAGGSLSLSCAASGSIFGSKRMGWFRQAPGEQRDVVATIAVAGTTEYADSVKGRFTISRDNAKNTVYLQMNNLKPEDTAVYYCWMYATANYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 343 aa, synthetic construct
QVQLQESGGGSVQAGGSLSLSCAASGSIFGFNAMGWFRQAPGEQRDVVATIAVAGSTEYADSVKGRFTISRDNAKNTVYLQMNNLKPEDTAVYYCWMYATANYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 343 aa, synthetic construct
QVQLQESGGGLVQAGGSLSLSCAASGDINGSKRMGWFRQAPGKARDVVATITVRGTTEYEDSVKGRFTISRDNAKNTVYLQMNSLKPEDTAVYYCWMFTTTNYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 343 aa, synthetic construct
QVQLQESGGGSVQAGGSLRLSCAASEIIFGSKRMGWFRQAPGKQRDLVATITYHGTTKYADSVKGRFTISRDNANNTVYLQMNNLKPEDTAFYYCWMFTTDNYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 343 aa, synthetic construct
QVQLQESGGGLVQAGGSLSLSCAASGDIFGSKRMGWFRQAPGQQRDVVATITVGGTTEYADSVKGRFTISRDNAKNTVYLQMNSLKPEDTAVYYCWMFTTNNYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 177 aa, Homo sapiens
TGSSGSPNIPVQTISRAAAEKLFGNMEGDCPSDWKTDSTCRMVTSESKNVKLTVSNVGGGQVKDSAQNSVIIVDKNGRLVYLVENPGGYVASSKAATVTGKLVHANFGTKKDFEDLYTPVNGSIVIVRAGKITFAEKVANAESLNAIGVLIYMDQTKFPIVNAELSFGGAGGAGGAA

SEQ ID NO: 73 - skipped

SEQ ID NO: 74 - protein, 114 aa, synthetic construct
QVQLQESGGGLVQAGGSLRLSCAASGDINGSKRMGWFRQAPGKARDVVATITVRGTTEYEDSVKGRFTISRDNAKNTVYLQMNSLKPEDTAVYYCWMFTTTNYWGQGTQVTVSS

SEQ ID NO: 75 - protein, 114 aa, synthetic construct
QVQLQESGGGLVQAGGSLRLSCAASGDIFGSKRMGWFRQAPGQQRDVVATITVGGTTEYADSVKGRFTISRDNAKNTVYLQMNSLKPEDTAVYYCWMFTTNNYWGQGTQVTVSS

SEQ ID NO: 76 - protein, 113 aa, synthetic construct
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCGTDMEVWGKGTTVTVSS

SEQ ID NO: 77 - protein, 8 aa, synthetic construct
GGHHHHHH

SEQ ID NO: 78 - protein, 25 aa, synthetic construct
  SITE 1: note = Q or E
  SITE 5: note = Q or V
  SITE 11: note = S or L
  SITE 14: note = A or P
  SITE 19: note = S or R
XVQLXESGGGXVQXGGSLXLSCAAS

SEQ ID NO: 79 - protein, 17 aa, synthetic construct
  SITE 10: note = E or K
  SITE 11: note = Q or G
  SITE 12: note = R or L
  SITE 13: note = D or E
MGWFRQAPGXXXXVVAT

SEQ ID NO: 80 - protein, 38 aa, synthetic construct
  SITE 17: note = A or S
  SITE 21: note = V or I
  SITE 27: note = N or S
  SITE 29: note = K or R
EYADSVKGRFTISRDNXKNTXYLQMNXLXPEDTAVYYC

SEQ ID NO: 81 - protein, 11 aa, synthetic construct
  SITE 6: note = Q or L
WGQGTXVTVSS

SEQ ID NO: 82 - protein, 25 aa, synthetic construct
QVQLQESGGGSVQAGGSLRLSCAAS

SEQ ID NO: 83 - protein, 38 aa, synthetic construct
EYADSVKGRFTISRDNAKNTVYLQMNSLKPEDTAVYYC

SEQ ID NO: 84 - protein, 132 aa, synthetic construct
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSLE

SEQ ID NO: 85 - protein, 343 aa, synthetic construct
QVQLQESGGGLVQAGGSLRLSCAASGSIFGFNAMGWFRQAPGKERDLVATIAVAGSTEYADSVKGRFTISRDNAKNTMYLQMNSLKPEDTAVYYCWMYATANYWGQGTQVTVSSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK